SEQUENCE LISTING (62 sequences)

SEQ ID NO: 1 - DNA, 2205 bp, synthetic construct
  source 1..2205: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgcttcctggctacaagtacctcggacccttcaacggactcgacaagggggagcccgtcaacgaggcggacgcggcagccctcgaacacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaattgcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagccaatcaggagcttcaaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattccgcaatacggctacctgacgctcaacaatggcagccaagccgtgggacgttcatccttttactgcctggaatatttcccttctcagatgctgagaacgggcaacaactttcagttcagctacaccttcgaggacgtgcctttccacagcagctacgcgcacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaacagggacgtgtacctgcagggtcccatctgggccaagattcctcacacggatggcaactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaggtcagcgtggaaatcgagtgggagctgcagaaagaaaacagcaaacgctggaatccagagattcaatacacttccaactactacaaatctacaaatgtggactttgctgtcaacacggagggggtttatactgagcctcgccccattggcacccgttacctcacccgtcccctgtaa

SEQ ID NO: 2 - protein, 734 aa, synthetic construct
MSFVDHPPDWLEEVGEGLREFLGLEAGPPKPKPNQQHQDQARGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTIASGGGAPMADNNEGADGVGNASGNWHCDSTWLGDRVITTSTRTWALPTYNNHLYKQISSQSGASNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPAEFSATKFASFITQYSTGQVSVEIEWELQKENSKRWNPEIQYTSNYYKSTNVDFAVNTEGVYTEPRPIGTRYLTRPL

SEQ ID NO: 3 - DNA, 2211 bp, synthetic construct
  source 1..2211: mol_type = other DNA
atggctgccgatggttatcttccagattggctcgaggacaacctctctgagggcattcgcgagtggtgggacctgaaacctggagccccgaaacccaaagccaaccagcaaaagcaggacaaccgtcggggtcttgtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctgcaaagaagaggccggtagagccgtcacctcagcgttcccccgactcctccacgggcatcggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgacggagtgggtaatgcctcaggaaattggcattgcgattccacatggctgggcgacagagtcattaccaccagcacccgaacctgggccctgcccacctacaacaaccatctctacaagcaaatctccagccaatcaggagcttcaaacgacaaccactactttggctacagcaccccctgggggtattttgacttcaacagattccactgccacttctcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcgcctgaacggcagaaactcgttggttaatcccggcgtcgccatggcaactcacaaggacgatgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctccacagatcctgatcaagaacacgccggtacctgcggatcctccaacaacgttcagccaggcgaaattggcttccttcatcacgcagtacagcaccggacaagtcagcgtggaaatcgagtgggagctgcagaaggaaaacagcaagcgctggaacccggagattcagtacacttccaactacaacaagtctgttaatgtggactttactgtagacactaatggtgtttatagtgaaccccgccccattggcacccgttacctcacccgtaatctgtaa

SEQ ID NO: 4 - protein, 736 aa, synthetic construct
MAADGYLPDWLEDNLSEGIREWWDLKPGAPKPKANQQKQDNRRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPAKKRPVEPSPQRSPDSSTGIGKSGKQPARKRLNFGQTGDSESVPDPQPLGEPPAAPTSLGSNTMASGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYRLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYNKSVNVDFTVDTNGVYSEPRPIGTRYLTRNL

SEQ ID NO: 5 - DNA, 2205 bp, synthetic construct
  source 1..2205: mol_type = other DNA
atgactgacggttaccttccagattggctagaggacaacctctctgaaggcgttcgagagtggtgggcgctgcaacctggagcccctaaacccaaagccaaccagcaaaagcaggacggcggccggggtctggtgcttcctggctacaagtacctcggacccttcaacggactcgacaagggggagcccgtcaacgcggcggacgcagcggccctcgagcacgacaaagcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaacctctcggagaacctcccgcagccccctcaggtgtgggatctcttacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttctcaccacgtgactggcagcgactcatcaacaacaactggggattccggcccaagagactcagcttcaagctcttcaacatccaagttaaagaggtcacgcagaacgatggcacgacgactattgccaataaccttaccagcacggttcaagtgtttacggactcggagtatcagcttccgtacgtcctcggctctgcgcaccagggctgcctccctccgttcccggcggacgtgttcatgattccccagtacggctacctaacactcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcccctcatcgaccagtacctgtactacctggtcagaacgcaaacgactggaactggagggacgcagactctggcattcagccaagcgggtcctagctcaatggccaaccaggctagaaattgggtgcccggaccttgctaccggcagcagcgcgtctccacgacaaccaaccagaacaacaacagcaactttgcctggacgggagctgccaagtttaagctgaacggccgagactctctaatgaatccgggcgtggcaatggcttcccacaaggatgacgacgaccgcttcttcccttcgagcggggtcctgatttttggcaagcaaggagccgggaacgatggagtggactacagccaagtgctgattacagatgaggaagaaatcaaggctaccaaccccgtggccacagaagaatatggagcagtggccatcaacaaccaggccgccaatacgcaggcgcagaccggactcgtgcacaaccagggggtgattcccggcatggtgtggcagaatagagacgtgtacctgcagggtcccatctgggccaaaattcctcacacggacggcaactttcacccgtctcctctgatgggcggttttggactgaaacatccgcctccccagatatttatcaaaaacacccccgtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaagtgcagtatacatctaactatgcaaaatctgccaacgttgatttcactgtggacaacaatggactttatactgagcctcgccccattggcacccgataccttacccgacccctttaa

SEQ ID NO: 6 - protein, 734 aa, synthetic construct
MTDGYLPDWLEDNLSEGVREWWALQPGAPKPKANQQKQDGGRGLVLPGYKYLGPFNGLDKGEPVNAADAAALEHDKAYDQQLKAGDNPYLRYNHADAEFQERLQEDTSFGGNLGRAVFQAKKRLLEPLGLVEEAAKTAPGKKRPVEQSPQEPDSSAGIGKSGAQPAKKRLNFGQTGDTESVPDPQPLGEPPAAPSGVGSLTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQIFIKNTPVPANPSTTFSAAKFASFITQYSTGQVSVEIEWELQKENSKRWNPEVQYTSNYAKSANVDFTVDNNGLYTEPRPIGTRYLTRPL

SEQ ID NO: 7 - DNA, 2196 bp, synthetic construct
  source 1..2196: mol_type = other DNA
atggctgccgatggttatcttccagattggctagaggacaacctctctgagggcattcgcgagtggtgggacctgaaacctgcagaaggaaaacagcagaagcaggacgacggccggggtctggtgcttcctggctacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagtttcaggagcgtctgcaagaagatacgtcttttgggggcaacctcgggcgagcagtcttccaggccaagaagcgggttctcgaacctctcggcctggttgaggaacctgttaagacggctccgggaaaaaaagaggcctgtagagcagtctcctcagtaaccggactcctccgcgggtattggcaaatcgggtgcacagccgctaaaaagagactgaactttgggcagactggcgagtcagagtcagtccccgaccctcaaccaatcggagaaccaccagcaggcccctctggtctgggatctggtacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcagcttcaagctcttcaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgataaccaacgaagaagaaattaaaactactaacccggtagcaacggagtcctatggacaagtggccacaaaccaccagagtgcccaagcacaggcgcagaccggctgggttcaaaaccaaggaatacttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcggatcctccaacaacgttcagccaggcgaaattggcttccttcatcacgcagtacagcaccggacaggtcagcgtggaaatcgagtgggagctgcagaaggaaaacagcaagcgctggaacccggagatccagtacacttccaactattacaagtctaataatgttgaatttgctgttaatactgaaggtgtatatagtgaaccccgccccattggcacccgttacctcacccgtaatctgtaa

SEQ ID NO: 8 - protein, 731 aa, synthetic construct
MAADGYLPDWLEDNLSEGIREWWDLKPAEGKQQKQDDGRGLVLPGYKYLGPFNGLDKGEPVNEADAAALEHDKAYDRQLDSGDNPYLKYNHADAEFQERLQEDTSFGGNLGRAVFQAKKRVLEPLGLVEEPVKTAPGKKEACRAVSSVTGLLRGYWQIGCTAAKKRLNFGQTGESESVPDPQPIGEPPAGPSGLGSGTMAAGGGAPMADNNEGADGVGSSSGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITNEEEIKTTNPVATESYGQVATNHQSAQAQAQTGWVQNQGILPGMVWQDRDVYLQGPIWAKIPHTDGHFHPSPLMGGFGLKHPPPQILIKNTPVPADPPTTFSQAKLASFITQYSTGQVSVEIEWELQKENSKRWNPEIQYTSNYYKSNNVEFAVNTEGVYSEPRPIGTRYLTRNL

SEQ ID NO: 9 - DNA, 2211 bp, synthetic construct
  source 1..2211: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcggacgagcagtcttccaggcgaaaaagagggttcttgaacctctgggcctggttgaggaacctgttaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcagggtcccatctgggccaagattcctcacacggacggcaacttccacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggagaacagcaaacgctggaacccagagattcagtacacttcaaactactacaaatctacaaatgtggactttgctgttaatactgaaggtgtatatagtgaaccccgccccattggcaccagatacctcacccgtaatctgtaa

SEQ ID NO: 10 - protein, 736 aa, synthetic construct
MAADGYLPDWLEDNLSEGIREWWALKPGAPKPKANQQKQDDGRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEQSPQEPDSSAGIGKSGAQPAKKRLNFGQTGDTESVPDPQPIGEPPAAPSGVGTNTMATGSGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSTNVDFAVNTEGVYSEPRPIGTRYLTRNL

SEQ ID NO: 11 - DNA, 2211 bp, synthetic construct
  source 1..2211: mol_type = other DNA
atgactgacggttaccttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagccatcaccccagcgttctccagactcctctacgggcatcggcaagaaaggccagcagcccgctaaaaagagactcaattttggtcagactggcgactcagagtcagtccccgacccacaacctctcggagaacctccagcaacccccgctgctgtgggacctactacaatggcttcaggcggtggcgctccaatggcagacaataacgaaggcgccgacggagtgggtaatgcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagacatctgcggataacaacaacagtgaatactcgtggactggagctaccaagtaccacctcaatggcagagactctctggtgaatccgggcccggccatggcaagccacaaggacgatgaagaaaagttttttcctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggaatgaaacatccgcctcctcagatcctgattaagaatacacctgttcccgcggatcctccaactaccttcagtgcggcaaagtttgcttccttcatcacacagtactccacgggacaggtcagcgtggagatcgagtgggagctgcagaaggaaaacagcaaacgctggaacccagagattcagtacacttcaaactactacaaatctacaaatgtggactttactgtggacactaatggcgtgtattcagagcctcgccccattggcaccagatacctgactcgtaatctgtaa

SEQ ID NO: 12 - protein, 736 aa, synthetic construct
MTDGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPATPAAVGPTTMASGGGAPMADNNEGADGVGNASGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQGTTSGTTNQSRLLFSQAGPQSMSLQARNWLPGPCYRQQRLSKTSADNNNSEYSWTGATKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGMKHPPPQILIKNTPVPADPPTTFSAAKFASFITQYSTGQVSVEIEWELQKENSKRWNPEIQYTSNYYKSTNVDFTVDTNGVYSEPRPIGTRYLTRNL

SEQ ID NO: 13 - DNA, 2211 bp, synthetic construct
  source 1..2211: mol_type = other DNA
atggctgccgatggttatcttccagattggctcgaggacaacctctctgagggcattcgcgagtggtgggacctgaaacctggagccccgaaacccaaagccaaccagcaaaagcaggacaaccgtcggggtcttgtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctgcaaagaagaggccggtagagccgtcacctcagcgttcccccgactcctccacgggcatcggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgacggagtgggtaatgcctcaggaaattggcattgcgattccacatggctgggcgacagagtcattaccaccagcacccgaacctgggccctgcccacctacaacaaccatctctacaagcaaatctccagccaatcaggagcttcaaacgacaaccactactttggctacagcaccccctgggggtattttgacttcaacagattccactgccacttctcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcgcctgaacggcagaaactcgttggttaatcccggcgtcgccatggcaactcacaaggacgatgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaacagggacgtgtacctgcagggtcccatctgggccaagattcctcacacggatggcaactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaggtcagcgtggaaatcgagtgggagctgcagaaagaaaacagcaaacgctggaatccagagattcaatacacttccaactactacaaatctacaaatgtggactttgctgtcaacacggagggggtttatactgagcctcgccccattggcacccgttacctcacccgtcccctgtaa

SEQ ID NO: 14 - protein, 736 aa, synthetic construct
MAADGYLPDWLEDNLSEGIREWWDLKPGAPKPKANQQKQDNRRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPAKKRPVEPSPQRSPDSSTGIGKSGKQPARKRLNFGQTGDSESVPDPQPLGEPPAAPTSLGSNTMASGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYRLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPAEFSATKFASFITQYSTGQVSVEIEWELQKENSKRWNPEIQYTSNYYKSTNVDFAVNTEGVYTEPRPIGTRYLTRPL

SEQ ID NO: 15 - DNA, 2205 bp, synthetic construct
  source 1..2205: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgcttcctggctacaagtacctcggacccttcaacggactcgacaagggggagcccgtcaacgaggcggacgcggcagccctcgaacacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaattgcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagccaatcaggagcttcaaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattccgcaatacggctacctgacgctcaacaatggcagccaagccgtgggacgttcatccttttactgcctggaatatttcccttctcagatgctgagaacgggcaacaactttcagttcagctacaccttcgaggacgtgcctttccacagcagctacgcgcacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctccacagatcctgatcaagaacacgccggtacctgcggatcctccaacaacgttcagccaggcgaaattggcttccttcatcacgcagtacagcaccggacaagtcagcgtggaaatcgagtgggagctgcagaaggaaaacagcaagcgctggaacccggagattcagtacacttccaactacaacaagtctgttaatgtggactttactgtagacactaatggtgtttatagtgaaccccgccccattggcacccgttacctcacccgtaatctgtaa

SEQ ID NO: 16 - protein, 734 aa, synthetic construct
MSFVDHPPDWLEEVGEGLREFLGLEAGPPKPKPNQQHQDQARGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTIASGGGAPMADNNEGADGVGNASGNWHCDSTWLGDRVITTSTRTWALPTYNNHLYKQISSQSGASNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYNKSVNVDFTVDTNGVYSEPRPIGTRYLTRNL

SEQ ID NO: 17 - DNA, 2208 bp, synthetic construct
  source 1..2208: mol_type = other DNA
atggctgccgatggttatcttccagattggctcgaggacaacctctctgagggcattcgcgagtggtgggacctgaaacctggagccccgaaacccaaagccaaccagcaaaagcaggacaaccgtcggggtcttgtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaattgcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagccaatcaggagcttcaaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattccgcaatacggctacctgacgctcaacaatggcagccaagccgtgggacgttcatccttttactgcctggaatatttcccttctcagatgctgagaacgggcaacaactttcagttcagctacaccttcgaggacgtgcctttccacagcagctacgcgcacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaacagggacgtgtacctgcagggtcccatctgggccaagattcctcacacggatggcaactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaggtcagcgtggaaatcgagtgggagctgcagaaagaaaacagcaaacgctggaatccagagattcaatacacttccaactactacaaatctacaaatgtggactttgctgtcaacacggagggggtttatactgagcctcgccccattggcacccgttacctcacccgtcccctgtaa

SEQ ID NO: 18 - protein, 735 aa, synthetic construct
MAADGYLPDWLEDNLSEGIREWWDLKPGAPKPKANQQKQDNRRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTIASGGGAPMADNNEGADGVGNASGNWHCDSTWLGDRVITTSTRTWALPTYNNHLYKQISSQSGASNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPAEFSATKFASFITQYSTGQVSVEIEWELQKENSKRWNPEIQYTSNYYKSTNVDFAVNTEGVYTEPRPIGTRYLTRPL

SEQ ID NO: 19 - DNA, 2211 bp, synthetic construct
  source 1..2211: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgcttcctggctacaagtacctcggacccttcaacggactcgacaagggggagcccgtcaacgaggcggacgcggcagccctcgaacacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcagtcgccacaagagccagactcctcctcgggcattggcaagacaggccagcagcccgctaaaaggagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctacaacaaccacctctacaagcaaatctccaacgggacatcgggaggaagcaccaacgacaacacctattttggctacagcaccccctgggggtattttgatttcaacagattccactgccacttttcaccacgtgactggcagcgactcatcaacaacaactggggattccggcccaagagactcagcttcaagctcttcaacatccaggtcaaggaggtcacgcagaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttcagttcagctacaccttcgaggacgtgcctttccacagcagctacgcgcacagccagagcctggaccggctgatgaatcctctcatcgaccagtacctgtattacctgaacagaactcagtccacgggaggtaccgcaggaactcagcagttgctattttctcaggccgggcctaataacatgtcggctcaggccaagaattggttacctggaccttgcttccggcaacaaagagtctccaaaacgctggatcaaaacaacaatagcaactttgcctggactgctgggaccaagtatcatctgaatggcagagactctctggtaaatcccggtgtcgctatggcaacccacaaggacgacgaagagcgattttttccgtccagcggagtcttaatgtttgggaaacagggagctggaaaggacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagcgagcctcgccccattggcacccgttacctcacccgcaacctgtaa

SEQ ID NO: 20 - protein, 736 aa, synthetic construct
MSFVDHPPDWLEEVGEGLREFLGLEAGPPKPKPNQQHQDQARGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKRRLNFGQTGDSESVPDPQPLGEPPATPAAVGPTTMASGGGAPMADNNEGADGVGNASGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNDGVTTIANNLTSTVQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLNRTQSTGGTAGTQQLLFSQAGPNNMSAQAKNWLPGPCFRQQRVSKTLDQNNNSNFAWTAGTKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 21 - DNA, 2211 bp, synthetic construct
  source 1..2211: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacgggctcgacaagggggagcccgtcaacgcggcggacgcagcggccctcgagcacgacaaggcctacgaccagcagctcgacagcggagacaacccgtacctcaagtacaaccacgccgacgcggagtttcaggagcgccttaaagaagatacgtcttttgggggcaacctcgggcgagcagtcttccaggccaagaagcgggttctcgaacctctcggtctggttgaggaaggcgctaagacggctcctgcaaagaagaggccggtagagccgtcacctcagcgttcccccgactcctccacgggcatcggcaagaaaggccagcagcccgccagaaagagactcaatttcggtcagactggcgactcagagtcagtccccgaccctcaaccaatcggagaaccccccgcaggcccctctggtctgggatctggtacaatggctgcaggcggtggcgctccaatggcagacaataacgaaggcgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttttcaccacgtgactggcaaagactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaaaaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccgaccaccttcaaccagtcaaagctgaactctttcatcacgcaatacagcaccggacaggtcagcgtggaaatcgagtgggagctgcagaaggagaacagcaaacgctggaacccagagattcagtacacttcaaactactacaaatctacaaatgtggactttgctgtggacactaatggcgtgtattcagagcctcgccccattggcaccagatacctgactcgtaatctgtaa

SEQ ID NO: 22 - protein, 736 aa, synthetic construct
MSFVDHPPDWLEEVGEGLREFLGLEAGPPKPKPNQQHQDQARGLVLPGYNYLGPGNGLDKGEPVNAADAAALEHDKAYDQQLDSGDNPYLKYNHADAEFQERLKEDTSFGGNLGRAVFQAKKRVLEPLGLVEEGAKTAPAKKRPVEPSPQRSPDSSTGIGKKGQQPARKRLNFGQTGDSESVPDPQPIGEPPAGPSGLGSGTMAAGGGAPMADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKKGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTTFNQSKLNSFITQYSTGQVSVEIEWELQKENSKRWNPEIQYTSNYYKSTNVDFAVDTNGVYSEPRPIGTRYLTRNL

SEQ ID NO: 23 - DNA, 2211 bp, synthetic construct
  source 1..2211: mol_type = other DNA
atggctgccgatggttatcttccagattggctcgaggacaacctctctgagggcattcgcgagtggtgggacctgaaacctggagccccgaaacccaaagccaaccagcaaaagcaggacaaccgtcggggtcttgtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctgcaaagaagaggccggtagagccgtcacctcagcgttcccccgactcctccacgggcatcggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgacggagtgggtaatgcctcaggaaattggcattgcgattccacatggctgggcgacagagtcatcaccaccagcacccgaacatgggccttgcccacctataacaaccacctctacaagcaaatctccagccaatcaggagcttcaaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattccgcaatacggctacctgacgctcaacaatggcagccaagccgtgggacgttcatccttttactgcctggaatatttcccttctcagatgctgagaacgggcaacaactttcagttcagctacaccttcgaggacgtgcctttccacagcagctacgcgcacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaacagggacgtgtacctgcagggtcccatctgggccaagattcctcacacggatggcaactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaggtcagcgtggaaatcgagtgggagctgcagaaagaaaacagcaaacgctggaatccagagattcaatacacttccaactactacaaatctacaaatgtggactttgctgtcaacacggagggggtttatactgagcctcgccccattggcacccgttacctcacccgtcccctgtaa

SEQ ID NO: 24 - protein, 736 aa, synthetic construct
MAADGYLPDWLEDNLSEGIREWWDLKPGAPKPKANQQKQDNRRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPAKKRPVEPSPQRSPDSSTGIGKSGKQPARKRLNFGQTGDSESVPDPQPLGEPPAAPTSLGSNTMASGGGAPMADNNEGADGVGNASGNWHCDSTWLGDRVITTSTRTWALPTYNNHLYKQISSQSGASNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPAEFSATKFASFITQYSTGQVSVEIEWELQKENSKRWNPEIQYTSNYYKSTNVDFAVNTEGVYTEPRPIGTRYLTRPL

SEQ ID NO: 25 - DNA, 2208 bp, synthetic construct
  source 1..2208: mol_type = other DNA
atgactgacggttaccttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggagagccggtcaacgcagcagacgccgcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaaccacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtcctcggctcggcgcatcaaggatgcctcccgccgttcccagcagacgtcttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggacattttcacccctctcccctcatgggtggattcggacttaaacaccctcctccacagattctcatcaagaacaccccggtacctgcggatcctccgaccaccttcaaccagtcaaagctgaactctttcatcacgcaatacagcaccggacaggtcagcgtggaaattgaatgggagctgcagaaggaaaacagcaaacgctggaacccagagattcagtacacttcaaactactacaaatctacaaatgtggactttgctgtcaatacagagggaacttattctgagcctcgccccattggcaccagatacctcacccgtaatctgtaa

SEQ ID NO: 26 - protein, 735 aa, synthetic construct
MTDGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTNVDFAVNTEGTYSEPRPIGTRYLTRNL

SEQ ID NO: 27 - DNA, 2205 bp, synthetic construct
  source 1..2205: mol_type = other DNA
atgactgacggttaccttccagattggctagaggacaacctctctgaaggcgttcgagagtggtgggcgctgcaacctggagcccctaaacccaaagccaaccagcaaaagcaggacggcggccggggtctggtgcttcctggctacaagtacctcggacccttcaacggactcgacaagggggagcccgtcaacgcggcggacgcagcggccctcgagcacgacaaagcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaattgcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagccaatcaggagcttcaaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattccgcaatacggctacctgacgctcaacaatggcagccaagccgtgggacgttcatccttttactgcctggaatatttcccttctcagatgctgagaacgggcaacaactttcagttcagctacaccttcgaggacgtgcctttccacagcagctacgcgcacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaacagggacgtgtacctgcagggtcccatctgggccaagattcctcacacggatggcaactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaggtcagcgtggaaatcgagtgggagctgcagaaagaaaacagcaaacgctggaatccagagattcaatacacttccaactactacaaatctacaaatgtggactttgctgtcaacacggagggggtttatactgagcctcgccccattggcacccgttacctcacccgtcccctgtaa

SEQ ID NO: 28 - protein, 734 aa, synthetic construct
MTDGYLPDWLEDNLSEGVREWWALQPGAPKPKANQQKQDGGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTIASGGGAPMADNNEGADGVGNASGNWHCDSTWLGDRVITTSTRTWALPTYNNHLYKQISSQSGASNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPAEFSATKFASFITQYSTGQVSVEIEWELQKENSKRWNPEIQYTSNYYKSTNVDFAVNTEGVYTEPRPIGTRYLTRPL

SEQ ID NO: 29 - DNA, 2205 bp, synthetic construct
  source 1..2205: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgcttcctggctacaagtacctcggacccttcaacggactcgacaagggggagcccgtcaacgaggcggacgcggcagccctcgaacacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaattgcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagccaatcaggagcttcaaacgacaacacctacttcggctacagcaccccctgggggtattttgactttaacagattccactgccacttctcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcgcctgaacggcagaaactcgttggttaatcccggcgtcgccatggcaactcacaaggacgatgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctccacagatcctgatcaagaacacgccggtacctgcggatcctccaacaacgttcagccaggcgaaattggcttccttcatcacgcagtacagcaccggacaagtcagcgtggaaatcgagtgggagctgcagaaggaaaacagcaagcgctggaacccggagattcagtacacttccaactacaacaagtctgttaatgtggactttactgtagacactaatggtgtttatagtgaaccccgccccattggcacccgttacctcacccgtaatctgtaa

SEQ ID NO: 30 - protein, 734 aa, synthetic construct
MSFVDHPPDWLEEVGEGLREFLGLEAGPPKPKPNQQHQDQARGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTIASGGGAPMADNNEGADGVGNASGNWHCDSTWLGDRVITTSTRTWALPTYNNHLYKQISSQSGASNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYRLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYNKSVNVDFTVDTNGVYSEPRPIGTRYLTRNL

SEQ ID NO: 31 - DNA, 2208 bp, synthetic construct
  source 1..2208: mol_type = other DNA
atgactgacggttaccttccagattggctagaggacaacctctctgaaggcgttcgagagtggtgggcgctgcaacctggagcccctaaacccaaagccaaccagcaaaagcaggacggcggccggggtctggtgcttcctggctacaagtacctcggacccttcaacggactcgacaagggggagcccgtcaacgcggcggacgcagcggccctcgagcacgacaaagcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaacctctcggagaacctcccgcagccccctcaggtgtgggatctcttacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttctcaccacgtgactggcagcgactcatcaacaacaactggggattccggcccaagagactcagcttcaagctcttcaacatccaagttaaagaggtcacgcagaacgatggcacgacgactattgccaataaccttaccagcacggttcaagtgtttacggactcggagtatcagcttccgtacgtcctcggctctgcgcaccagggctgcctgcctccgttcccggcggacgtcttcatgattccgcaatacggctacctgacgctcaacaatggcagccaagccgtgggacgttcatccttttactgcctggaatatttcccttctcagatgctgagaacgggcaacaactttcagttcagctacaccttcgaggacgtgcctttccacagcagctacgcgcacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaacagggacgtgtacctgcagggtcccatctgggccaagattcctcacacggatggcaactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaggtcagcgtggaaatcgagtgggagctgcagaaagaaaacagcaaacgctggaatccagagattcaatacacttccaactactacaaatctacaaatgtggactttgctgtcaacacggagggggtttatactgagcctcgccccattggcacccgttacctcacccgtcccctgtaa

SEQ ID NO: 32 - protein, 735 aa, synthetic construct
MTDGYLPDWLEDNLSEGVREWWALQPGAPKPKANQQKQDGGRGLVLPGYKYLGPFNGLDKGEPVNAADAAALEHDKAYDQQLKAGDNPYLRYNHADAEFQERLQEDTSFGGNLGRAVFQAKKRLLEPLGLVEEAAKTAPGKKRPVEQSPQEPDSSAGIGKSGAQPAKKRLNFGQTGDTESVPDPQPLGEPPAAPSGVGSLTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPAEFSATKFASFITQYSTGQVSVEIEWELQKENSKRWNPEIQYTSNYYKSTNVDFAVNTEGVYTEPRPIGTRYLTRPL

SEQ ID NO: 33 - DNA, 2208 bp, synthetic construct
  source 1..2208: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgcttcctggctacaagtacctcggacccttcaacggactcgacaagggggagcccgtcaacgaggcggacgcggcagccctcgaacacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcagtcgccacaagagccagactcctcctcgggcatcggcaagacaggccagcagcccgctaaaaagagactcaattttggtcagactggcgactcagagtcagtccccgaccctcaacctctcggagaacctccagcaacccccgctgctgtgggacctactacaatggcttcaggcggtggcgcaccaatggcagacaataacgagggtgccgacggagtgggtaatgcctcaggaaattggcattgcgattccacatggctgggcgacagagtcatcaccaccagcacccgaacctgggccctccccacctacaacaaccacctctacaagcaaatctccagtgcttcaacgggggccagcaacgacaaccactacttcggctacagcaccccctgggggtattttgatttcaacagattccactgccacttttcaccacgtgactggcaaagactcatcaacaacaactggggattccgacccaagagactcaacttcaagctctttaacattcaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcccctcatcgaccagtacttgtactacctgaacagaactcagaatcagtccggaagtgcccaaaacaaggacttgctgtttagccgtggatctccagctggcatgtctgttcagcccaaaaactggctacctggaccctgttaccggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggactgtggcagtcaatctccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaaggacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaaacgctggaatcccgaaattcagtacacctccaactttgaaaagcagactggtgtggactttgccgtagacactaatggtgtttatagtgaacctcgccctattggcactcgttatctgacacgtaatctgtaa

SEQ ID NO: 34 - protein, 735 aa, synthetic construct
MSFVDHPPDWLEEVGEGLREFLGLEAGPPKPKPNQQHQDQARGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNLQSSSTDPATGDVHAMGALPGMVWQDRDVYLQGPIWAKIPHTDGHFHPSPLMGGFGLKDPPPQILIKNTPVPANPPAEFSATKFASFITQYSTGQVSVEIEWELQKENSKRWNPEIQYTSNFEKQTGVDFAVDTNGVYSEPRPIGTRYLTRNL

SEQ ID NO: 35 - DNA, 2208 bp, synthetic construct
  source 1..2208: mol_type = other DNA
atggctgccgatggttatcttccagattggctcgaggacaacctctctgagggcattcgcgagtggtgggacctgaaacctggagccccgaaacccaaagccaaccagcaaaagcaggacaaccgtcggggtcttgtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaacctctcggagaacctcccgcagccccctcaggtgtgggatctcttacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttctcaccacgtgactggcagcgactcatcaacaacaactggggattccggcccaagagactcagcttcaagctcttcaacatccaagttaaagaggtcacgcagaacgatggcacgacgactattgccaataaccttaccagcacggttcaagtgtttacggactcggagtatcagcttccgtacgtcctcggctctgcgcaccagggctgcctccctccgttcccggcggacgtgttcatgattccccagtacggctacctaacactcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcccctcatcgaccagtacctgtactacctggtcagaacgcaaacgactggaactggagggacgcagactctggcattcagccaagcgggtcctagctcaatggccaaccaggctagaaattgggtgcccggaccttgctaccggcagcagcgcgtctccacgacaaccaaccagaacaacaacagcaactttgcctggacgggagctgccaagtttaagctgaacggccgagactctctaatgaatccgggcgtggcaatggcttcccacaaggatgacgacgaccgcttcttcccttcgagcggggtcctgatttttggcaagcaaggagccgggaacgatggagtggactacagccaagtgctgattacagatgaggaagaaatcaaggctaccaaccccgtggccacagaagaatatggagcagtggccatcaacaaccaggccgccaatacgcaggcgcagaccggactcgtgcacaaccagggggtgattcccggcatggtgtggcagaatagagacgtgtacctgcagggtcccatctgggccaaaattcctcacacggacggcaactttcacccgtctcctctgatgggcggttttggactgaaacatccgcctccccagatatttatcaaaaacacccccgtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaagtgcagtatacatctaactatgcaaaatctgccaacgttgatttcactgtggacaacaatggactttatactgagcctcgccccattggcacccgataccttacccgacccctttaa

SEQ ID NO: 36 - protein, 735 aa, synthetic construct
MAADGYLPDWLEDNLSEGIREWWDLKPGAPKPKANQQKQDNRRGLVLPGYKYLGPFNGLDKGEPVNAADAAALEHDKAYDQQLQAGDNPYLRYNHADAEFQERLQEDTSFGGNLGRAVFQAKKRLLEPLGLVEEAAKTAPGKKRPVEQSPQEPDSSAGIGKSGAQPAKKRLNFGQTGDTESVPDPQPLGEPPAAPSGVGSLTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQIFIKNTPVPANPSTTFSAAKFASFITQYSTGQVSVEIEWELQKENSKRWNPEVQYTSNYAKSANVDFTVDNNGLYTEPRPIGTRYLTRPL

SEQ ID NO: 37 - DNA, 2196 bp, synthetic construct
  source 1..2196: mol_type = other DNA
atggctgccgatggttatcttccagattggctagaggacaacctctctgagggcattcgcgagtggtgggacctgaaacctgcagaaggaaaacagcagaagcaggacgacggccggggtctggtgcttcctggctacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaattgcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagccaatcaggagcttcaaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattccgcaatacggctacctgacgctcaacaatggcagccaagccgtgggacgttcatccttttactgcctggaatatttcccttctcagatgctgagaacgggcaacaactttcagttcagctacaccttcgaggacgtgcctttccacagcagctacgcgcacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaacagggacgtgtacctgcagggtcccatctgggccaagattcctcacacggatggcaactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaggtcagcgtggaaatcgagtgggagctgcagaaagaaaacagcaaacgctggaatccagagattcaatacacttccaactactacaaatctacaaatgtggactttgctgtcaacacggagggggtttatactgagcctcgccccattggcacccgttacctcacccgtcccctgtaa

SEQ ID NO: 38 - protein, 731 aa, synthetic construct
MAADGYLPDWLEDNLSEGIREWWDLKPAEGKQQKQDDGRGLVLPGYKYLGPFNGLDKGEPVNEADAAALEHDKAYDRQLDSGDNPYLKYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTIASGGGAPMADNNEGADGVGNASGNWHCDSTWLGDRVITTSTRTWALPTYNNHLYKQISSQSGASNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPAEFSATKFASFITQYSTGQVSVEIEWELQKENSKRWNPEIQYTSNYYKSTNVDFAVNTEGVYTEPRPIGTRYLTRPL

SEQ ID NO: 39 - DNA, 2208 bp, synthetic construct
  source 1..2208: mol_type = other DNA
atgactgacggttaccttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagccatcaccccagcgttctccagactcctctacgggcatcggcaagaaaggccagcagcccgctaaaaagagactcaattttggtcagactggcgactcagagtcagtccccgacccacaacctctcggagaacctccagcaacccccgctgctgtgggacctactacaatggcttcaggcggtggcgctccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgacttcaacagattccactgccacttctcaccacgtgactggcagcgactcatcaacaacaactggggattccggcccaagagactcagcttcaagctcttcaacatccaagttaaagaggtcacgcagaacgatggcacgacgactattgccaataaccttaccagcacggttcaagtgtttacggactcggagtatcagcttccgtacgtcctcggctctgcgcaccagggctgcctccctccgttcccggcggacgtgttcatgattccccagtacggctacctaacactcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcccctcatcgaccagtacctgtactacctggtcagaacgcaaacgactggaactggagggacgcagactctggcattcagccaagcgggtcctagctcaatggccaaccaggctagaaattgggtgcccggaccttgctaccggcagcagcgcgtctccacgacaaccaaccagaacaacaacagcaactttgcctggacgggagctgccaagtttaagctgaacggccgagactctctaatgaatccgggcgtggcaatggcttcccacaaggatgacgacgaccgcttcttcccttcgagcggggtcctgatttttggcaagcaaggagccgggaacgatggagtggactacagccaagtgctgattacagatgaggaagaaatcaaggctaccaaccccgtggccacagaagaatatggagcagtggccatcaacaaccaggccgccaatacgcaggcgcagaccggactcgtgcacaaccagggggtgattcccggcatggtgtggcagaatagagacgtgtacctgcagggtcccatctgggccaaaattcctcacacggacggcaactttcacccgtctcctctgatgggcggttttggactgaaacatccgcctccccagatatttatcaaaaacacccccgtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaagtgcagtatacatctaactatgcaaaatctgccaacgttgatttcactgtggacaacaatggactttatactgagcctcgccccattggcacccgataccttacccgacccctttaa

SEQ ID NO: 40 - protein, 735 aa, synthetic construct
MTDGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQIFIKNTPVPANPSTTFSAAKFASFITQYSTGQVSVEIEWELQKENSKRWNPEVQYTSNYAKSANVDFTVDNNGLYTEPRPIGTRYLTRPL

SEQ ID NO: 41 - DNA, 2211 bp, synthetic construct
  source 1..2211: mol_type = other DNA
atggctgccgatggttatcttccagattggctcgaggacaacctctctgagggcattcgcgagtggtgggacctgaaacctggagccccgaaacccaaagccaaccagcaaaagcaggacaaccgtcggggtcttgtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctgcaaagaagaggccggtagagccgtcacctcagcgttcccccgactcctccacgggcatcggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgacggagtgggtaatgcctcaggaaattggcattgcgattccacatggctgggcgacagagtcattaccaccagcacccgaacctgggccctgcccacctacaacaaccatctctacaagcaaatctccagccaatcaggagcttcaaacgacaaccactactttggctacagcaccccctgggggtattttgacttcaacagattccactgccacttctcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcgcctgaacggcagaaactcgttggttaatcccggcgtcgccatggcaactcacaaggacgatgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctccacagatcctgatcaagaacacgccggtacctgcggatcctccaacaacgttcagccaggcgaaattggcttccttcatcacgcagtacagcaccggacaagtcagcgtggaaatcgagtgggagctgcagaaggaaaacagcaagcgctggaacccggagattcagtacacttccaactactacaaatctacaaatgtggactttgctgtcaacacggagggggtttatactgagcctcgccccattggcacccgttacctcacccgtcccctgtaa

SEQ ID NO: 42 - protein, 736 aa, synthetic construct
MAADGYLPDWLEDNLSEGIREWWDLKPGAPKPKANQQKQDNRRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPAKKRPVEPSPQRSPDSSTGIGKSGKQPARKRLNFGQTGDSESVPDPQPLGEPPAAPTSLGSNTMASGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYRLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGVYTEPRPIGTRYLTRPL

SEQ ID NO: 43 - DNA, 2211 bp, synthetic construct
  source 1..2211: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacgggctcgatcgaggagagccggtcaacgaggcagacgccgcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaacctcttggtctggttgaggaaggcgctaagacggctcctggaaagaaacgtccggtagagcagtcgccacaagagccagactcctcctcgggcatcggcaagacaggccagcagcccgctaaaaagagactcaatttcggtcagactggcgactcagagtcagtccccgatccacaacctctcggagaacctccagcagccccctctggtctgggaactaatacgatggctacaggcagtggcgcaccaatggcagacaataacgagggcgccgacggagtgggtaattcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacggttcaagtgtttacggactcggagtatcagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacatccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaa

SEQ ID NO: 44 - protein, 736 aa, synthetic construct
MSFVDHPPDWLEEVGEGLREFLGLEAGPPKPKPNQQHQDQARGLVLPGYNYLGPGNGLDRGEPVNEADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGTNTMATGSGAPMADNNEGADGVGNSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTQNEGTKTIANNLTSTVQVFTDSEYQLPYVLGSAHQGCLPPFPADVFMIPQYGYLTLNNGSQAVGRSSFYCLEYFPSQMLRTGNNFEFSYQFEDVPFHSSYAHIQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKFASFITQYSTGQVSVEIEWELQKENSKRWNPEIQYTSNFEKQTGVDFAVDSQGVYSEPRPIGTRYLTRNL

SEQ ID NO: 45 - DNA, 2211 bp, synthetic construct
  source 1..2211: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgcttcctggctacaagtacctcggacccttcaacggactcgacaagggggagcccgtcaacgaggcggacgcggcagccctcgaacacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagccatcaccccagcgttctccagactcctctacgggcatcggcaagaaaggccagcagcccgctaaaaagagactcaattttggtcagactggcgactcagagtcagtccccgacccacaacctctcggagaacctccagcaacccccgctgctgtgggacctactacaatggcttcaggcggtggcgctccaatggcagacaataacgaaggcgccgacggagtgggtaatgcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagacatctgcggataacaacaacagtgaatactcgtggactggagctaccaagtaccacctcaatggcagagactctctggtgaatccgggcccggccatggcaagccacaaggacgatgaagaaaagttttttcctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggaatgaaacatccgcctcctcagatcctgattaagaatacacctgttcccgcggatcctccaactaccttcagtgcggcaaagtttgcttccttcatcacacagtactccacgggacaggtcagcgtggagatcgagtgggagctgcagaaggaaaacagcaaacgctggaacccagagattcagtacacttcaaactactacaaatctacaaatgtggactttactgtggacactaatggcgtgtattcagagcctcgccccattggcaccagatacctgactcgtaatctgtaa

SEQ ID NO: 46 - protein, 736 aa, synthetic construct
MSFVDHPPDWLEEVGEGLREFLGLEAGPPKPKPNQQHQDQARGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPLGEPPATPAAVGPTTMASGGGAPMADNNEGADGVGNASGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQGTTSGTTNQSRLLFSQAGPQSMSLQARNWLPGPCYRQQRLSKTSADNNNSEYSWTGATKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGMKHPPPQILIKNTPVPADPPTTFSAAKFASFITQYSTGQVSVEIEWELQKENSKRWNPEIQYTSNYYKSTNVDFTVDTNGVYSEPRPIGTRYLTRNL

SEQ ID NO: 47 - DNA, 2205 bp, synthetic construct
  source 1..2205: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgcttcctggctacaagtacctcggacccttcaacggactcgacaagggggagcccgtcaacgaggcggacgcggcagccctcgaacacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaattgcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagccaatcaggagcttcaaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattccgcaatacggctacctgacgctcaacaatggcagccaagccgtgggacgttcatccttttactgcctggaatatttcccttctcagatgctgagaacgggcaacaactttcagttcagctacaccttcgaggacgtgcctttccacagcagctacgcgcacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaacagggacgtgtacctgcagggtcccatctgggccaagattcctcacacggatggcaactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaggtcagcgtggaaatcgagtgggagctgcagaaagaaaacagcaaacgctggaatccagagattcaatacacttccaactactacaaatctacaaatgtggactttgctgtcaacacggagggggtttatactgagcctcgccccattggcacccgttacctcacccgtaatctgtaa

SEQ ID NO: 48 - protein, 734 aa, synthetic construct
MSFVDHPPDWLEEVGEGLREFLGLEAGPPKPKPNQQHQDQARGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTIASGGGAPMADNNEGADGVGNASGNWHCDSTWLGDRVITTSTRTWALPTYNNHLYKQISSQSGASNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPAEFSATKFASFITQYSTGQVSVEIEWELQKENSKRWNPEIQYTSNYYKSTNVDFAVNTEGVYTEPRPIGTRYLTRNL

SEQ ID NO: 49 - DNA, 2205 bp, synthetic construct
  source 1..2205: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgcttcctggctacaagtacctcggacccttcaacggactcgacaagggggagcccgtcaacgaggcggacgcggcagccctcgaacacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaattgcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagccaatcaggagcttcaaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgataaccaacgaagaagaaattaaaactactaacccggtagcaacggagtcctatggacaagtggccacaaaccaccagagtgcccaagcacaggcgcagaccggctgggttcaaaaccaaggaatacttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcggatcctccaacaacgttcagccaggcgaaattggcttccttcatcacgcagtacagcaccggacaggtcagcgtggaaatcgagtgggagctgcagaaggaaaacagcaagcgctggaacccggagatccagtacacttccaactattacaagtctaataatgttgaatttgctgttaatactgaaggtgtatatagtgaaccccgccccattggcacccgttacctcacccgtaatctgtaa

SEQ ID NO: 50 - protein, 734 aa, synthetic construct
MSFVDHPPDWLEEVGEGLREFLGLEAGPPKPKPNQQHQDQARGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTIASGGGAPMADNNEGADGVGNASGNWHCDSTWLGDRVITTSTRTWALPTYNNHLYKQISSQSGASNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITNEEEIKTTNPVATESYGQVATNHQSAQAQAQTGWVQNQGILPGMVWQDRDVYLQGPIWAKIPHTDGHFHPSPLMGGFGLKHPPPQILIKNTPVPADPPTTFSQAKLASFITQYSTGQVSVEIEWELQKENSKRWNPEIQYTSNYYKSNNVEFAVNTEGVYSEPRPIGTRYLTRNL

SEQ ID NO: 51 - DNA, 2205 bp, synthetic construct
  source 1..2205: mol_type = other DNA
atgtcttttgttgatcaccctccagattggttggaagaagttggtgaaggtcttcgcgagtttttgggccttgaagcgggcccaccgaaaccaaaacccaatcagcaaaagcaggacaacggccggggtctggtgcttcctggctacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggccaaaaagaggcttctcgaacctctcggtctggttgaggaaggcgctaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagcctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaacaaccacctctacaaacaaatttccagccaatcaggagcctcgaacgacaatcactactttggctacagcaccccttgggggtattttgacttcaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattccgcaatacggctacctgacgctcaacaatggcagccaagccgtgggacgttcatccttttactgcctggaatatttcccttctcagatgctgagaacgggcaacaactttcagttcagctacaccttcgaggacgtgcctttccacagcagctacgcgcacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaacagggacgtgtacctgcagggtcccatctgggccaagattcctcacacggatggcaactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaggtcagcgtggaaatcgagtgggagctgcagaaagaaaacagcaaacgctggaatccagagattcaatacacttccaactactacaaatctacaaatgtggactttgctgtcaacacggagggggtttatactgagcctcgccccattggcacccgttacctcacccgtcccctgtaa

SEQ ID NO: 52 - protein, 734 aa, synthetic construct
MSFVDHPPDWLEEVGEGLREFLGLEAGPPKPKPNQQKQDNGRGLVLPGYKYLGPFNGLDKGEPVNEADAAALEHDKAYDRQLDSGDNPYLKYNHADAEFQERLKEDTSFGGNLGRAVFQAKKRLLEPLGLVEEGAKTAPGKKRPVEHSPVEPDSSSGTGKAGQQPARKRLNFGQTGDADSVPDPQPLGQPPATPAAVGPTTMASGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPAEFSATKFASFITQYSTGQVSVEIEWELQKENSKRWNPEIQYTSNYYKSTNVDFAVNTEGVYTEPRPIGTRYLTRPL

SEQ ID NO: 53 - DNA, 2208 bp, synthetic construct
  source 1..2208: mol_type = other DNA
atgtcttttgttgatcaccctccagattggctcgaggacaacctctctgaaggcgttcgagagtggtgggacctgaaacctggagcccccaagcccaaggccaaccagcagaagcaggacaacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcatttgggggcaacctcgggcgagcagtcttccaggccaagaagagggttctcgaacctcttggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaattgcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagccaatcaggagcttcaaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattccgcaatacggctacctgacgctcaacaatggcagccaagccgtgggacgttcatccttttactgcctggaatatttcccttctcagatgctgagaacgggcaacaactttcagttcagctacaccttcgaggacgtgcctttccacagcagctacgcgcacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaacagggacgtgtacctgcagggtcccatctgggccaagattcctcacacggatggcaactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaggtcagcgtggaaatcgagtgggagctgcagaaagaaaacagcaaacgctggaatccagagattcaatacacttccaactactacaaatctacaaatgtggactttgctgtcaacacggagggggtttatactgagcctcgccccattggcacccgttacctcacccgtcccctgtaa

SEQ ID NO: 54 - protein, 735 aa, synthetic construct
MSFVDHPPDWLEDNLSEGVREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTIASGGGAPMADNNEGADGVGNASGNWHCDSTWLGDRVITTSTRTWALPTYNNHLYKQISSQSGASNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPAEFSATKFASFITQYSTGQVSVEIEWELQKENSKRWNPEIQYTSNYYKSTNVDFAVNTEGVYTEPRPIGTRYLTRPL

SEQ ID NO: 55 - DNA, 2208 bp, synthetic construct
  source 1..2208: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgcttcctggctacaagtacctcggacccttcaacggactcgacaagggggagcccgtcaacgaggcggacgcggcagccctcgaacacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaattgcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagccaatcaggagcttcaaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattccgcaatacggctacctgacgctcaacaatggcagccaagccgtgggacgttcatccttttactgcctggaatatttcccttctcagatgctgagaacgggcaacaactttcagttcagctacaccttcgaggacgtgcctttccacagcagctacgcgcacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaacagggacgtgtacctgcagggtcccatctgggccaagattcctcacacggatggcaactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaggtcagcgtggaaatcgagtgggagctgcagaaagaaaacagcaaacgctggaatccagagattcaatacacttccaactactacaaatctacaaatgtggactttgctgtcaacacggagggggtttatactgagcctcgccccattggcacccgttacctcacccgtcccctgtaa

SEQ ID NO: 56 - protein, 735 aa, synthetic construct
MSFVDHPPDWLEEVGEGLREFLGLEAGPPKPKPNQQHQDQARGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTIASGGGAPMADNNEGADGVGNASGNWHCDSTWLGDRVITTSTRTWALPTYNNHLYKQISSQSGASNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPAEFSATKFASFITQYSTGQVSVEIEWELQKENSKRWNPEIQYTSNYYKSTNVDFAVNTEGVYTEPRPIGTRYLTRPL

SEQ ID NO: 57 - DNA, 2208 bp, synthetic construct
  source 1..2208: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgcttcctggctacaagtacctcggacccttcaacggactcgacaagggggagcccgtcaacgaggcggacgcggcagccctcgaacacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaattgcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagccaatcaggagcttcaaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattccgcaatacggctacctgacgctcaacaatggcagccaagccgtgggacgttcatccttttactgcctggaatatttcccttctcagatgctgagaacgggcaacaactttcagttcagctacaccttcgaggacgtgcctttccacagcagctacgcgcacagccagagcctggaccggctgatgaatcccctcatcgaccagtacttgtactacctggccagaacacagagtaacccaggaggcacagctggcaatcgggaactgcagtttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaaactcgttggttaatcccggcgtcgccatggcaactcacaaggacgacgaggaccgcttttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaacagggacgtgtacctgcagggtcccatctgggccaagattcctcacacggatggcaactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaggtcagcgtggaaatcgagtgggagctgcagaaagaaaacagcaaacgctggaatccagagattcaatacacttccaactactacaaatctacaaatgtggactttgctgtcaacacggagggggtttatactgagcctcgccccattggcacccgttacctcacccgtcccctgtaa

SEQ ID NO: 58 - protein, 735 aa, synthetic construct
MSFVDHPPDWLEEVGEGLREFLGLEAGPPKPKPNQQHQDQARGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTIASGGGAPMADNNEGADGVGNASGNWHCDSTWLGDRVITTSTRTWALPTYNNHLYKQISSQSGASNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLARTQSNPGGTAGNRELQFLPGRAFNYGRTSQELVTWTLLPATKSLQNAGSKQQQQLCLDWCHQISPERQKTRWLIPASPWQLTRTTRTAFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPAEFSATKFASFITQYSTGQVSVEIEWELQKENSKRWNPEIQYTSNYYKSTNVDFAVNTEGVYTEPRPIGTRYLTRPL

SEQ ID NO: 59 - DNA, 2208 bp, synthetic construct
  source 1..2208: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgcttcctggctacaagtacctcggacccttcaacggactcgacaagggggagcccgtcaacgaggcggacgcggcagccctcgaacacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaattgcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagccaatcaggagcttcaaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattccgcaatacggctacctgacgctcaacaatggcagccaagccgtgggacgttcatccttttactgcctggaatatttcccttctcagatgctgagaacgggcaacaactttcagttcagctacaccttcgaggacgtgcctttccacagcagctacgcgcacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcaagaaaactcgttggttaatcccggcgtcgccatggcaactcacaaggacgacgaggaccgcttttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaacagggacgtgtacctgcagggtcccatctgggccaagattcctcacacggatggcaactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaggtcagcgtggaaatcgagtgggagctgcagaaagaaaacagcaaacgctggaatccagagattcaatacacttccaactactacaaatctacaaatgtggactttgctgtcaacacggagggggtttatactgagcctcgccccattggcacccgttacctcacccgtcccctgtaa

SEQ ID NO: 60 - protein, 735 aa, synthetic construct
MSFVDHPPDWLEEVGEGLREFLGLEAGPPKPKPNQQHQDQARGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTIASGGGAPMADNNEGADGVGNASGNWHCDSTWLGDRVITTSTRTWALPTYNNHLYKQISSQSGASNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGKKTRWLIPASPWQLTRTTRTAFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPAEFSATKFASFITQYSTGQVSVEIEWELQKENSKRWNPEIQYTSNYYKSTNVDFAVNTEGVYTEPRPIGTRYLTRPL

SEQ ID NO: 61 - DNA, 2208 bp, synthetic construct
  source 1..2208: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacgggctcgatcgaggagagcctgtcaacgaggcggacgcggcagccctcgaacacgacaaagcttacgaccagcagctcaaggccggtgacaacccgtacctcaagtacaaccacgccgacgccgagtttcaggagaagctcgccgacgacacatccttcgggggaaacctcggaaaggcagtctttcaggccaagaaaagggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcagccccctcaggtctgggacctaatacgatggctacaggcagtggcgcaccaatggcagacaataacgaaggcgccgacggagtgggtagttcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgatttcaacagattccactgccatttctcaccacgtgactggcaacgactcatcaacaacaattggggattccggcccaaaagactcaacttcaagctcttcaacatccaagtcaaggaggtcacgacgaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgcacacagccagagcttggaccggctgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacagatggacactttcacccgtctcctcttatgggcggctttggactcaagaacccgcctcctcagatcctcatcaaaaacacgcctgttcccgctaatcctccaactaccttcagtcaagctaagctggcgtcgttcatcacacaatacagcaccggccaggtcgctgttcagattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 62 - protein, 735 aa, synthetic construct
MSFVDHPPDWLEEVGEGLREFLGLEAGPPKPKPNQQHQDQARGLVLPGYNYLGPGNGLDRGEPVNEADAAALEHDKAYDQQLKAGDNPYLKYNHADAEFQEKLADDTSFGGNLGKAVFQAKKRVLEPLGLVEEGAKTAPGKKRPVEQSPQEPDSSSGIGKTGQQPAKKRLNFGQTGDSESVPDPQPLGEPPAAPSGLGPNTMATGSGAPMADNNEGADGVGSSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTTNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEEYGIVADNLQQQNTAPQIGTVNSQGALPGMVWQNRDVYLQGPIWAKIPHTDGHFHPSPLMGGFGLKNPPPQILIKNTPVPANPPTTFSQAKLASFITQYSTGQVAVQIEWELQKENSKRWNPEIQYTSNYYKSTSVDFAVNTEGVYSEPRPIGTRYLTRNL

(19) United States
(12) Patent Application Publication
(10) Pub. No.: US 2026/0325672 A1
(43) Pub. Date: Oct. 22, 2026

(54) ADENO-ASSOCIATED VIRUS CAPSIDS

(71) Applicant: REJUVENATE BIO Inc., San Diego, CA (US)

(72) Inventor: Noah Davidsohn, Brookline, MA (US)

(21) Appl. No.: 19/114,090

(22) PCT Filed: Sep. 21, 2023
(86) PCT No.: PCT/US23/74729

§ 371 Date: Mar. 21, 2025

Related U.S. Application Data
(60) Provisional application No. 63/376,470, filed on Sep. 21, 2022.

(51) Int. Cl. A61K 48/00; C07K 14/005; C12N 15/86

(52) U.S. Cl. CPC A61K 48/0033; C07K 14/005; C12N 15/86; C12N 2750/14122; C12N 2750/14143; C12N 2750/14152

(57) ABSTRACT

Provided herein are recombinant adeno-associated virus (AAV) capsid proteins, compositions (e.g., rAAV) comprising the capsid proteins, nucleic acids encoding the capsid proteins, and methods of making and using the capsid proteins.

### RELATED APPLICATIONS

This application claims priority to U.S. Provisional Patent Application Ser. No. 63/376,470, filed Sep. 21, 2022, the entire disclosure of which is hereby incorporated herein by reference.

### REFERENCE TO SEQUENCE LISTING

This application contains a sequence listing which has been submitted electronically in ST.26 format and is hereby incorporated by reference in its entirety (said ST.26 copy, created on Sep. 19, 2023, is named “203432_SL.xml” and is 145,517 bytes in size).

### BACKGROUND

Adeno-associated viruses (AAVs) are small, non-enveloped viruses of the genus Dependoviruses within the family Parvoviridae. They package a single-stranded DNA genome that is approximately 4.7 kb in length, consisting of rep and cap genes flanked by inverted terminal repeat regions (ITRs). AAVs are an ideal delivery system for gene therapy because AAV infection of cells in culture is non-cytopathic, and natural infection of humans and other animals is silent. Further, because the signals directing AAV replication, genome encapsidation and integration are contained within the inverted terminal repeats (ITRs) of the AAV genome, essentially all of the internal 4.7 kb of the AAV genome (encoding the replication and structural capsid proteins, rep-cap) can be replaced with heterologous nucleic acid sequences, such as a transgene for effecting gene therapy.

Recombinant AAV (rAAV) gene therapy vectors typically consist of a recombinant AAV genome comprising a gene therapy transgene, packaged within an AAV capsid. The AAV capsid, which is formed by an assembly of 60 AAV capsid proteins, interacts with the cell surface of target cells and determines the tropism and transduction efficiency of rAAV. For tissue-specific genetic diseases, it is desirable to have rAAVs that can specifically and efficiently target tissues in need of gene therapy.

Accordingly, there is a need in the art for tissue-specific AAV capsids for use in gene therapy applications.

### SUMMARY

Provided herein are recombinant adeno-associated virus (AAV) capsid proteins, compositions (e.g., rAAV) comprising the capsid proteins, nucleic acids encoding the capsid proteins, and methods of making and using the capsid proteins.

Accordingly, in one aspect, the instant disclosure provides a recombinant adeno-associated virus (AAV) capsid protein comprising: (i) an amino acid sequence having at least 94% sequence identity to amino acids 1-734 of SEQ ID NO: 2, an amino acid sequence having at least 95% sequence identity to amino acids 137-734 of SEQ ID NO: 2, and/or an amino acid sequence having at least 96% sequence identity to amino acids 202-734 of SEQ ID NO: 2: (ii) an amino acid sequence having at least 96% sequence identity to amino acids 1-736 of SEQ ID NO: 4, an amino acid sequence having at least 95% sequence identity to amino acids 138-736 of SEQ ID NO: 4, and/or an amino acid sequence having at least 96% sequence identity to amino acids 204-736 of SEQ ID NO: 4; (iii) an amino acid sequence having at least 94% sequence identity to amino acids 1-734 of SEQ ID NO: 6, an amino acid sequence having at least 96% sequence identity to amino acids 137-734 of SEQ ID NO: 6, and/or an amino acid sequence having at least 97% sequence identity to amino acids 202-734 of SEQ ID NO: 6; (iv) an amino acid sequence having at least 91% sequence identity to amino acids 1-731 of SEQ ID NO: 8, an amino acid sequence having at least 91% sequence identity to amino acids 134-731 of SEQ ID NO: 8, and/or an amino acid sequence having at least 95% sequence identity to amino acids 199-731 of SEQ ID NO: 8; (v) an amino acid sequence having at least 98% sequence identity to amino acids 1-736 of SEQ ID NO: 10, an amino acid sequence having at least 99% sequence identity to amino acids 138-736 of SEQ ID NO: 10, and/or an amino acid sequence having at least 99% sequence identity to amino acids 203-736 of SEQ ID NO: 10; (vi) an amino acid sequence having at least 96% sequence identity to amino acids 1-736 of SEQ ID NO: 12, an amino acid sequence having at least 96% sequence identity to amino acids 137-736 of SEQ ID NO: 12, and/or an amino acid sequence having at least 97% sequence identity to amino acids 203-736 of SEQ ID NO: 12; (vii) an amino acid sequence having at least 96% sequence identity to amino acids 1-736 of SEQ ID NO: 14, an amino acid sequence having at least 95% sequence identity to amino acids 138-736 of SEQ ID NO: 14, and/or an amino acid sequence having at least 96% sequence identity to amino acids 204-736 of SEQ ID NO: 14; (viii) an amino acid sequence having at least 93% sequence identity to amino acids 1-734 of SEQ ID NO: 16, an amino acid sequence having at least 95% sequence identity to amino acids 137-734 of SEQ ID NO: 16, and/or an amino acid sequence having at least 97% sequence identity to amino acids 202-734 of SEQ ID NO: 16; (ix) an amino acid sequence having at least 95% sequence identity to amino acids 1-735 of SEQ ID NO: 18, an amino acid sequence having at least 95% sequence identity to amino acids 138-735 of SEQ ID NO: 18, and/or an amino acid sequence having at least 96% sequence identity to amino acids 203-735 of SEQ ID NO: 18; (x) an amino acid sequence having at least 93% sequence identity to amino acids 1-736 of SEQ ID NO: 20, an amino acid sequence having at least 95% sequence identity to amino acids 137-736 of SEQ ID NO: 20, and/or an amino acid sequence having at least 96% sequence identity to amino acids 202-736 of SEQ ID NO: 20: (xi) an amino acid sequence having at least 96% sequence identity to amino acids 1-736 of SEQ ID NO: 22, an amino acid sequence having at least 97% sequence identity to amino acids 137-736 of SEQ ID NO: 22, and/or an amino acid sequence having at least 99% sequence identity to amino acids 203-736 of SEQ ID NO: 22; (xii) an amino acid sequence having at least 97% sequence identity to amino acids 1-736 of SEQ ID NO: 24, an amino acid sequence having at least 96% sequence identity to amino acids 138-736 of SEQ ID NO: 24, and/or an amino acid sequence having at least 97% sequence identity to amino acids 204-736 of SEQ ID NO: 24; (xiii) an amino acid sequence having at least 99% sequence identity to amino acids 1-735 of SEQ ID NO: 26, an amino acid sequence having at least 99% sequence identity to amino acids 137-735 of SEQ ID NO: 26, and/or an amino acid sequence having at least 99% sequence identity to amino acids 202-735 of SEQ ID NO: 26: (xiv) an amino acid sequence having at least 92% sequence identity to amino acids 1-734 of SEQ ID NO: 28, an amino acid sequence having at least 94% sequence identity to amino acids 137-734 of SEQ ID NO: 28, and/or an amino acid sequence having at least 96% sequence identity to amino acids 202-734 of SEQ ID NO: 28; (xv) an amino acid sequence having at least 92% sequence identity to amino acids 1-734 of SEQ ID NO: 30, an amino acid sequence having at least 94% sequence identity to amino acids 137-734 of SEQ ID NO: 30, and/or an amino acid sequence having at least 96% sequence identity to amino acids 202-734 of SEQ ID NO: 30; (xvi) an amino acid sequence having at least 95% sequence identity to amino acids 1-735 of SEQ ID NO: 32, an amino acid sequence having at least 95% sequence identity to amino acids 137-735 of SEQ ID NO: 32, and/or an amino acid sequence having at least 96% sequence identity to amino acids 202-735 of SEQ ID NO: 32; (xvii) an amino acid sequence having at least 96% sequence identity to amino acids 1-735 of SEQ ID NO: 34, an amino acid sequence having at least 98% sequence identity to amino acids 137-735 of SEQ ID NO: 34, and/or an amino acid sequence having at least 98% sequence identity to amino acids 202-735 of SEQ ID NO: 34: (xviii) an amino acid sequence having at least 94% sequence identity to amino acids 1-735 of SEQ ID NO: 36, an amino acid sequence having at least 96% sequence identity to amino acids 138-735 of SEQ ID NO: 36, and/or an amino acid sequence having at least 97% sequence identity to amino acids 203-735 of SEQ ID NO: 36: (xix) an amino acid sequence having at least 94% sequence identity to amino acids 1-731 of SEQ ID NO: 38, an amino acid sequence having at least 95% sequence identity to amino acids 134-731 of SEQ ID NO: 38, and/or an amino acid sequence having at least 96% sequence identity to amino acids 199-731 of SEQ ID NO: 38; (xx) an amino acid sequence having at least 94% sequence identity to amino acids 1-735 of SEQ ID NO: 40, an amino acid sequence having at least 97% sequence identity to amino acids 137-735 of SEQ ID NO: 40, and/or an amino acid sequence having at least 97% sequence identity to amino acids 203-735 of SEQ ID NO: 40; (xxi) an amino acid sequence having at least 96% sequence identity to amino acids 1-736 of SEQ ID NO: 42, an amino acid sequence having at least 96% sequence identity to amino acids 138-736 of SEQ ID NO: 42, and/or an amino acid sequence having at least 96% sequence identity to amino acids 204-736 of SEQ ID NO: 42; (xxii) an amino acid sequence having at least 94% sequence identity to amino acids 1-736 of SEQ ID NO: 44, an amino acid sequence having at least 96% sequence identity to amino acids 137-736 of SEQ ID NO: 44, and/or an amino acid sequence having at least 97% sequence identity to amino acids 202-736 of SEQ ID NO: 44; (xxiii) an amino acid sequence having at least 94% sequence identity to amino acids 1-736 of SEQ ID NO: 46, an amino acid sequence having at least 96% sequence identity to amino acids 137-736 of SEQ ID NO: 46, and/or an amino acid sequence having at least 97% sequence identity to amino acids 203-736 of SEQ ID NO: 46; (xxiv) an amino acid sequence having at least 94% sequence identity to amino acids 1-734 of SEQ ID NO: 48, an amino acid sequence having at least 95% sequence identity to amino acids 137-734 of SEQ ID NO: 48, and/or an amino acid sequence having at least 97% sequence identity to amino acids 202-734 of SEQ ID NO: 48; (xxv) an amino acid sequence having at least 94% sequence identity to amino acids 1-734 of SEQ ID NO: 50, an amino acid sequence having at least 95% sequence identity to amino acids 137-734 of SEQ ID NO: 50, and/or an amino acid sequence having at least 95% sequence identity to amino acids 202-734 of SEQ ID NO: 50; (xxvi) an amino acid sequence having at least 93% sequence identity to amino acids 1-734 of SEQ ID NO: 52, an amino acid sequence having at least 94% sequence identity to amino acids 137-734 of SEQ ID NO: 52, and/or an amino acid sequence having at least 96% sequence identity to amino acids 202-734 of SEQ ID NO: 52; (xxvii) an amino acid sequence having at least 96% sequence identity to amino acids 1-735 of SEQ ID NO: 54, an amino acid sequence having at least 95% sequence identity to amino acids 138-735 of SEQ ID NO: 54, and/or an amino acid sequence having at least 96% sequence identity to amino acids 203-735 of SEQ ID NO: 54; (xxviii) an amino acid sequence having at least 94% sequence identity to amino acids 1-735 of SEQ ID NO: 56, an amino acid sequence having at least 95% sequence identity to amino acids 137-735 of SEQ ID NO: 56, and/or an amino acid sequence having at least 96% sequence identity to amino acids 202-735 of SEQ ID NO: 56; (xxix) an amino acid sequence having at least 84% sequence identity to amino acids 1-735 of SEQ ID NO: 58, an amino acid sequence having at least 85% sequence identity to amino acids 137-735 of SEQ ID NO: 58, and/or an amino acid sequence having at least 85% sequence identity to amino acids 202-735 of SEQ ID NO: 58; (xxx) an amino acid sequence having at least 90% sequence identity to amino acids 1-735 of SEQ ID NO: 60, an amino acid sequence having at least 92% sequence identity to amino acids 137-735 of SEQ ID NO: 60, and/or an amino acid sequence having at least 93% sequence identity to amino acids 202-735 of SEQ ID NO: 60; or (xxxi) an amino acid sequence having at least 94% sequence identity to amino acids 1-735 of SEQ ID NO: 62, an amino acid sequence having at least 96% sequence identity to amino acids 137-735 of SEQ ID NO: 62, and/or an amino acid sequence having at least 97% sequence identity to amino acids 202-735 of SEQ ID NO: 62.

In certain embodiments, the instant disclosure provides a recombinant AAV capsid protein comprising: (i) an amino acid sequence having at least 99% sequence identity to amino acids 1-734 of SEQ ID NO: 2, an amino acid sequence having at least 99% sequence identity to amino acids 137-734 of SEQ ID NO: 2, and/or an amino acid sequence having at least 99% sequence identity to amino acids 202-734 of SEQ ID NO: 2; (ii) an amino acid sequence having at least 99% sequence identity to amino acids 1-736 of SEQ ID NO: 4, an amino acid sequence having at least 99% sequence identity to amino acids 138-736 of SEQ ID NO: 4, and/or an amino acid sequence having at least 99% sequence identity to amino acids 204-736 of SEQ ID NO: 4; (iii) an amino acid sequence having at least 99% sequence identity to amino acids 1-734 of SEQ ID NO: 6, an amino acid sequence having at least 99% sequence identity to amino acids 137-734 of SEQ ID NO: 6, and/or an amino acid sequence having at least 99% sequence identity to amino acids 202-734 of SEQ ID NO: 6; (iv) an amino acid sequence having at least 99% sequence identity to amino acids 1-731 of SEQ ID NO: 8, an amino acid sequence having at least 99% sequence identity to amino acids 134-731 of SEQ ID NO: 8, and/or an amino acid sequence having at least 99% sequence identity to amino acids 199-731 of SEQ ID NO: 8; (v) an amino acid sequence having at least 99% sequence identity to amino acids 1-736 of SEQ ID NO: 10, an amino acid sequence having at least 99% sequence identity to amino acids 138-736 of SEQ ID NO: 10, and/or an amino acid sequence having at least 99% sequence identity to amino acids 203-736 of SEQ ID NO: 10; (vi) an amino acid sequence having at least 99% sequence identity to amino acids 1-736 of SEQ ID NO: 12, an amino acid sequence having at least 99% sequence identity to amino acids 137-736 of SEQ ID NO: 12, and/or an amino acid sequence having at least 99% sequence identity to amino acids 203-736 of SEQ ID NO: 12; (vii) an amino acid sequence having at least 99% sequence identity to amino acids 1-736 of SEQ ID NO: 14, an amino acid sequence having at least 99% sequence identity to amino acids 138-736 of SEQ ID NO: 14, and/or an amino acid sequence having at least 99% sequence identity to amino acids 204-736 of SEQ ID NO: 14; (viii) an amino acid sequence having at least 99% sequence identity to amino acids 1-734 of SEQ ID NO: 16, an amino acid sequence having at least 99% sequence identity to amino acids 137-734 of SEQ ID NO: 16, and/or an amino acid sequence having at least 99% sequence identity to amino acids 202-734 of SEQ ID NO: 16; (ix) an amino acid sequence having at least 99% sequence identity to amino acids 1-735 of SEQ ID NO: 18, an amino acid sequence having at least 99% sequence identity to amino acids 138-735 of SEQ ID NO: 18, and/or an amino acid sequence having at least 99% sequence identity to amino acids 203-735 of SEQ ID NO: 18; (x) an amino acid sequence having at least 99% sequence identity to amino acids 1-736 of SEQ ID NO: 20, an amino acid sequence having at least 99% sequence identity to amino acids 137-736 of SEQ ID NO: 20, and/or an amino acid sequence having at least 99% sequence identity to amino acids 202-736 of SEQ ID NO: 20: (xi) an amino acid sequence having at least 99% sequence identity to amino acids 1-736 of SEQ ID NO: 22, an amino acid sequence having at least 99% sequence identity to amino acids 137-736 of SEQ ID NO: 22, and/or an amino acid sequence having at least 99% sequence identity to amino acids 203-736 of SEQ ID NO: 22; (xii) an amino acid sequence having at least 99% sequence identity to amino acids 1-736 of SEQ ID NO: 24, an amino acid sequence having at least 99% sequence identity to amino acids 138-736 of SEQ ID NO: 24, and/or an amino acid sequence having at least 99% sequence identity to amino acids 204-736 of SEQ ID NO: 24; (xiii) an amino acid sequence having at least 99% sequence identity to amino acids 1-735 of SEQ ID NO: 26, an amino acid sequence having at least 99% sequence identity to amino acids 137-735 of SEQ ID NO: 26, and/or an amino acid sequence having at least 99% sequence identity to amino acids 202-735 of SEQ ID NO: 26; (xiv) an amino acid sequence having at least 99% sequence identity to amino acids 1-734 of SEQ ID NO: 28, an amino acid sequence having at least 99% sequence identity to amino acids 137-734 of SEQ ID NO: 28, and/or an amino acid sequence having at least 99% sequence identity to amino acids 202-734 of SEQ ID NO: 28; (xv) an amino acid sequence having at least 99% sequence identity to amino acids 1-734 of SEQ ID NO: 30, an amino acid sequence having at least 99% sequence identity to amino acids 137-734 of SEQ ID NO: 30, and/or an amino acid sequence having at least 99% sequence identity to amino acids 202-734 of SEQ ID NO: 30: (xvi) an amino acid sequence having at least 99% sequence identity to amino acids 1-735 of SEQ ID NO: 32, an amino acid sequence having at least 99% sequence identity to amino acids 137-735 of SEQ ID NO: 32, and/or an amino acid sequence having at least 99% sequence identity to amino acids 202-735 of SEQ ID NO: 32; (xvii) an amino acid sequence having at least 99% sequence identity to amino acids 1-735 of SEQ ID NO: 34, an amino acid sequence having at least 99% sequence identity to amino acids 137-735 of SEQ ID NO: 34, and/or an amino acid sequence having at least 99% sequence identity to amino acids 202-735 of SEQ ID NO: 34; (xviii) an amino acid sequence having at least 99% sequence identity to amino acids 1-735 of SEQ ID NO: 36, an amino acid sequence having at least 99% sequence identity to amino acids 138-735 of SEQ ID NO: 36, and/or an amino acid sequence having at least 99% sequence identity to amino acids 203-735 of SEQ ID NO: 36; (xix) an amino acid sequence having at least 99% sequence identity to amino acids 1-731 of SEQ ID NO: 38, an amino acid sequence having at least 99% sequence identity to amino acids 134-731 of SEQ ID NO: 38, and/or an amino acid sequence having at least 99% sequence identity to amino acids 199-731 of SEQ ID NO: 38; (xx) an amino acid sequence having at least 99% sequence identity to amino acids 1-735 of SEQ ID NO: 40, an amino acid sequence having at least 99% sequence identity to amino acids 137-735 of SEQ ID NO: 40, and/or an amino acid sequence having at least 99% sequence identity to amino acids 203-735 of SEQ ID NO: 40: (xxi) an amino acid sequence having at least 99% sequence identity to amino acids 1-736 of SEQ ID NO: 42, an amino acid sequence having at least 99% sequence identity to amino acids 138-736 of SEQ ID NO: 42, and/or an amino acid sequence having at least 99% sequence identity to amino acids 204-736 of SEQ ID NO: 42; (xxii) an amino acid sequence having at least 99% sequence identity to amino acids 1-736 of SEQ ID NO: 44, an amino acid sequence having at least 99% sequence identity to amino acids 137-736 of SEQ ID NO: 44, and/or an amino acid sequence having at least 99% sequence identity to amino acids 202-736 of SEQ ID NO: 44; (xxiii) an amino acid sequence having at least 99% sequence identity to amino acids 1-736 of SEQ ID NO: 46, an amino acid sequence having at least 99% sequence identity to amino acids 137-736 of SEQ ID NO: 46, and/or an amino acid sequence having at least 99% sequence identity to amino acids 203-736 of SEQ ID NO: 46; (xxiv) an amino acid sequence having at least 99% sequence identity to amino acids 1-734 of SEQ ID NO: 48, an amino acid sequence having at least 99% sequence identity to amino acids 137-734 of SEQ ID NO: 48, and/or an amino acid sequence having at least 99% sequence identity to amino acids 202-734 of SEQ ID NO: 48; (xxv) an amino acid sequence having at least 99% sequence identity to amino acids 1-734 of SEQ ID NO: 50, an amino acid sequence having at least 99% sequence identity to amino acids 137-734 of SEQ ID NO: 50, and/or an amino acid sequence having at least 99% sequence identity to amino acids 202-734 of SEQ ID NO: 50; (xxvi) an amino acid sequence having at least 99% sequence identity to amino acids 1-734 of SEQ ID NO: 52, an amino acid sequence having at least 99% sequence identity to amino acids 137-734 of SEQ ID NO: 52, and/or an amino acid sequence having at least 99% sequence identity to amino acids 202-734 of SEQ ID NO: 52; (xxvii) an amino acid sequence having at least 99% sequence identity to amino acids 1-735 of SEQ ID NO: 54, an amino acid sequence having at least 99% sequence identity to amino acids 138-735 of SEQ ID NO: 54, and/or an amino acid sequence having at least 99% sequence identity to amino acids 203-735 of SEQ ID NO: 54; (xxviii) an amino acid sequence having at least 99% sequence identity to amino acids 1-735 of SEQ ID NO: 56, an amino acid sequence having at least 99% sequence identity to amino acids 137-735 of SEQ ID NO: 56, and/or an amino acid sequence having at least 99% sequence identity to amino acids 202-735 of SEQ ID NO: 56; (xxix) an amino acid sequence having at least 99% sequence identity to amino acids 1-735 of SEQ ID NO: 58, an amino acid sequence having at least 99% sequence identity to amino acids 137-735 of SEQ ID NO: 58, and/or an amino acid sequence having at least 99% sequence identity to amino acids 202-735 of SEQ ID NO: 58; (xxx) an amino acid sequence having at least 99% sequence identity to amino acids 1-735 of SEQ ID NO: 60, an amino acid sequence having at least 99% sequence identity to amino acids 137-735 of SEQ ID NO: 60, and/or an amino acid sequence having at least 99% sequence identity to amino acids 202-735 of SEQ ID NO: 60; or (xxxi) an amino acid sequence having at least 99% sequence identity to amino acids 1-735 of SEQ ID NO: 62, an amino acid sequence having at least 99% sequence identity to amino acids 137-735 of SEQ ID NO: 62, and/or an amino acid sequence having at least 99% sequence identity to amino acids 202-735 of SEQ ID NO: 62.

In certain embodiments, the recombinant AAV capsid protein comprises amino acids 1-734 of SEQ ID NO: 2, amino acids 137-734 of SEQ ID NO: 2, and/or amino acids 202-734 of SEQ ID NO: 2. In certain embodiments, the recombinant AAV capsid protein comprises SEQ ID NO: 2.

In certain embodiments, the recombinant AAV capsid protein comprises amino acids 1-736 of SEQ ID NO: 4, amino acids 138-736 of SEQ ID NO: 4, and/or amino acids 204-736 of SEQ ID NO: 4. In certain embodiments, the recombinant AAV capsid protein comprises SEQ ID NO: 4.

In certain embodiments, the recombinant AAV capsid protein comprises amino acids 1-734 of SEQ ID NO: 6, amino acids 137-734 of SEQ ID NO: 6, and/or amino acids 202-734 of SEQ ID NO: 6. In certain embodiments, the recombinant AAV capsid protein comprises SEQ ID NO: 6.

In certain embodiments, the recombinant AAV capsid protein comprises amino acids 1-731 of SEQ ID NO: 8, amino acids 134-731 of SEQ ID NO: 8, and/or amino acids 199-731 of SEQ ID NO: 8. In certain embodiments, the recombinant AAV capsid protein comprises SEQ ID NO: 10.

In certain embodiments, the recombinant AAV capsid protein comprises amino acids 1-736 of SEQ ID NO: 12, amino acids 137-736 of SEQ ID NO: 12, and/or amino acids 203-736 of SEQ ID NO: 12. In certain embodiments, the recombinant AAV capsid protein comprises SEQ ID NO: 12.

In certain embodiments, the recombinant AAV capsid protein comprises amino acids 1-736 of SEQ ID NO: 14, amino acids 138-736 of SEQ ID NO: 14, and/or amino acids 204-736 of SEQ ID NO: 14. In certain embodiments, the recombinant AAV capsid protein comprises SEQ ID NO: 14.

In certain embodiments, the recombinant AAV capsid protein comprises amino acids 1-734 of SEQ ID NO: 16, amino acids 137-734 of SEQ ID NO: 16, and/or amino acids 202-734 of SEQ ID NO: 16. In certain embodiments, the recombinant AAV capsid protein comprises SEQ ID NO: 16.

In certain embodiments, the recombinant AAV capsid protein comprises amino acids 1-735 of SEQ ID NO: 18, amino acids 138-735 of SEQ ID NO: 18, and/or amino acids 203-735 of SEQ ID NO: 18. In certain embodiments, the recombinant AAV capsid protein comprises SEQ ID NO: 18.

In certain embodiments, the recombinant AAV capsid protein comprises amino acids 1-736 of SEQ ID NO: 20, amino acids 137-736 of SEQ ID NO: 20, and/or amino acids 202-736 of SEQ ID NO: 20. In certain embodiments, the recombinant AAV capsid protein comprises SEQ ID NO: 20.

In certain embodiments, the recombinant AAV capsid protein comprises amino acids 1-736 of SEQ ID NO: 22, amino acids 137-736 of SEQ ID NO: 22, and/or amino acids 203-736 of SEQ ID NO: 22. In certain embodiments, the recombinant AAV capsid protein comprises SEQ ID NO: 22.

In certain embodiments, the recombinant AAV capsid protein comprises amino acids 1-736 of SEQ ID NO: 24, amino acids 138-736 of SEQ ID NO: 24, and/or amino acids 204-736 of SEQ ID NO: 24. In certain embodiments, the recombinant AAV capsid protein comprises SEQ ID NO: 24.

In certain embodiments, the recombinant AAV capsid protein comprises amino acids 1-735 of SEQ ID NO: 26, amino acids 137-735 of SEQ ID NO: 26, and/or amino acids 202-735 of SEQ ID NO: 26. In certain embodiments, the recombinant AAV capsid protein comprises SEQ ID NO: 26.

In certain embodiments, the recombinant AAV capsid protein comprises amino acids 1-734 of SEQ ID NO: 28, amino acids 137-734 of SEQ ID NO: 28, and/or amino acids 202-734 of SEQ ID NO: 28. In certain embodiments, the recombinant AAV capsid protein comprises SEQ ID NO: 28.

In certain embodiments, the recombinant AAV capsid protein comprises amino acids 1-734 of SEQ ID NO: 30, amino acids 137-734 of SEQ ID NO: 30, and/or amino acids 202-734 of SEQ ID NO: 30. In certain embodiments, the recombinant AAV capsid protein comprises SEQ ID NO: 30.

In certain embodiments, the recombinant AAV capsid protein comprises amino acids 1-735 of SEQ ID NO: 32, amino acids 137-735 of SEQ ID NO: 32, and/or amino acids 202-735 of SEQ ID NO: 32. In certain embodiments, the recombinant AAV capsid protein comprises SEQ ID NO: 32.

In certain embodiments, the recombinant AAV capsid protein comprises amino acids 1-735 of SEQ ID NO: 34, amino acids 137-735 of SEQ ID NO: 34, and/or amino acids 202-735 of SEQ ID NO: 34. In certain embodiments, the recombinant AAV capsid protein comprises SEQ ID NO: 34.

In certain embodiments, the recombinant AAV capsid protein comprises amino acids 1-735 of SEQ ID NO: 36, amino acids 138-735 of SEQ ID NO: 36, and/or amino acids 203-735 of SEQ ID NO: 36. In certain embodiments, the recombinant AAV capsid protein comprises SEQ ID NO: 36.

In certain embodiments, the recombinant AAV capsid protein comprises amino acids 1-731 of SEQ ID NO: 38, amino acids 134-731 of SEQ ID NO: 38, and/or amino acids 199-731 of SEQ ID NO: 38. In certain embodiments, the recombinant AAV capsid protein comprises SEQ ID NO: 38.

In certain embodiments, the recombinant AAV capsid protein comprises amino acids 1-735 of SEQ ID NO: 40, amino acids 137-735 of SEQ ID NO: 40, and/or amino acids 203-735 of SEQ ID NO: 40. In certain embodiments, the recombinant AAV capsid protein comprises SEQ ID NO: 40.

In certain embodiments, the recombinant AAV capsid protein comprises amino acids 1-736 of SEQ ID NO: 42, amino acids 138-736 of SEQ ID NO: 42, and/or amino acids 204-736 of SEQ ID NO: 42. In certain embodiments, the recombinant AAV capsid protein comprises SEQ ID NO: 42.

In certain embodiments, the recombinant AAV capsid protein comprises amino acids 1-736 of SEQ ID NO: 44, amino acids 137-736 of SEQ ID NO: 44, and/or amino acids 202-736 of SEQ ID NO: 44. In certain embodiments, the recombinant AAV capsid protein comprises SEQ ID NO: 44.

In certain embodiments, the recombinant AAV capsid protein comprises amino acids 1-736 of SEQ ID NO: 46, amino acids 137-736 of SEQ ID NO: 46, and/or amino acids 203-736 of SEQ ID NO: 46. In certain embodiments, the recombinant AAV capsid protein comprises SEQ ID NO: 46.

In certain embodiments, the recombinant AAV capsid protein comprises amino acids 1-734 of SEQ ID NO: 48, amino acids 137-734 of SEQ ID NO: 48, and/or amino acids 202-734 of SEQ ID NO: 48. In certain embodiments, the recombinant AAV capsid protein comprises SEQ ID NO: 48.

In certain embodiments, the recombinant AAV capsid protein comprises amino acids 1-734 of SEQ ID NO: 50, amino acids 137-734 of SEQ ID NO: 50, and/or amino acids 202-734 of SEQ ID NO: 50. In certain embodiments, the recombinant AAV capsid protein comprises SEQ ID NO: 50.

In certain embodiments, the recombinant AAV capsid protein comprises amino acids 1-734 of SEQ ID NO: 52, amino acids 137-734 of SEQ ID NO: 52, and/or amino acids 202-734 of SEQ ID NO: 52. In certain embodiments, the recombinant AAV capsid protein comprises SEQ ID NO: 52.

In certain embodiments, the recombinant AAV capsid protein comprises amino acids 1-735 of SEQ ID NO: 54, amino acids 138-735 of SEQ ID NO: 54, and/or amino acids 203-735 of SEQ ID NO: 54. In certain embodiments, the recombinant AAV capsid protein comprises SEQ ID NO: 54.

In certain embodiments, the recombinant AAV capsid protein comprises amino acids 1-735 of SEQ ID NO: 56, amino acids 137-735 of SEQ ID NO: 56, and/or amino acids 202-735 of SEQ ID NO: 56. In certain embodiments, the recombinant AAV capsid protein comprises SEQ ID NO: 56.

In certain embodiments, the recombinant AAV capsid protein comprises amino acids 1-735 of SEQ ID NO: 58, amino acids 137-735 of SEQ ID NO: 58, and/or amino acids 202-735 of SEQ ID NO: 58. In certain embodiments, the recombinant AAV capsid protein comprises SEQ ID NO: 58.

In certain embodiments, the recombinant AAV capsid protein comprises amino acids 1-735 of SEQ ID NO: 60, amino acids 137-735 of SEQ ID NO: 60, and/or amino acids 202-735 of SEQ ID NO: 60. In certain embodiments, the recombinant AAV capsid protein comprises SEQ ID NO: 60.

In certain embodiments, the recombinant AAV capsid protein comprises amino acids 1-735 of SEQ ID NO: 62, amino acids 137-735 of SEQ ID NO: 62, and/or amino acids 202-735 of SEQ ID NO: 62. In certain embodiments, the recombinant AAV capsid protein comprises SEQ ID NO: 62.

In certain embodiments, the recombinant AAV capsid protein of the present disclosure is a variant of a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAVS capsid protein.

In another aspect, the instant disclosure provides an isolated polynucleotide encoding a recombinant AAV capsid protein as described in the instant disclosure. In certain embodiments, the isolated polynucleotide has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the nucleic acid sequence set forth in any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, and 61. In certain embodiments, the isolated polynucleotide comprises the nucleic acid sequence set forth in any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, and 61.

In another aspect, the instant disclosure provides a vector comprising an isolated polynucleotide as described in the instant disclosure. In certain embodiments, the vector is a plasmid or a viral vector. In certain embodiments, the viral vector is a retrovirus vector, a herpes virus vector, a baculovirus vector, or an adenovirus vector. In certain embodiments, the vector is an expression vector.

In another aspect, the instant disclosure provides a recombinant cell comprising a polynucleotide as described in the instant disclosure or a vector as described in the instant disclosure.

In another aspect, the instant disclosure provides a method of producing a recombinant AAV capsid protein, the method comprising culturing a recombinant cell as described in the instant disclosure under conditions where the polynucleotide is expressed, and the capsid protein is produced.

In another aspect, the instant disclosure provides a recombinant adeno-associated virus (rAAV) comprising: (a) a capsid comprising one or more of the recombinant AAV capsid proteins of the instant disclosure; and (b) an rAAV genome. In certain embodiments, the rAAV genome comprises a transgene. In certain embodiments, the transgene encodes a polypeptide. In certain embodiments, the transgene encodes a miRNA, shRNA, siRNA, antisense RNA, gRNA, antagomir, miRNA sponge, RNA aptazyme, RNA aptamer, lncRNA, ribozyme or mRNA. In certain embodiments, the transgene is operably linked to a transcriptional regulatory element.

In another aspect, the instant disclosure provides a method for transducing a cell, the method comprising contacting the cell with a rAAV of the instant disclosure under conditions whereby the cell is transduced. In certain embodiments, the instant disclosure provides a method for delivering a transgene in a cell, the method comprising contacting the cell with a rAAV of the instant disclosure under conditions whereby the cell is transduced and the transgene is expressed. In certain embodiments, the cell is in a subject and the rAAV is administered to the subject. In certain embodiments, the cell is a muscle cell and/or a liver cell. In certain embodiments, the rAAV is administered to the subject intravenously, intraperitoneally, subcutaneously, intramuscularly, intrathecally, or intradermally. In certain embodiments, the subject is selected from the group consisting of a human subject, a canine subject, a feline subject, and an equine subject. In certain embodiments, the subject is a human subject.

In another aspect, the instant disclosure provides a recombinant AAV comprising: (a) a capsid comprising one or more of the recombinant AAV capsid proteins of the instant disclosure; and (b) an rAAV genome, for use in medicine, for use as therapy, or for use as a medicament.

In another aspect, the instant disclosure provides a packaging system for preparation of an rAAV, wherein the packaging system comprises: (a) a first nucleotide sequence encoding one or more AAV Rep proteins; (b) a second nucleotide sequence encoding a recombinant AAV capsid protein of the instant disclosure; and (c) a third nucleotide sequence comprising an rAAV genome sequence. In certain embodiments, the packaging system comprises a first vector comprising the first nucleotide sequence and the second nucleotide sequence, and a second vector comprising the third nucleotide sequence.

In certain embodiments, the packaging system of the instant disclosure further comprises a fourth nucleotide sequence comprising one or more helper virus genes. In certain embodiments, the fourth nucleotide sequence is comprised within a third vector. In certain embodiments, the fourth nucleotide sequence comprises one or more genes from a virus selected from the group consisting of adenovirus, herpesvirus, vaccinia virus, and cytomegalovirus (CMV).

In certain embodiments, the first vector, second vector, and/or the third vector of the packaging system of the instant disclosure is a plasmid.

In another aspect, the instant disclosure provides a method for recombinant preparation of an rAAV, the method comprising introducing the packaging system as described in the instant disclosure into a cell under conditions whereby the rAAV is produced.

### BRIEF DESCRIPTION OF THE DRAWINGS

FIG. 1 shows the expression level of FGF21 (FIG. 1A) or sTGFbR2 (FIG. 1B) in plasma samples obtained from dogs intravenously administered 5×10¹² vg/kg of vector genomes comprising FGF21 and sTGFbR2 coding sequences packaged in CL01, CL03, CL11, CL13, CL27, or AAV8 (control) capsids. FIG. 1A shows the expression of FGF21 as assessed by ELISA and FIG. 1B shows the expression of sTGFbR2 as assessed by Western blot.

### DETAILED DESCRIPTION

Provided herein are recombinant adeno-associated virus (AAV) capsid proteins, compositions (e.g., rAAV) comprising the capsid proteins, nucleic acids encoding the capsid proteins, and methods of making and using the capsid proteins. The AAV capsid proteins described herein have been isolated from screening a shuffled capsid library in vivo for AAVs that are enriched in the liver and/or muscle.

### I. DEFINITIONS

As used herein, the term “AAV” is a standard abbreviation for adeno-associated virus.

As used herein interchangeably, the terms “recombinant adeno-associated virus” or “rAAV” refer to an AAV comprising a genome lacking functional rep and cap genes.

As used herein, the term “cap gene” refers to a nucleic acid sequence that encodes a capsid protein. For AAV, the capsid protein may be VP1, VP2, or VP3. VP1, VP2, and/or VP3 capsid proteins assemble into a capsid that surrounds the rAAV genome.

As used herein, the term “rep gene” refers to the nucleic acid sequences that encode the non-structural proteins (e.g., rep78, rep68, rep52, and rep40) required for the replication and production of an AAV.

As used herein, the term “rAAV genome” refers to a nucleic acid molecule (e.g., DNA and/or RNA) comprising the genome sequence of an rAAV. The skilled artisan will appreciate that where an rAAV genome comprises a transgene (e.g., a polypeptide encoding a therapeutic protein operably linked to a transcriptional regulatory element (i.e., payload)), the rAAV genome can be in the sense or antisense orientation relative to the direction of transcription of the transgene.

As used herein, an “isolated polynucleotide” refers to a polynucleotide that has been separated from one or more nucleic acid molecules present in the natural source of the polynucleotide.

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches, and does not consider the degree of similarity of amino acids to one another. Note that only internal gaps are included in the length, not gaps at the sequence ends.

As used herein, a “vector” refers to a nucleic acid molecule that is a vehicle for introducing a nucleic acid molecule (e.g., a polynucleotide disclosed herein) into a cell.

As used herein, an “expression vector” refers to a vector comprising transcriptional regulatory elements operably linked to a gene of interest (e.g., a polynucleotide disclosed herein) that facilitate the expression of the gene of interest in a cell and/or a cell free expression system.

As used herein, the term “transgene” refers to a non-AAV nucleic acid sequence that encodes a polypeptide (e.g., an antibody or scFv) or non-coding RNA (e.g., an miRNA, shRNA, siRNA, antisense RNA, gRNA, antagomir, miRNA sponge, RNA aptazyme, or RNA aptamer).

As used herein, the term “transcriptional regulatory element” or “TRE” refers to a cis-acting nucleotide sequence, for example, a DNA sequence, that regulates (e.g., controls, increases, or reduces) transcription of an operably linked nucleotide sequence by an RNA polymerase to form an RNA molecule. A TRE may comprise one or more promoter elements and/or enhancer elements. A skilled artisan would appreciate that the promoter and enhancer elements in a gene may be close in location, and the term “promoter” may refer to a sequence comprising a promoter element and an enhancer element. Thus, the term “promoter” does not exclude an enhancer element in the sequence. The promoter and enhancer elements do not need to be derived from the same gene or species, and the sequence of each promoter or enhancer element may be either identical or substantially identical to the corresponding endogenous sequence in the genome.

As used herein, the term “operably linked” is used to describe the connection between a TRE and a polynucleotide sequence (e.g., a transgene disclosed herein) to be transcribed. Typically, gene expression is placed under the control of a TRE comprising one or more promoter and/or enhancer elements. The transgene is “operably linked” to the TRE if the transcription of the transgene is controlled or influenced by the TRE. The promoter and enhancer elements of the TRE may be in any orientation and/or distance from the transgene, as long as the desired transcriptional activity is obtained. In an embodiment, the TRE is upstream from the transgene.

As used herein, the term “effective amount” in the context of the administration of an AAV to a subject refers to the amount of the AAV that achieves a desired prophylactic or therapeutic effect.

### II. AAV CAPSID PROTEINS

In one aspect, the present disclosure provides a recombinant adeno-associated virus (AAV) capsid protein. The AAV capsid proteins described herein have been isolated from screening a shuffled capsid library in vivo for AAVs that are enriched in the liver and/or muscle.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 2. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 2. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 2.

In certain embodiments, the recombinant AAV capsid protein comprises amino acids 1-734 of SEQ ID NO: 2. In certain embodiments, the recombinant AAV capsid protein comprises amino acids 137-734 of SEQ ID NO: 2. In certain embodiments, the recombinant AAV capsid protein comprises amino acids 202-734 of SEQ ID NO: 2. In certain embodiments, the recombinant AAV capsid protein comprises SEQ ID NO: 2.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 4. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-736 of SEQ ID NO: 4. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 204-736 of SEQ ID NO: 4.

In certain embodiments, the recombinant AAV capsid protein comprises amino acids 1-736 of SEQ ID NO: 4. In certain embodiments, the recombinant AAV capsid protein comprises amino acids 138-736 of SEQ ID NO: 4. In certain embodiments, the recombinant AAV capsid protein comprises amino acids 204-736 of SEQ ID NO: 4. In certain embodiments, the recombinant AAV capsid protein comprises SEQ ID NO: 4.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 6. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 6. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 6.

In certain embodiments, the recombinant AAV capsid protein comprises amino acids 1-734 of SEQ ID NO: 6. In certain embodiments, the recombinant AAV capsid protein comprises amino acids 137-734 of SEQ ID NO: 6. In certain embodiments, the recombinant AAV capsid protein comprises amino acids 202-734 of SEQ ID NO: 6. In certain embodiments, the recombinant AAV capsid protein comprises SEQ ID NO: 6.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to amino acids 1-731 of SEQ ID NO: 8. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 134-731 of SEQ ID NO: 8. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 199-731 of SEQ ID NO: 8.

In certain embodiments, the recombinant AAV capsid protein comprises amino acids 1-731 of SEQ ID NO: 8. In certain embodiments, the recombinant AAV capsid protein comprises amino acids 134-731 of SEQ ID NO: 8. In certain embodiments, the recombinant AAV capsid protein comprises amino acids 199-731 of SEQ ID NO: 8. In certain embodiments, the recombinant AAV capsid protein comprises SEQ ID NO: 8.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 10. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-736 of SEQ ID NO: 10. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-736 of SEQ ID NO: 10.

In certain embodiments, the recombinant AAV capsid protein comprises amino acids 1-736 of SEQ ID NO: 10. In certain embodiments, the recombinant AAV capsid protein comprises amino acids 138-736 of SEQ ID NO: 10. In certain embodiments, the recombinant AAV capsid protein comprises amino acids 203-736 of SEQ ID NO: 10. In certain embodiments, the recombinant AAV capsid protein comprises SEQ ID NO: 10.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 12. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-736 of SEQ ID NO: 12. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-736 of SEQ ID NO: 12.

In certain embodiments, the recombinant AAV capsid protein comprises amino acids 1-736 of SEQ ID NO: 12. In certain embodiments, the recombinant AAV capsid protein comprises amino acids 137-736 of SEQ ID NO: 12. In certain embodiments, the recombinant AAV capsid protein comprises amino acids 203-736 of SEQ ID NO: 12. In certain embodiments, the recombinant AAV capsid protein comprises SEQ ID NO: 12.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 14. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-736 of SEQ ID NO: 14. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 204-736 of SEQ ID NO: 14.

In certain embodiments, the recombinant AAV capsid protein comprises amino acids 1-736 of SEQ ID NO: 14. In certain embodiments, the recombinant AAV capsid protein comprises amino acids 138-736 of SEQ ID NO: 14. In certain embodiments, the recombinant AAV capsid protein comprises amino acids 204-736 of SEQ ID NO: 14. In certain embodiments, the recombinant AAV capsid protein comprises SEQ ID NO: 14.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 16. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 16. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 16.

In certain embodiments, the recombinant AAV capsid protein comprises amino acids 1-734 of SEQ ID NO: 16. In certain embodiments, the recombinant AAV capsid protein comprises amino acids 137-734 of SEQ ID NO: 16. In certain embodiments, the recombinant AAV capsid protein comprises amino acids 202-734 of SEQ ID NO: 16. In certain embodiments, the recombinant AAV capsid protein comprises SEQ ID NO: 16.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 18. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-735 of SEQ ID NO: 18. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-735 of SEQ ID NO: 18.

In certain embodiments, the recombinant AAV capsid protein comprises amino acids 1-735 of SEQ ID NO: 18. In certain embodiments, the recombinant AAV capsid protein comprises amino acids 138-735 of SEQ ID NO: 18. In certain embodiments, the recombinant AAV capsid protein comprises amino acids 203-735 of SEQ ID NO: 18. In certain embodiments, the recombinant AAV capsid protein comprises SEQ ID NO: 18.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 20. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-736 of SEQ ID NO: 20. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-736 of SEQ ID NO: 20.

In certain embodiments, the recombinant AAV capsid protein comprises amino acids 1-736 of SEQ ID NO: 20. In certain embodiments, the recombinant AAV capsid protein comprises amino acids 137-736 of SEQ ID NO: 20. In certain embodiments, the recombinant AAV capsid protein comprises amino acids 202-736 of SEQ ID NO: 20. In certain embodiments, the recombinant AAV capsid protein comprises SEQ ID NO: 20.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 22. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-736 of SEQ ID NO: 22. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-736 of SEQ ID NO: 22.

In certain embodiments, the recombinant AAV capsid protein comprises amino acids 1-736 of SEQ ID NO: 22. In certain embodiments, the recombinant AAV capsid protein comprises amino acids 137-736 of SEQ ID NO: 22. In certain embodiments, the recombinant AAV capsid protein comprises amino acids 203-736 of SEQ ID NO: 22. In certain embodiments, the recombinant AAV capsid protein comprises SEQ ID NO: 22.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 24. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-736 of SEQ ID NO: 24. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 204-736 of SEQ ID NO: 24.

In certain embodiments, the recombinant AAV capsid protein comprises amino acids 1-736 of SEQ ID NO: 24. In certain embodiments, the recombinant AAV capsid protein comprises amino acids 138-736 of SEQ ID NO: 24. In certain embodiments, the recombinant AAV capsid protein comprises amino acids 204-736 of SEQ ID NO: 24. In certain embodiments, the recombinant AAV capsid protein comprises SEQ ID NO: 24.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 26. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 26. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 26.

In certain embodiments, the recombinant AAV capsid protein comprises amino acids 1-735 of SEQ ID NO: 26. In certain embodiments, the recombinant AAV capsid protein comprises amino acids 137-735 of SEQ ID NO: 26. In certain embodiments, the recombinant AAV capsid protein comprises amino acids 202-735 of SEQ ID NO: 26. In certain embodiments, the recombinant AAV capsid protein comprises SEQ ID NO: 26.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 28. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 28. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 28.

In certain embodiments, the recombinant AAV capsid protein comprises amino acids 1-734 of SEQ ID NO: 28. In certain embodiments, the recombinant AAV capsid protein comprises amino acids 137-734 of SEQ ID NO: 28. In certain embodiments, the recombinant AAV capsid protein comprises amino acids 202-734 of SEQ ID NO: 28. In certain embodiments, the recombinant AAV capsid protein comprises SEQ ID NO: 28.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 30. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 30. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 30.

In certain embodiments, the recombinant AAV capsid protein comprises amino acids 1-734 of SEQ ID NO: 30. In certain embodiments, the recombinant AAV capsid protein comprises amino acids 137-734 of SEQ ID NO: 30. In certain embodiments, the recombinant AAV capsid protein comprises amino acids 202-734 of SEQ ID NO: 30. In certain embodiments, the recombinant AAV capsid protein comprises SEQ ID NO: 30.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 32. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 32. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 32.

In certain embodiments, the recombinant AAV capsid protein comprises amino acids 1-735 of SEQ ID NO: 32. In certain embodiments, the recombinant AAV capsid protein comprises amino acids 137-735 of SEQ ID NO: 32. In certain embodiments, the recombinant AAV capsid protein comprises amino acids 202-735 of SEQ ID NO: 32. In certain embodiments, the recombinant AAV capsid protein comprises SEQ ID NO: 32.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 34. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 34. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 34.

In certain embodiments, the recombinant AAV capsid protein comprises amino acids 1-735 of SEQ ID NO: 34. In certain embodiments, the recombinant AAV capsid protein comprises amino acids 137-735 of SEQ ID NO: 34. In certain embodiments, the recombinant AAV capsid protein comprises amino acids 202-735 of SEQ ID NO: 34. In certain embodiments, the recombinant AAV capsid protein comprises SEQ ID NO: 34.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 36. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-735 of SEQ ID NO: 36. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-735 of SEQ ID NO: 36.

In certain embodiments, the recombinant AAV capsid protein comprises amino acids 1-735 of SEQ ID NO: 36. In certain embodiments, the recombinant AAV capsid protein comprises amino acids 138-735 of SEQ ID NO: 36. In certain embodiments, the recombinant AAV capsid protein comprises amino acids 203-735 of SEQ ID NO: 36. In certain embodiments, the recombinant AAV capsid protein comprises SEQ ID NO: 36.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-731 of SEQ ID NO: 38. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 134-731 of SEQ ID NO: 38. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 199-731 of SEQ ID NO: 38.

In certain embodiments, the recombinant AAV capsid protein comprises amino acids 1-731 of SEQ ID NO: 38. In certain embodiments, the recombinant AAV capsid protein comprises amino acids 134-731 of SEQ ID NO: 38. In certain embodiments, the recombinant AAV capsid protein comprises amino acids 199-731 of SEQ ID NO: 38. In certain embodiments, the recombinant AAV capsid protein comprises SEQ ID NO: 38.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 40. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 40. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-735 of SEQ ID NO: 40.

In certain embodiments, the recombinant AAV capsid protein comprises amino acids 1-735 of SEQ ID NO: 40. In certain embodiments, the recombinant AAV capsid protein comprises amino acids 137-735 of SEQ ID NO: 40. In certain embodiments, the recombinant AAV capsid protein comprises amino acids 203-735 of SEQ ID NO: 40. In certain embodiments, the recombinant AAV capsid protein comprises SEQ ID NO: 40.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 42. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-736 of SEQ ID NO: 42. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 204-736 of SEQ ID NO: 42.

In certain embodiments, the recombinant AAV capsid protein comprises amino acids 1-736 of SEQ ID NO: 42. In certain embodiments, the recombinant AAV capsid protein comprises amino acids 138-736 of SEQ ID NO: 42. In certain embodiments, the recombinant AAV capsid protein comprises amino acids 204-736 of SEQ ID NO: 42. In certain embodiments, the recombinant AAV capsid protein comprises SEQ ID NO: 42.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 44. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-736 of SEQ ID NO: 44. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-736 of SEQ ID NO: 44.

In certain embodiments, the recombinant AAV capsid protein comprises amino acids 1-736 of SEQ ID NO: 44. In certain embodiments, the recombinant AAV capsid protein comprises amino acids 137-736 of SEQ ID NO: 44. In certain embodiments, the recombinant AAV capsid protein comprises amino acids 202-736 of SEQ ID NO: 44. In certain embodiments, the recombinant AAV capsid protein comprises SEQ ID NO: 44.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 46. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-736 of SEQ ID NO: 46. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-736 of SEQ ID NO: 46.

In certain embodiments, the recombinant AAV capsid protein comprises amino acids 1-736 of SEQ ID NO: 46. In certain embodiments, the recombinant AAV capsid protein comprises amino acids 137-736 of SEQ ID NO: 46. In certain embodiments, the recombinant AAV capsid protein comprises amino acids 203-736 of SEQ ID NO: 46. In certain embodiments, the recombinant AAV capsid protein comprises SEQ ID NO: 46.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 48. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 48. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 48.

In certain embodiments, the recombinant AAV capsid protein comprises amino acids 1-734 of SEQ ID NO: 48. In certain embodiments, the recombinant AAV capsid protein comprises amino acids 137-734 of SEQ ID NO: 48. In certain embodiments, the recombinant AAV capsid protein comprises amino acids 202-734 of SEQ ID NO: 48. In certain embodiments, the recombinant AAV capsid protein comprises SEQ ID NO: 48.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 50. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 50. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 50.

In certain embodiments, the recombinant AAV capsid protein comprises amino acids 1-734 of SEQ ID NO: 50. In certain embodiments, the recombinant AAV capsid protein comprises amino acids 137-734 of SEQ ID NO: 50. In certain embodiments, the recombinant AAV capsid protein comprises amino acids 202-734 of SEQ ID NO: 50. In certain embodiments, the recombinant AAV capsid protein comprises SEQ ID NO: 50.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 52. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 52. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 52.

In certain embodiments, the recombinant AAV capsid protein comprises amino acids 1-734 of SEQ ID NO: 52. In certain embodiments, the recombinant AAV capsid protein comprises amino acids 137-734 of SEQ ID NO: 52. In certain embodiments, the recombinant AAV capsid protein comprises amino acids 202-734 of SEQ ID NO: 52. In certain embodiments, the recombinant AAV capsid protein comprises SEQ ID NO: 52.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 54. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-735 of SEQ ID NO: 54. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-735 of SEQ ID NO: 54.

In certain embodiments, the recombinant AAV capsid protein comprises amino acids 1-735 of SEQ ID NO: 54. In certain embodiments, the recombinant AAV capsid protein comprises amino acids 138-735 of SEQ ID NO: 54. In certain embodiments, the recombinant AAV capsid protein comprises amino acids 203-735 of SEQ ID NO: 54. In certain embodiments, the recombinant AAV capsid protein comprises SEQ ID NO: 54.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 56. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 56. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 56.

In certain embodiments, the recombinant AAV capsid protein comprises amino acids 1-735 of SEQ ID NO: 56. In certain embodiments, the recombinant AAV capsid protein comprises amino acids 137-735 of SEQ ID NO: 56. In certain embodiments, the recombinant AAV capsid protein comprises amino acids 202-735 of SEQ ID NO: 56. In certain embodiments, the recombinant AAV capsid protein comprises SEQ ID NO: 56.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 58. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 58. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 58.

In certain embodiments, the recombinant AAV capsid protein comprises amino acids 1-735 of SEQ ID NO: 58. In certain embodiments, the recombinant AAV capsid protein comprises amino acids 137-735 of SEQ ID NO: 58. In certain embodiments, the recombinant AAV capsid protein comprises amino acids 202-735 of SEQ ID NO: 58. In certain embodiments, the recombinant AAV capsid protein comprises SEQ ID NO: 58.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 60. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 60. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 60.

In certain embodiments, the recombinant AAV capsid protein comprises amino acids 1-735 of SEQ ID NO: 60. In certain embodiments, the recombinant AAV capsid protein comprises amino acids 137-735 of SEQ ID NO: 60. In certain embodiments, the recombinant AAV capsid protein comprises amino acids 202-735 of SEQ ID NO: 60. In certain embodiments, the recombinant AAV capsid protein comprises SEQ ID NO: 60.

In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 62. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 62. In certain embodiments, the recombinant AAV capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 62.

In certain embodiments, the recombinant AAV capsid protein comprises amino acids 1-735 of SEQ ID NO: 62. In certain embodiments, the recombinant AAV capsid protein comprises amino acids 137-735 of SEQ ID NO: 62. In certain embodiments, the recombinant AAV capsid protein comprises amino acids 202-735 of SEQ ID NO: 62. In certain embodiments, the recombinant AAV capsid protein comprises SEQ ID NO: 62.

In certain embodiments, the recombinant AAV capsid protein is a variant of a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein.

### III. POLYNUCLEOTIDES, VECTORS, AND METHODS OF PRODUCING AAV CAPSIDS

In another aspect, the present disclosure provides an isolated polynucleotide encoding one or more recombinant AAV capsid proteins as disclosed herein.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 2. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 2. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 2.

In certain embodiments, the isolated polynucleotide encodes amino acids 1-734 of SEQ ID NO: 2. In certain embodiments, the isolated polynucleotide encodes amino acids 137-734 of SEQ ID NO: 2. In certain embodiments, the isolated polynucleotide encodes amino acids 202-734 of SEQ ID NO: 2. In certain embodiments, the isolated polynucleotide encodes SEQ ID NO: 2.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 4. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-736 of SEQ ID NO: 4. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 204-736 of SEQ ID NO: 4.

In certain embodiments, the isolated polynucleotide encodes amino acids 1-736 of SEQ ID NO: 4. In certain embodiments, the isolated polynucleotide encodes amino acids 138-736 of SEQ ID NO: 4. In certain embodiments, the isolated polynucleotide encodes amino acids 204-736 of SEQ ID NO: 4. In certain embodiments, the isolated polynucleotide encodes SEQ ID NO: 4.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 6. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 6. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 6.

In certain embodiments, the isolated polynucleotide encodes amino acids 1-734 of SEQ ID NO: 6. In certain embodiments, the isolated polynucleotide encodes amino acids 137-734 of SEQ ID NO: 6. In certain embodiments, the isolated polynucleotide encodes amino acids 202-734 of SEQ ID NO: 6. In certain embodiments, the isolated polynucleotide encodes SEQ ID NO: 6.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to amino acids 1-731 of SEQ ID NO: 8. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 134-731 of SEQ ID NO: 8. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 199-731 of SEQ ID NO: 8.

In certain embodiments, the isolated polynucleotide encodes amino acids 1-731 of SEQ ID NO: 8. In certain embodiments, the isolated polynucleotide encodes amino acids 134-731 of SEQ ID NO: 8. In certain embodiments, the isolated polynucleotide encodes amino acids 199-731 of SEQ ID NO: 8. In certain embodiments, the isolated polynucleotide encodes SEQ ID NO: 8.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 10. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-736 of SEQ ID NO: 10. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-736 of SEQ ID NO: 10.

In certain embodiments, the isolated polynucleotide encodes amino acids 1-736 of SEQ ID NO: 10. In certain embodiments, the isolated polynucleotide encodes amino acids 138-736 of SEQ ID NO: 10. In certain embodiments, the isolated polynucleotide encodes amino acids 203-736 of SEQ ID NO: 10. In certain embodiments, the isolated polynucleotide encodes SEQ ID NO: 10.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 12. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-736 of SEQ ID NO: 12. In certain embodiments, the isolated polynucleotide encodes an amino acid sequence having at least 85%, at least 86%, at least 87%, at least 88%, at least 89%, at least 90%, at least 910%, at least 92%, at least 93%, at least 94%, at least 95%, at least 96%, at least 97%, at least 98%, at least 99%, or 100% sequence identity to amino acids 203-736 of SEQ ID NO: 12.

In certain embodiments, the isolated polynucleotide encodes amino acids 1-736 of SEQ ID NO: 12. In certain embodiments, the isolated polynucleotide encodes amino acids 137-736 of SEQ ID NO: 12. In certain embodiments, the isolated polynucleotide encodes amino acids 203-736 of SEQ ID NO: 12. In certain embodiments, the isolated polynucleotide encodes SEQ ID NO: 12.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 14. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-736 of SEQ ID NO: 14. In certain embodiments, the isolated polynucleotide encodes an amino acid sequence having at least 85%, at least 86%, at least 87%, at least 88%, at least 89%, at least 90%, at least 910%, at least 92%, at least 93%, at least 94%, at least 95%, at least 96%, at least 97%, at least 98%, at least 99%, or 100% sequence identity to amino acids 204-736 of SEQ ID NO: 14.

In certain embodiments, the isolated polynucleotide encodes amino acids 1-736 of SEQ ID NO: 14. In certain embodiments, the isolated polynucleotide encodes amino acids 138-736 of SEQ ID NO: 14. In certain embodiments, the isolated polynucleotide encodes amino acids 204-736 of SEQ ID NO: 14. In certain embodiments, the isolated polynucleotide encodes SEQ ID NO: 14.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 16. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 16. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 16.

In certain embodiments, the isolated polynucleotide encodes amino acids 1-734 of SEQ ID NO: 16. In certain embodiments, the isolated polynucleotide encodes amino acids 137-734 of SEQ ID NO: 16. In certain embodiments, the isolated polynucleotide encodes amino acids 202-734 of SEQ ID NO: 16. In certain embodiments, the isolated polynucleotide encodes SEQ ID NO: 16.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 18. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-735 of SEQ ID NO: 18. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-735 of SEQ ID NO: 18.

In certain embodiments, the isolated polynucleotide encodes amino acids 1-735 of SEQ ID NO: 18. In certain embodiments, the isolated polynucleotide encodes amino acids 138-735 of SEQ ID NO: 18. In certain embodiments, the isolated polynucleotide encodes amino acids 203-735 of SEQ ID NO: 18. In certain embodiments, the isolated polynucleotide encodes SEQ ID NO: 18.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 20. In certain embodiments, the isolated polynucleotide encodes an amino acid sequence having at least 85%, at least 86%, at least 87%, at least 88%, at least 89%, at least 90%, at least 910%, at least 92%, at least 93%, at least 94%, at least 95%, at least 96%, at least 97%, at least 98%, at least 99%, or 100% sequence identity to amino acids 137-736 of SEQ ID NO: 20. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-736 of SEQ ID NO: 20.

In certain embodiments, the isolated polynucleotide encodes amino acids 1-736 of SEQ ID NO: 20. In certain embodiments, the isolated polynucleotide encodes amino acids 137-736 of SEQ ID NO: 20. In certain embodiments, the isolated polynucleotide encodes amino acids 202-736 of SEQ ID NO: 20. In certain embodiments, the isolated polynucleotide encodes SEQ ID NO: 20.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 22. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-736 of SEQ ID NO: 22. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-736 of SEQ ID NO: 22.

In certain embodiments, the isolated polynucleotide encodes amino acids 1-736 of SEQ ID NO: 22. In certain embodiments, the isolated polynucleotide encodes amino acids 137-736 of SEQ ID NO: 22. In certain embodiments, the isolated polynucleotide encodes amino acids 203-736 of SEQ ID NO: 22. In certain embodiments, the isolated polynucleotide encodes SEQ ID NO: 22.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 24. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-736 of SEQ ID NO: 24. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 204-736 of SEQ ID NO: 24.

In certain embodiments, the isolated polynucleotide encodes amino acids 1-736 of SEQ ID NO: 24. In certain embodiments, the isolated polynucleotide encodes amino acids 138-736 of SEQ ID NO: 24. In certain embodiments, the isolated polynucleotide encodes amino acids 204-736 of SEQ ID NO: 24. In certain embodiments, the isolated polynucleotide encodes SEQ ID NO: 24.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 26. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 26. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 26.

In certain embodiments, the isolated polynucleotide encodes amino acids 1-735 of SEQ ID NO: 26. In certain embodiments, the isolated polynucleotide encodes amino acids 137-735 of SEQ ID NO: 26. In certain embodiments, the isolated polynucleotide encodes amino acids 202-735 of SEQ ID NO: 26. In certain embodiments, the isolated polynucleotide encodes SEQ ID NO: 26.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 28. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 28. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 28.

In certain embodiments, the isolated polynucleotide encodes amino acids 1-734 of SEQ ID NO: 28. In certain embodiments, the isolated polynucleotide encodes amino acids 137-734 of SEQ ID NO: 28. In certain embodiments, the isolated polynucleotide encodes amino acids 202-734 of SEQ ID NO: 28. In certain embodiments, the isolated polynucleotide encodes SEQ ID NO: 28.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 30. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 30. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 30.

In certain embodiments, the isolated polynucleotide encodes amino acids 1-734 of SEQ ID NO: 30. In certain embodiments, the isolated polynucleotide encodes amino acids 137-734 of SEQ ID NO: 30. In certain embodiments, the isolated polynucleotide encodes amino acids 202-734 of SEQ ID NO: 30. In certain embodiments, the isolated polynucleotide encodes SEQ ID NO: 30.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 32. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 32. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 32.

In certain embodiments, the isolated polynucleotide encodes amino acids 1-735 of SEQ ID NO: 32. In certain embodiments, the isolated polynucleotide encodes amino acids 137-735 of SEQ ID NO: 32. In certain embodiments, the isolated polynucleotide encodes amino acids 202-735 of SEQ ID NO: 32. In certain embodiments, the isolated polynucleotide encodes SEQ ID NO: 32.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 34. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 34. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 34.

In certain embodiments, the isolated polynucleotide encodes amino acids 1-735 of SEQ ID NO: 34. In certain embodiments, the isolated polynucleotide encodes amino acids 137-735 of SEQ ID NO: 34. In certain embodiments, the isolated polynucleotide encodes amino acids 202-735 of SEQ ID NO: 34. In certain embodiments, the isolated polynucleotide encodes SEQ ID NO: 34.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 36. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-735 of SEQ ID NO: 36. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-735 of SEQ ID NO: 36.

In certain embodiments, the isolated polynucleotide encodes amino acids 1-735 of SEQ ID NO: 36. In certain embodiments, the isolated polynucleotide encodes amino acids 138-735 of SEQ ID NO: 36. In certain embodiments, the isolated polynucleotide encodes amino acids 203-735 of SEQ ID NO: 36. In certain embodiments, the isolated polynucleotide encodes SEQ ID NO: 36.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-731 of SEQ ID NO: 38. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 134-731 of SEQ ID NO: 38. In certain embodiments, the isolated polynucleotide encodes an amino acid sequence having at least 85%, at least 86%, at least 87%, at least 88%, at least 89%, at least 90%, at least 910%, at least 92%, at least 93%, at least 94%, at least 95%, at least 96%, at least 97%, at least 98%, at least 99%, or 100% sequence identity to amino acids 199-731 of SEQ ID NO: 38.

In certain embodiments, the isolated polynucleotide encodes amino acids 1-731 of SEQ ID NO: 38. In certain embodiments, the isolated polynucleotide encodes amino acids 134-731 of SEQ ID NO: 38. In certain embodiments, the isolated polynucleotide encodes amino acids 199-731 of SEQ ID NO: 38. In certain embodiments, the isolated polynucleotide encodes SEQ ID NO: 38.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 40. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 40. In certain embodiments, the isolated polynucleotide encodes an amino acid sequence having at least 85%, at least 86%, at least 87%, at least 88%, at least 89%, at least 90%, at least 910%, at least 92%, at least 93%, at least 94%, at least 95%, at least 96%, at least 97%, at least 98%, at least 99%, or 100% sequence identity to amino acids 203-735 of SEQ ID NO: 40.

In certain embodiments, the isolated polynucleotide encodes amino acids 1-735 of SEQ ID NO: 40. In certain embodiments, the isolated polynucleotide encodes amino acids 137-735 of SEQ ID NO: 40. In certain embodiments, the isolated polynucleotide encodes amino acids 203-735 of SEQ ID NO: 40. In certain embodiments, the isolated polynucleotide encodes SEQ ID NO: 40.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 42. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-736 of SEQ ID NO: 42. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 204-736 of SEQ ID NO: 42.

In certain embodiments, the isolated polynucleotide encodes amino acids 1-736 of SEQ ID NO: 42. In certain embodiments, the isolated polynucleotide encodes amino acids 138-736 of SEQ ID NO: 42. In certain embodiments, the isolated polynucleotide encodes amino acids 204-736 of SEQ ID NO: 42. In certain embodiments, the isolated polynucleotide encodes SEQ ID NO: 42.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 44. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-736 of SEQ ID NO: 44. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-736 of SEQ ID NO: 44.

In certain embodiments, the isolated polynucleotide encodes amino acids 1-736 of SEQ ID NO: 44. In certain embodiments, the isolated polynucleotide encodes amino acids 137-736 of SEQ ID NO: 44. In certain embodiments, the isolated polynucleotide encodes amino acids 202-736 of SEQ ID NO: 44. In certain embodiments, the isolated polynucleotide encodes SEQ ID NO: 44.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 46. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-736 of SEQ ID NO: 46. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-736 of SEQ ID NO: 46.

In certain embodiments, the isolated polynucleotide encodes amino acids 1-736 of SEQ ID NO: 46. In certain embodiments, the isolated polynucleotide encodes amino acids 137-736 of SEQ ID NO: 46. In certain embodiments, the isolated polynucleotide encodes amino acids 203-736 of SEQ ID NO: 46. In certain embodiments, the isolated polynucleotide encodes SEQ ID NO: 46.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 48. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 48. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 48.

In certain embodiments, the isolated polynucleotide encodes amino acids 1-734 of SEQ ID NO: 48. In certain embodiments, the isolated polynucleotide encodes amino acids 137-734 of SEQ ID NO: 48. In certain embodiments, the isolated polynucleotide encodes amino acids 202-734 of SEQ ID NO: 48. In certain embodiments, the isolated polynucleotide encodes SEQ ID NO: 48.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 50. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 50. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 50.

In certain embodiments, the isolated polynucleotide encodes amino acids 1-734 of SEQ ID NO: 50. In certain embodiments, the isolated polynucleotide encodes amino acids 137-734 of SEQ ID NO: 50. In certain embodiments, the isolated polynucleotide encodes amino acids 202-734 of SEQ ID NO: 50. In certain embodiments, the isolated polynucleotide encodes SEQ ID NO: 50.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 52. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 52. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 52.

In certain embodiments, the isolated polynucleotide encodes amino acids 1-734 of SEQ ID NO: 52. In certain embodiments, the isolated polynucleotide encodes amino acids 137-734 of SEQ ID NO: 52. In certain embodiments, the isolated polynucleotide encodes amino acids 202-734 of SEQ ID NO: 52. In certain embodiments, the isolated polynucleotide encodes SEQ ID NO: 52.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 54. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-735 of SEQ ID NO: 54. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-735 of SEQ ID NO: 54.

In certain embodiments, the isolated polynucleotide encodes amino acids 1-735 of SEQ ID NO: 54. In certain embodiments, the isolated polynucleotide encodes amino acids 138-735 of SEQ ID NO: 54. In certain embodiments, the isolated polynucleotide encodes amino acids 203-735 of SEQ ID NO: 54. In certain embodiments, the isolated polynucleotide encodes SEQ ID NO: 54.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 56. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 56. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 56.

In certain embodiments, the isolated polynucleotide encodes amino acids 1-735 of SEQ ID NO: 56. In certain embodiments, the isolated polynucleotide encodes amino acids 137-735 of SEQ ID NO: 56. In certain embodiments, the isolated polynucleotide encodes amino acids 202-735 of SEQ ID NO: 56. In certain embodiments, the isolated polynucleotide encodes SEQ ID NO: 56.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 58. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 58. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 58.

In certain embodiments, the isolated polynucleotide encodes amino acids 1-735 of SEQ ID NO: 58. In certain embodiments, the isolated polynucleotide encodes amino acids 137-735 of SEQ ID NO: 58. In certain embodiments, the isolated polynucleotide encodes amino acids 202-735 of SEQ ID NO: 58. In certain embodiments, the isolated polynucleotide encodes SEQ ID NO: 58.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 60. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 60. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 60.

In certain embodiments, the isolated polynucleotide encodes amino acids 1-735 of SEQ ID NO: 60. In certain embodiments, the isolated polynucleotide encodes amino acids 137-735 of SEQ ID NO: 60. In certain embodiments, the isolated polynucleotide encodes amino acids 202-735 of SEQ ID NO: 60. In certain embodiments, the isolated polynucleotide encodes SEQ ID NO: 60.

In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 62. In certain embodiments, the isolated polynucleotide encodes an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 62. In certain embodiments, the isolated polynucleotide encodes an amino acid sequence having at least 85%, at least 86%, at least 87%, at least 88%, at least 89%, at least 90%, at least 910%, at least 92%, at least 93%, at least 94%, at least 95%, at least 96%, at least 97%, at least 98%, at least 99%, or 100% sequence identity to amino acids 202-735 of SEQ ID NO: 62.

In certain embodiments, the isolated polynucleotide encodes amino acids 1-735 of SEQ ID NO: 62. In certain embodiments, the isolated polynucleotide encodes amino acids 137-735 of SEQ ID NO: 62. In certain embodiments, the isolated polynucleotide encodes amino acids 202-735 of SEQ ID NO: 62. In certain embodiments, the isolated polynucleotide encodes SEQ ID NO: 62.

In certain embodiments, the present disclosure provides an isolated polynucleotide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the nucleic acid sequence set forth in any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, and 61.

In certain embodiments, the isolated polynucleotide comprises the nucleic acid sequence set forth in SEQ ID NO: 1. In certain embodiments, the isolated polynucleotide comprises the nucleic acid sequence set forth in SEQ ID NO: 3. In certain embodiments, the isolated polynucleotide comprises the nucleic acid sequence set forth in SEQ ID NO: 5. In certain embodiments, the isolated polynucleotide comprises the nucleic acid sequence set forth in SEQ ID NO: 7. In certain embodiments, the isolated polynucleotide comprises the nucleic acid sequence set forth in SEQ ID NO: 9. In certain embodiments, the isolated polynucleotide comprises the nucleic acid sequence set forth in SEQ ID NO: 11. In certain embodiments, the isolated polynucleotide comprises the nucleic acid sequence set forth in SEQ ID NO: 13. In certain embodiments, the isolated polynucleotide comprises the nucleic acid sequence set forth in SEQ ID NO: 15. In certain embodiments, the isolated polynucleotide comprises the nucleic acid sequence set forth in SEQ ID NO: 17. In certain embodiments, the isolated polynucleotide comprises the nucleic acid sequence set forth in SEQ ID NO: 19. In certain embodiments, the isolated polynucleotide comprises the nucleic acid sequence set forth in SEQ ID NO: 21. In certain embodiments, the isolated polynucleotide comprises the nucleic acid sequence set forth in SEQ ID NO: 23. In certain embodiments, the isolated polynucleotide comprises the nucleic acid sequence set forth in SEQ ID NO: 25. In certain embodiments, the isolated polynucleotide comprises the nucleic acid sequence set forth in SEQ ID NO: 27. In certain embodiments, the isolated polynucleotide comprises the nucleic acid sequence set forth in SEQ ID NO: 29. In certain embodiments, the isolated polynucleotide comprises the nucleic acid sequence set forth in SEQ ID NO: 31. In certain embodiments, the isolated polynucleotide comprises the nucleic acid sequence set forth in SEQ ID NO: 33. In certain embodiments, the isolated polynucleotide comprises the nucleic acid sequence set forth in SEQ ID NO: 35. In certain embodiments, the isolated polynucleotide comprises the nucleic acid sequence set forth in SEQ ID NO: 37. In certain embodiments, the isolated polynucleotide comprises the nucleic acid sequence set forth in SEQ ID NO: 39. In certain embodiments, the isolated polynucleotide comprises the nucleic acid sequence set forth in SEQ ID NO: 41. In certain embodiments, the isolated polynucleotide comprises the nucleic acid sequence set forth in SEQ ID NO: 43. In certain embodiments, the isolated polynucleotide comprises the nucleic acid sequence set forth in SEQ ID NO: 45. In certain embodiments, the isolated polynucleotide comprises the nucleic acid sequence set forth in SEQ ID NO: 47. In certain embodiments, the isolated polynucleotide comprises the nucleic acid sequence set forth in SEQ ID NO: 49. In certain embodiments, the isolated polynucleotide comprises the nucleic acid sequence set forth in SEQ ID NO: 51. In certain embodiments, the isolated polynucleotide comprises the nucleic acid sequence set forth in SEQ ID NO: 53. In certain embodiments, the isolated polynucleotide comprises the nucleic acid sequence set forth in SEQ ID NO: 55. In certain embodiments, the isolated polynucleotide comprises the nucleic acid sequence set forth in SEQ ID NO: 57. In certain embodiments, the isolated polynucleotide comprises the nucleic acid sequence set forth in SEQ ID NO: 59. In certain embodiments, the isolated polynucleotide comprises the nucleic acid sequence set forth in SEQ ID NO: 61.

In certain embodiments, the isolated polynucleotide consists of the nucleic acid sequence set forth in SEQ ID NO: 1. In certain embodiments, the isolated polynucleotide consists of the nucleic acid sequence set forth in SEQ ID NO: 3. In certain embodiments, the isolated polynucleotide consists of the nucleic acid sequence set forth in SEQ ID NO: 5. In certain embodiments, the isolated polynucleotide consists of the nucleic acid sequence set forth in SEQ ID NO: 7. In certain embodiments, the isolated polynucleotide consists of the nucleic acid sequence set forth in SEQ ID NO: 9. In certain embodiments, the isolated polynucleotide consists of the nucleic acid sequence set forth in SEQ ID NO: 11. In certain embodiments, the isolated polynucleotide consists of the nucleic acid sequence set forth in SEQ ID NO: 13. In certain embodiments, the isolated polynucleotide consists of the nucleic acid sequence set forth in SEQ ID NO: 15. In certain embodiments, the isolated polynucleotide consists of the nucleic acid sequence set forth in SEQ ID NO: 17. In certain embodiments, the isolated polynucleotide consists of the nucleic acid sequence set forth in SEQ ID NO: 19. In certain embodiments, the isolated polynucleotide consists of the nucleic acid sequence set forth in SEQ ID NO: 21. In certain embodiments, the isolated polynucleotide consists of the nucleic acid sequence set forth in SEQ ID NO: 23. In certain embodiments, the isolated polynucleotide consists of the nucleic acid sequence set forth in SEQ ID NO: 25. In certain embodiments, the isolated polynucleotide consists of the nucleic acid sequence set forth in SEQ ID NO: 27. In certain embodiments, the isolated polynucleotide consists of the nucleic acid sequence set forth in SEQ ID NO: 29. In certain embodiments, the isolated polynucleotide consists of the nucleic acid sequence set forth in SEQ ID NO: 31. In certain embodiments, the isolated polynucleotide consists of the nucleic acid sequence set forth in SEQ ID NO: 33. In certain embodiments, the isolated polynucleotide consists of the nucleic acid sequence set forth in SEQ ID NO: 35. In certain embodiments, the isolated polynucleotide consists of the nucleic acid sequence set forth in SEQ ID NO: 37. In certain embodiments, the isolated polynucleotide consists of the nucleic acid sequence set forth in SEQ ID NO: 39. In certain embodiments, the isolated polynucleotide consists of the nucleic acid sequence set forth in SEQ ID NO: 41. In certain embodiments, the isolated polynucleotide consists of the nucleic acid sequence set forth in SEQ ID NO: 43. In certain embodiments, the isolated polynucleotide consists of the nucleic acid sequence set forth in SEQ ID NO: 45. In certain embodiments, the isolated polynucleotide consists of the nucleic acid sequence set forth in SEQ ID NO: 47. In certain embodiments, the isolated polynucleotide consists of the nucleic acid sequence set forth in SEQ ID NO: 49. In certain embodiments, the isolated polynucleotide consists of the nucleic acid sequence set forth in SEQ ID NO: 51. In certain embodiments, the isolated polynucleotide consists of the nucleic acid sequence set forth in SEQ ID NO: 53. In certain embodiments, the isolated polynucleotide consists of the nucleic acid sequence set forth in SEQ ID NO: 55. In certain embodiments, the isolated polynucleotide consists of the nucleic acid sequence set forth in SEQ ID NO: 57. In certain embodiments, the isolated polynucleotide consists of the nucleic acid sequence set forth in SEQ ID NO: 59. In certain embodiments, the isolated polynucleotide consists of the nucleic acid sequence set forth in SEQ ID NO: 61.

In certain embodiments, the polynucleotide is optimized, e.g., by codon/RNA optimization, replacement with heterologous signal sequences, and/or elimination of mRNA instability elements. Methods to generate optimized polynucleotides for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In certain embodiments, it can be desirable to alter one or more codons to encode a conservative mutation, e.g., a similar amino acid with similar chemical structure and properties and/or function as the original amino acid. Such methods can increase expression of the encoded capsid protein relative to the expression of the capsid encoded by polynucleotides that have not been optimized.

In another aspect the present disclosure provides a vector comprising a polynucleotide disclosed herein. Suitable vectors, include, without limitation, plasmids, viruses, cosmids, artificial chromosomes, linear DNA, and mRNA. In certain embodiments, the vector is a plasmid or a viral vector. In certain embodiments, the vector is a retrovirus vector, a herpes virus vector, a baculovirus vector, or an adenovirus vector. In certain embodiments, the vector is an expression vector.

Vectors (e.g., expression vectors) can be introduced into cells (using any techniques known in the art) for propagation of the vector and/or for expression of an AAV capsid protein encoded by the vector. Accordingly, in another aspect, the instant disclosure provides a recombinant cell comprising a polynucleotide or a vector (e.g., an expression vector) disclosed herein. And further, in another aspect, the instant disclosure provides a method of producing an AAV capsid protein, the method comprising culturing the recombinant cell under conditions whereby the polynucleotide is expressed and the capsid is produced.

Suitable vectors, include, without limitation, plasmids, minimal vectors (e.g., minicircles, Nanoplasmids™, doggybones, MIDGE vectors, and the like), viruses, cosmids, artificial chromosomes, linear DNA, and mRNA. In certain embodiments, the first nucleic acid vector and/or the second nucleic acid vector is a DNA plasmid or a DNA minimal vector. Any DNA plasmid or DNA minimal vector that can accommodate the necessary vector elements can be used for the first nucleic acid vector and the second nucleic acid vector. Suitable DNA minimal vectors include, without limitation, linear covalently closed DNA (e.g., ministring DNA), linear covalently closed dumbbell shaped DNA (e.g., doggybone DNA, dumbbell DNA), minicircles, Nanoplasmids™, minimalistic immunologically defined gene expression (MIDGE) vectors, and others known to those of skill in the art. DNA minimal vectors and their methods of production are described in, e.g., U.S. Patent Application Nos. 20100233814, 20120282283, 20130216562, 20150218565, 20150218586, 20160008488, 20160215296, 20160355827, 20190185924, 20200277624, and 20210010021, all of which are herein incorporated by reference in their entireties.

A variety of host cells and expression vector systems can be utilized to express the capsid proteins described herein. Such expression systems represent vehicles by which the coding sequences of interest can be produced and subsequently purified, but also represent cells which can, when transformed or transfected with the appropriate nucleotide coding sequences, express a capsid protein described herein in situ. These include but are not limited to microorganisms such as bacteria (e.g., E. coli and B. subtilis) transformed with, e.g., recombinant bacteriophage DNA, plasmid DNA or cosmid DNA expression vectors containing capsid protein coding sequences; yeast (e.g., Saccharomyces Pichia) transformed with, e.g., recombinant yeast expression vectors containing capsid protein coding sequences; insect cell systems infected with, e.g., recombinant virus expression vectors (e.g., baculovirus) containing capsid protein coding sequences; plant cell systems (e.g., green algae such as Chlamydomonas reinhardlii) infected with, e.g., recombinant virus expression vectors (e.g., cauliflower mosaic virus, CaMV; tobacco mosaic virus, TMV) or transformed with, e.g., recombinant plasmid expression vectors (e.g., Ti plasmid) containing capsid protein coding sequences; or mammalian cell systems (e.g., COS (e.g., COS1 or COS), CHO, BHK, MDCK, HEK 293, NSO. PER.C6, VERO, CRL7030, HsS78Bst, HeLa, and NIH 3T3, HEK-293T, HepG2, SP210, R1.1, B-W, L-M, BSC1, BSC40, YB/20 and BMT10 cells) harboring, e.g., recombinant expression constructs containing promoters derived from the genome of mammalian cells (e.g., metallothionein promoter) or from mammalian viruses (e.g., the adenovirus late promoter; the vaccinia virus 7.5K promoter). In certain embodiments, cells for expressing the capsid proteins described herein are human cells, e.g., human cell lines. In certain embodiments, a mammalian expression vector is pOptiVEC™ or pcDNA3.3. In certain embodiments, bacterial cells such as Escherichia coli, or eukaryotic cells (e.g., mammalian cells), are used for the expression of a capsid protein. For example, mammalian cells such as CHO or HEK293 cells, in conjunction with a vector such as the major intermediate early gene promoter element from human cytomegalovirus is an effective expression system for capsid proteins disclosed herein.

In bacterial systems, a number of expression vectors can be advantageously selected depending upon the use intended for the capsid protein being expressed. For example, when a large quantity of a capsid protein is to be produced, vectors which direct the expression of high levels of fusion protein products that are readily purified can be desirable. Such vectors include, but are not limited to, the E. coli expression vector pUR278 (Ruether U & Mueller-Hill B (1983) EMBO J 2: 1791-1794), in which the capsid protein coding sequence can be ligated individually into the vector in frame with the lac Z coding region so that a fusion protein is produced; pIN vectors (Inouye S & Inouye M (1985) Nuc Acids Res 13: 3101-3109; Van Heeke G & Schuster S M (1989) J Biol Chem 24: 5503-5509); and the like, all of which are herein incorporated by reference in their entireties. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system. Autographa californica nuclear polyhedrosis virus (AcNPV), for example, can be used as a vector to express foreign genes. The virus grows in Spodoptera frugiperda cells. The capsid protein coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the capsid protein coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the capsid protein molecule in infected hosts (see, e.g., Logan J & Shenk T (1984) PNAS 81(12): 3655-9, which is herein incorporated by reference in its entirety). Specific initiation signals can also be required for efficient translation of inserted capsid protein coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al. (1987) Methods Enzymol. 153: 516-544, which is herein incorporated by reference in its entirety).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NSO (a murine myeloma cell line that does not endogenously produce any immunoglobulin chains). CRL7030, COS (e.g., COS1 or COS), PER.C6, VERO. HsS78Bst, HEK-293T, HepG2, SP210, R1.1, B-W, L-M, BSC1, BSC40, YB/20, BMT10 and HsS78Bst cells.

For long-term, high-yield production of recombinant proteins, stable expression cells can be generated. For example, cell lines which stably express a capsid protein described herein can be engineered.

In certain embodiments, rather than using expression vectors which contain viral origins of replication, host cells can be transformed with a polynucleotide (e.g., DNA or RNA) controlled by appropriate transcriptional regulatory elements (e.g., promoter, enhancer, sequences, transcription terminators, polyadenylation sites, etc.), and a selectable marker. Following the introduction of polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express a capsid protein described herein or a fragment thereof.

A number of selection systems can be used, including but not limited to the herpes simplex virus thymidine kinase (Wigler M et al. (1977) Cell 11(1): 223-32); hypoxanthineguanine phosphoribosyltransferase (Szvbalska E H & Szvbalski W (1962) PNAS 48(12): 2026-2034); and adenine phosphoribosyltransferase (Lowy I et al. (1980) Cell 22(3): 817-23) genes in tk-, hgprt- or aprt-cells, respectively, all of which are herein incorporated by reference in their entireties. Also, antimetabolite resistance can be used as the basis of selection for the following genes: dhfr, which confers resistance to methotrexate (Wigler M et al. (1980) PNAS 77(6): 3567-70: O'Hare K et al. (1981) PNAS 78: 1527-31): gpt, which confers resistance to mycophenolic acid (Mulligan R C & Berg P (1981) PNAS 78(4): 2072-6); neo, which confers resistance to the aminoglycoside G-418 (Wu G Y & Wu C H (1991) Biotherapy 3: 87-95; Tolstoshev P (1993) Ann Rev Pharmacol Toxicol 32: 573-596; Mulligan R C (1993) Science 260: 926-932; and Morgan R A & Anderson W F (1993) Ann Rev Biochem 62: 191-217: Nabel G J & Felgner P L (1993) Trends Biotechnol 11(5): 211-5); and hygro, which confers resistance to hygromycin (Santerre R F et al. (1984) Gene 30(1-3): 147-56), all of which are herein incorporated by reference in their entireties. Methods commonly known in the art of recombinant DNA technology can be routinely applied to select the desired recombinant clone and such methods are described, for example, in Ausubel F M et al. (eds.), Current Protocols in Molecular Biology. John Wiley & Sons, NY (1993); Kriegler M, Gene Transfer and Expression, A Laboratory Manual, Stockton Press, NY (1990); and in Chapters 12 and 13, Dracopoli N C et al. (eds.), Current Protocols in Human Genetics, John Wiley & Sons, NY (1994): Colbere-Garapin F et al. (1981) J Mol Biol 150: 1-14, all of which are herein incorporated by reference in their entireties.

### IV. AAV COMPOSITIONS

In another aspect, the present disclosure provides an rAAV composition comprising a capsid comprising a recombinant AAV capsid protein disclosed herein.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 2. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 2. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 2.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 4. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-736 of SEQ ID NO: 4. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 204-736 of SEQ ID NO: 4.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 6. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 6. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 6.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to amino acids 1-731 of SEQ ID NO: 8. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 134-731 of SEQ ID NO: 8. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 199-731 of SEQ ID NO: 8.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 10. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-736 of SEQ ID NO: 10. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-736 of SEQ ID NO: 10.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 12. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-736 of SEQ ID NO: 12. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-736 of SEQ ID NO: 12.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 14. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-736 of SEQ ID NO: 14. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 204-736 of SEQ ID NO: 14.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 16. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 16. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 16.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 18. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-735 of SEQ ID NO: 18. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-735 of SEQ ID NO: 18.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 20. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-736 of SEQ ID NO: 20. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-736 of SEQ ID NO: 20.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 22. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-736 of SEQ ID NO: 22. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-736 of SEQ ID NO: 22.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 24. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-736 of SEQ ID NO: 24. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 204-736 of SEQ ID NO: 24.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 26. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 26. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 26.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 28. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 28. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 28.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 30. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 30. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 30.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 32. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 32. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 32.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 34. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 34. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 34.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 36. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-735 of SEQ ID NO: 36. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-735 of SEQ ID NO: 36.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-731 of SEQ ID NO: 38. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 134-731 of SEQ ID NO: 38. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 199-731 of SEQ ID NO: 38.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 40. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 40. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-735 of SEQ ID NO: 40.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 42. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-736 of SEQ ID NO: 42. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 204-736 of SEQ ID NO: 42.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 44. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-736 of SEQ ID NO: 44. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-736 of SEQ ID NO: 44.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-736 of SEQ ID NO: 46. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-736 of SEQ ID NO: 46. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-736 of SEQ ID NO: 46.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 48. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 48. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 48.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 50. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 50. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 50.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-734 of SEQ ID NO: 52. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-734 of SEQ ID NO: 52. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-734 of SEQ ID NO: 52.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 54. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 138-735 of SEQ ID NO: 54. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 203-735 of SEQ ID NO: 54.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 56. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 56. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 56.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 58. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 58. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 58.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 60. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 60. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 60.

In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 1-735 of SEQ ID NO: 62. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 137-735 of SEQ ID NO: 62. In certain embodiments, the rAAV composition comprises a capsid comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 202-735 of SEQ ID NO: 62.

In another aspect, the present disclosure provides an rAAV comprising a capsid comprising one or more recombinant capsid proteins disclosed herein; and an rAAV genome. The rAAVs disclosed herein generally comprise a recombinant genome (e.g., an rAAV genome) packaged within the capsid. The rAAV genome can be of any type that is capable of being packages within an AAV capsid disclosed herein. For example, in certain embodiments, the rAAV genome is a single-stranded DNA genome. In certain embodiments, the rAAV genome is a self-complementary genome, for example as described in U.S. Pat. No. 7,790,154, which is hereby incorporated by reference in its entirety.

In certain embodiments, the rAAV genome comprises a transgene. In certain embodiments, the transgene encodes a polypeptide. In certain embodiments, the transgene encodes a therapeutic protein. In certain embodiments, the transgene encodes an antibody or a fragment thereof (e.g., a Fab, scFv, or full-length antibody). In certain embodiments, the transgene encodes an scFv, nanobody, or VHH. In certain embodiments, the transgene encodes a non-coding RNA. In certain embodiments the transgene comprises one or more sequences encoding an RNA molecule. Suitable RNA molecules include, without limitation, miRNA, shRNA, siRNA, antisense RNA, gRNA, antagomirs, miRNA sponges, RNA aptazymes, RNA aptamers, mRNA, lncRNAs, ribozymes, and synthetic RNAs known in the art.

In certain embodiments, the transgene encodes one or more polypeptides, or a fragment thereof. Such transgenes can comprise the complete coding sequence of a polypeptide, or only a fragment of a coding sequence of a polypeptide. In certain embodiments, the transgene encodes a polypeptide that is useful to treat a disease or disorder in a subject. Suitable polypeptides include, without limitation, β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF, acidic FGF, and FGF21), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β) and soluble fragments thereof, and the like; soluble receptors, such as soluble TNF-a receptors, soluble interleukin receptors (e.g., soluble IL-1 receptors and soluble type II IL-1 receptors), soluble γ/Δ T cell receptors, ligand-binding fragments of a soluble receptor, and the like: enzymes, such as α-glucosidase, imiglucerase, β-glucocerebrosidase, and alglucerase; enzyme activators, such as tissue plasminogen activator; chemokines, such as IP-10, monokine induced by interferon-gamma (Mig), Groa/IL-8, RANTES, MIP-1a, MIP-1β, MCP-1, PF-4, and the like; angiogenic agents, such as vascular endothelial growth factors (VEGFs, e.g., VEGF121, VEGF165, VEGF-C, VEGF-2), glioma-derived growth factor, angiogenin, angiogenin-2; and the like: anti-angiogenic agents, such as a soluble VEGF receptor; protein vaccine; neuroactive peptides, such as nerve growth factor (NGF), bradykinin, cholecystokinin, gastrin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide: relaxin; glial fibrillary acidic protein; follicle stimulating hormone (FSH); human alpha-I antitrypsin; leukemia inhibitory factor (LIF); tissue factors; macrophage activating factors; tumor necrosis factor (TNF); neutrophil chemotactic factor (NCF); tissue inhibitors of metalloproteinases; vasoactive intestinal peptide; angiogenin; angiotrofin; fibrin; hirudin; IL-1 receptor antagonists; ciliary neurotrophic factor (CNTF); brain-derived neurotrophic factor (BDNF); neurotrophins 3 and 4/5 (NT-3 and -4/5): glial cell derived neurotrophic factor (GDNF); aromatic amino acid decarboxylase (AADC): Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase, glucose transporter, aldolase A, β-enolase, glycogen synthase; lysosomal enzymes, such as iduronate-2-sulfatase (12S), and arylsulfatase A; and mitochondrial proteins, such as frataxin.

In certain embodiments, the transgene encodes a protein that may be defective in one or more lysosomal storage diseases. Suitable proteins include, without limitation, α-sialidase, cathepsin A, α-mannosidase, β-mannosidase, glycosylasparaginase, α-fucosidase, α-N-acetylglucosaminidase, β-galactosidase, β-hexosaminidase α-subunit, β-hexosaminidase β-subunit, GM2 activator protein, glucocerebrosidase, Saposin C, Arylsulfatase A, Saposin B, formvl-glycine generating enzyme, β-galactosylceramidase, α-galactosidase A, iduronate sulfatase, α-iduronidase, heparan N-sulfatase, acetyl-CoA transferase, N-acetyl glucosaminidase, β-glucuronidase, N-acetyl glucosamine 6-sulfatase, N-acetylgalactosamine 4-sulfatase, galactose 6-sulfatase, hyaluronidase, α-glucosidase, acid sphingomyelinase, acid ceramidase, acid lipase, capthepsin K, tripeptidyl peptidase, palmitoyl-protein thioesterase, cystinosin, sialin, UDP-N-acetylglucosamine, phosphotransferase γ-subunit, mucolipin-1, LAMP-2, NPC1, CLN3, CLN 6, CLN 8, LYST, MYOV, RAB27A, melanophilin, and AP3 β-subunit.

In certain embodiments, the transgene encodes an antibody or a fragment thereof (e.g., a Fab, scFv. or full-length antibody). Suitable antibodies include, without limitation, muromonab-cd3, efalizumab, tositumomab, daclizumab, nebacumab, catumaxomab, edrecolomab, abciximab, rituximab, basiliximab, palivizumab, infliximab, trastuzumab, adalimumab, ibritumomab tiuxetan, omalizumab, cetuximab, bevacizumab, natalizumab, panitumumab, ranibizumab, eculizumab, certolizumab, ustekinumab, canakinumab, golimumab, ofatumumab, tocilizumab, denosumab, belimumab, ipilimumab, brentuximab vedotin, pertuzumab, raxibacumab, obinutuzumab, alemtuzumab, siltuximab, ramucirumab, vedolizumab, blinatumomab, nivolumab, pembrolizumab, idarucizumab, necitumumab, dinutuximab, secukinumab, mepolizumab, alirocumab, evolocumab, daratumumab, elotuzumab, ixekizumab, reslizumab, olaratumab, bezlotoxumab, atezolizumab, obiltoxaximab, inotuzumab ozogamicin, brodalumab, guselkumab, dupilumab, sarilumab, avelumab, ocrelizumab, emicizumab, benralizumab, gemtuzumab ozogamicin, durvalumab, burosumab, erenumab, galcanezumab, lanadelumab, mogamulizumab, tildrakizumab, cemiplimab, fremanezumab, ravulizumab, emapalumab, ibalizumab, moxetumomab, caplacizumab, romosozumab, risankizumab, polatuzumab, eptinezumab, leronlimab, sacituzumab, brolucizumab, isatuximab, and teprotumumab.

In certain embodiments, the transgene encodes a nuclease. Suitable nucleases include, without limitation, zinc fingers nucleases (ZFN) (see, e.g., Porteus, and Baltimore (2003) Science 300: 763; Miller et al. (2007) Nat. Biotechnol. 25:778-785; Sander et al. (2011) Nature Methods 8:67-69; and Wood et al. (2011) Science 333:307, each of which is hereby incorporated by reference in its entirety); transcription activator-like effectors nucleases (TALEN) (see, e.g., Wood et al. (2011) Science 333:307; Boch et al. (2009) Science 326:1509-1512; Moscou and Bogdanove (2009) Science 326; 1501; Christian et al. (2010) Genetics 186:757-761; Miller et al. (2011) Nat. Biotechnol. 29:143-148: Zhang et al. (2011) Nat. Biotechnol. 29:149-153; and Reyon et al. (2012) Nat. Biotechnol. 30(5): 460-465, each of which is hereby incorporated by reference in its entirety); homing endonucleases; meganucleases (see, e.g., U.S. Patent Publication No. US 2014/0121115, which is hereby incorporated by reference in its entirety); and RNA-guided nucleases (see, e.g., Makarova et al. (2018) The CRISPR Journal 1(5): 325-336; and Adli (2018) Nat. Communications 9:1911, each of which is hereby incorporated by reference in its entirety).

In certain embodiments, the transgene encodes an RNA-guided nuclease. Suitable RNA-guided nucleases include, without limitation, Class I and Class II clustered regularly interspaced short palindromic repeats (CRISPR)-associated nucleases. Class I is divided into types I, III, and IV, and includes, without limitation, type I (Cas3), type I-A (Cas8a, Cas5), type I-B (Cas8b), type I-C(Cas8c), type 1-D (Cas10d), type I-E (Cse1, Cse2), type I-F (Csy1, Csy2, Csy3), type I-U (GSU0054), type III (Cas10), type III-A (Csm2), type III-B (Cmr5), type III-C(Csx10 or Csx11), type III-D (Csx10), and type IV (Csf1). Class II is divided into types II, V, and VI, and includes, without limitation, type II (Cas9), type II-A (Csn2), type II-B (Cas4), type V (Cpf1, C2c1, C2c3), and type VI (Cas13a, Cas13b, Cas13c). RNA-guided nucleases also include naturally-occurring Class II CRISPR nucleases such as Cas9 (Type II) or Cas12a/Cpf1 (Type V), as well as other nucleases derived or obtained therefrom. Exemplary Cas9 nucleases that may be used in the present invention include, but are not limited to, S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

In certain embodiments, the transgene encodes reporter sequences, which upon expression produce a detectable signal. Such reporter sequences include, without limitation, DNA sequences encoding β-lactamase. β-galactosidase (LacZ), alkaline phosphatase, thymidine kinase, green fluorescent protein (GFP), red fluorescent protein (RFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

In certain embodiments, the rAAV genome comprises a transcriptional regulatory element (TRE) operably linked to the transgene, to control expression of an RNA or polypeptide encoded by the transgene. In certain embodiments, the TRE comprises a constitutive promoter. In certain embodiments, the TRE can be active in any mammalian cell (e.g., any human cell). In certain embodiments, the TRE is active in a broad range of human cells. Such TREs may comprise constitutive promoter and/or enhancer elements including any of those described herein, and any of those known to one of skill in the art. In certain embodiments, the TRE comprises an inducible promoter. In certain embodiments, the TRE may be a tissue-specific TRE, i.e., it is active in specific tissue(s) and/or organ(s). A tissue-specific TRE comprises one or more tissue-specific promoter and/or enhancer elements, and optionally one or more constitutive promoter and/or enhancer elements. A skilled artisan would appreciate that tissue-specific promoter and/or enhancer elements can be isolated from genes specifically expressed in the tissue by methods well known in the art.

Suitable promoters include, e.g., cytomegalovirus promoter (CMV) (Stinski et al. (1985) Journal of Virology 55(2): 431-441): CMV early enhancer/chicken R-actin (CBA) promoter/rabbit β-globin intron (CAG) (Miyazaki et al. (1989) Gene 79(2): 269-277); CB^{SB} (Jacobson et al. (2006) Molecular Therapy 13(6): 1074-1084); human elongation factor 1α promoter (EF1α) (Kim et al. (1990) Gene 91 (2): 217-223); human phosphoglycerate kinase promoter (PGK) (Singer-Sam et al. (1984) Gene 32(3): 409-417); mitochondrial heavy-strand promoter (Lodeiro et al. (2012) PNAS 109(17): 6513-6518); and ubiquitin promoter (Wulff et al. (1990) FEBS Letters 261: 101-105). In certain embodiments, the TRE comprises a cytomegalovirus (CMV) promoter/enhancer, an SV40 promoter, a chicken beta actin (CBA) promoter, an smCBA promoter, a human elongation factor 1 alpha (EF1α) promoter, a minute virus of mouse (MVM) intron which comprises transcription factor binding sites, a human phosphoglycerate kinase (PGK1) promoter, a human ubiquitin C (Ubc) promoter, a human beta actin promoter, a human neuron-specific enolase (ENO2) promoter, a human beta-glucuronidase (GUSB) promoter, a rabbit beta-globin element, a human calmodulin 1 (CALM1) promoter, a human ApoE/C-I hepatic control region (HCR1), a human al-antitrypsin (hAAT) promoter, an extended HCR1, a HS-CRM8 element of an hAAT promoter, a human transthyretin (TTR) promoter, and/or a human Methyl-CpG Binding Protein 2 (MeCP2) promoter. Any of the TREs described herein can be combined in any order to drive efficient transcription. For example, a transfer genome may comprise a TRE comprising a CMV enhancer, a CBA promoter, and the splice acceptor from exon 3 of the rabbit beta-globin gene, collectively called a CAG promoter. For example, a transfer genome may comprise a TRE comprising a hybrid of CMV enhancer and CBA promoter followed by a splice donor and splice acceptor, collectively called a CASI promoter region. For example, a transfer genome may comprise a TRE comprising a HCR1 and hAAT promoter (also referred to as an LP1 promoter).

In certain embodiments, the TRE is liver-specific. Exemplary liver-specific TREs may comprise one or more elements from, without limitation, the ApoA-I promoter, the ApoA-II promoter, the ApoA-IV promoter, the ApoB promoter, the ApoC-I promoter, the ApoC-II promoter, the ApoC-III promoter, the ApoE promoter, the albumin promoter, the α-fetoprotein promoter, the phosphoenolpyruvate carboxykinase 1 (PCK1) promoter, the phosphoenolpyruvate carboxykinase 2 (PCK2) promoter, the transthyretin (TTR) promoter, the α-antitrypsin (AAT or SERPINA1) promoter, the TK (thymidine kinase) promoter, the hemopexin promoter, the alcohol dehydrogenase 6 promoter, the cholesterol 7alpha-hydroxylase promoter, the factor IX promoter, the α-microglobulin promoter, the SV40 promoter, the CMV promoter, the Rous Sarcoma Virus-LTR promoter and the HBV promoter.

In certain embodiments, the TRE is muscle specific. Exemplary muscle-specific TREs may comprise one or more elements from, without limitation, the human skeletal muscle α-actin (HSA) promoter, the muscle creatine kinase (MCK) promoter, the MHCK7 promoter, the dMCK promoter, the tMCK promoter, the CK6 promoter, the CK8 promoter, the CK8e promoter, the human desmin (DES) promoter or variant thereof, the cardiac troponin T (cTnT) promoter, the myosin light-chain (MLC2v) promoter, the human α-myosin heavy chain gene (αMHC) promoter, the MLC promoter, the human troponin I (TNNI1) promoter, the ΔUSEx3 promoter, the SPcΔ5-12 promoter, the SP-301 promoter, the MH promoter, and the Sk-CRM4/DES promoter.

In certain embodiments, the native promoter for the transgene may be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In another aspect, the instant disclosure provides pharmaceutical compositions comprising an AAV as disclosed herein together with a pharmaceutically acceptable excipient, adjuvant, diluent, vehicle or carrier, or a combination thereof. A “pharmaceutically acceptable carrier” includes any material which, when combined with an active ingredient of a composition, allows the ingredient to retain biological activity and without causing disruptive physiological reactions, such as an unintended immune reaction. Pharmaceutically acceptable carriers include water, phosphate buffered saline, emulsions such as oil/water emulsion, and wetting agents. Compositions comprising such carriers are formulated by well-known conventional methods such as those set forth in Remington's Pharmaceutical Sciences, current ed., Mack Publishing Co., Easton Pa. 18042, USA; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al, 7th ed., Lippincott, Williams, & Wilkins; and Handbook of Pharmaceutical Excipients (2000) A. H. Kibbe et al, 3rd ed. Amer. Pharmaceutical Assoc.

### V. METHODS OF USE

In another aspect, the instant disclosure provides methods for transducing a cell. The methods generally comprise contacting the cell with an rAAV disclosed herein under conditions whereby the cell is transduced. The rAAV disclosed herein can be used to transduce cells in vitro, in vivo and ex vivo.

In another aspect, the instant disclosure provides methods for delivering a transgene into a cell. The methods generally comprise comprising contacting the cell with an rAAV disclosed herein under conditions whereby the cell is transduced and the transgene is expressed.

The rAAV disclosed herein can comprise a transgene under the control of a TRE. Accordingly, in certain embodiments, the instant disclosure provides methods for expressing a transgene in a cell, the method generally comprising contacting the cell with such an rAAV under conditions whereby the cell is transduced and the transgene is expressed. The transgene can encode a polypeptide and/or an RNA molecule, as described herein. Accordingly, in certain embodiments, the instant disclosure provides methods for producing a polypeptide and/or an RNA molecule in a cell, the method generally comprising contacting the cell with such an rAAV under conditions whereby the cell is transduced and the polypeptide and/or an RNA molecule is produced.

In certain embodiments, the cell is in a subject and the rAAV is administered to the subject. The rAAV disclosed herein can be administered to a subject by all routes suitable for an rAAV, including, without limitation, intravenously, intraperitoneally, subcutaneously, intramuscularly, intrathecally, or intradermally.

In certain embodiments, the subject is a member of any mammalian or non-mammalian species. Suitable subjects include, without limitation, humans, non-human primates, canines, felines, ungulates (e.g., equine, bovine, swine (e.g., pig)), avians, rodents (e.g., rats, mice), and other subjects. In certain embodiments, the subject is canine. In certain embodiments, the subject is human.

In another aspect, the invention provides an rAAV as disclosed herein for use in medicine. In another aspect, the invention provides an rAAV as disclosed herein for use as therapy. In another aspect, the invention provides an rAAV as disclosed herein for use as a medicament.

### VI. ADENO-ASSOCIATED VIRUS PACKAGING SYSTEMS

In another aspect, the instant disclosure provides packaging systems for recombinant preparation of a recombinant adeno-associated virus (rAAV) disclosed herein. Such packaging systems generally comprise: a first nucleotide encoding one or more AAV Rep proteins; a second nucleotide encoding a capsid protein of any of the AAVs as disclosed herein; and a third nucleotide sequence comprising any of the rAAV genome sequences as disclosed herein, wherein the packaging system is operative in a cell for enclosing the transfer genome in the capsid to form the AAV.

In certain embodiments, the packaging system comprises a first vector comprising the first nucleotide sequence encoding the one or more AAV Rep proteins and the second nucleotide sequence encoding the AAV capsid protein, and a second vector comprising the third nucleotide sequence comprising the rAAV genome. As used in the context of a packaging system as described herein, a “vector” refers to a nucleic acid molecule that is a vehicle for introducing nucleic acids into a cell (e.g., a plasmid, a virus, a cosmid, an artificial chromosome, etc.). In certain embodiments of the packaging system, the packaging system further comprises a fourth nucleotide sequence comprising one or more helper virus genes. In certain embodiments, the fourth nucleotide sequence comprises adenoviral E2, E4, and VA genes. In certain embodiments of the packaging system, the packaging system further comprises a third vector (e.g., a helper virus vector), comprising the fourth nucleotide sequence. The third vector may be an independent third vector, integral with the first vector, or integral with the second vector.

In certain embodiments, the packaging system comprises a first vector comprising the first nucleotide sequence encoding one or more AAV Rep proteins, the second nucleotide sequence encoding one or more recombinant AAV capsid protein as disclosed herein, and the third nucleotide sequence comprising any of the rAAV genome sequences as disclosed herein, wherein the packaging system is operative in a cell for enclosing the transfer genome in the capsid to form the AAV. In certain embodiments of the packaging system, the packaging system further comprises a fourth nucleotide sequence comprising one or more helper virus genes. In certain embodiments, the fourth nucleotide sequence comprises adenoviral E2, E4, and VA genes. In certain embodiments of the packaging system, the packaging system further comprises a second vector (e.g., a helper virus vector), comprising the fourth nucleotide sequence. The second vector may be an independent second vector, integral with the first vector.

Any AAV Rep protein can be employed in the packaging systems disclosed herein. In certain embodiments of the packaging system, the Rep nucleotide sequence encodes an AAV2 Rep protein. Suitable AAV2 Rep proteins may include, without limitation, Rep 78/68 or Rep 68/52.

In certain embodiments of the packaging system, the helper virus is selected from the group consisting of adenovirus, herpes virus (including herpes simplex virus (HSV)), poxvirus (such as vaccinia virus), cytomegalovirus (CMV), and baculovirus. In certain embodiments of the packaging system, where the helper virus is adenovirus, the adenovirus genome comprises one or more adenovirus RNA genes selected from the group consisting of E1, E2, E4 and VA. In certain embodiments of the packaging system, where the helper virus is adenovirus, the adenovirus genome comprises one or more adenovirus RNA genes selected from the group consisting of E2, E4, and VA. In certain embodiments of the packaging system, where the helper virus is HSV, the HSV genome comprises one or more of HSV genes selected from the group consisting of UL5/8/52, ICPO, ICP4, ICP22, and UL30/UL42.

In certain embodiments of the packaging system, the vectors (e.g., first, second, and/or third vectors) are contained within one or more plasmids.

In certain embodiments of the packaging system, the first, second, and/or third vectors are contained within one or more recombinant helper viruses. In certain embodiments, the first vector and the third vector are contained within a recombinant helper virus. In certain embodiments, the second vector and the third vector are contained within a recombinant helper virus.

In a further aspect, the disclosure provides a method for recombinant preparation of an AAV as described herein, wherein the method comprises transfecting or transducing a cell with a packaging system as described herein under conditions operative for enclosing the rAAV genome in the capsid to form the rAAV as described herein. Exemplary methods for recombinant preparation of an rAAV include transient transfection (e.g., with one or more transfection plasmids containing a first, and a second, and optionally a third vector as described herein), viral infection (e.g. with one or more recombinant helper viruses, such as a adenovirus, poxvirus (such as vaccinia virus), herpes virus (including HSV, cytomegalovirus, or baculovirus, containing a first, and a second, and optionally a third vector as described herein)), and stable producer cell line transfection or infection (e.g., with a stable producer cell, such as a mammalian or insect cell, containing a Rep nucleotide sequence encoding one or more AAV Rep proteins and/or a Cap nucleotide sequence encoding one or more capsid proteins as described herein, and with a transfer genome as described herein being delivered in the form of a plasmid or a recombinant helper virus).

Accordingly, the instant disclosure provides a packaging system for preparation of an rAAV, wherein the packaging system comprises: a first nucleotide sequence encoding one or more AAV Rep proteins; a second nucleotide sequence encoding a capsid protein of any one of the AAVs described herein; a third nucleotide sequence comprising an rAAV genome sequence of any one of the AAVs described herein; and optionally a fourth nucleotide sequence comprising one or more helper virus genes (e.g., adenoviral E2, E4, and VA genes).

### VII. EXAMPLES

The following examples are offered by way of illustration, and not by way of limitation.

### Example 1: AAV Capsid Library Generation and Liver and/or Muscle Enrichment

A shuffled capsid library was generated using capsid sequences of AAV1 through AAV14, AAVrh8, and AAVrh10 as inputs. Briefly, nucleic acid molecules encoding each AAV capsid were digested to create fragments of approximately 200-400 base pairs in length, gel extracted, mixed, and assembled via PCR. The assembled shuffled capsids were then digested and ligated into a vector for subsequent virus generation. Virus was produced using standard triple plasmid transfection techniques. Virus was then collected and harvested via precipitation with PEG 8000, and non-functional capsids were removed via ultracentrifugation in an iodixanol gradient, and buffer exchange of the 40% iodixanol fraction. See, e.g., Zolotukhin et al. (1999) Gene Therapy 6(6): 973-985. Nucleic acid encoding functional capsids were then extracted from the virus preparation using PCR, and the process of viral production was repeated with the functional viral capsid nucleic acids.

To screen AAV capsids with liver-specific and/or muscle-specific tropism, two dogs were each injected with 5E12 to 6E13 vg/kg of AAV virus. After two weeks, liver tissue was isolated, and RNA was extracted to ensure that proper infection, double stranded DNA creation and production of capsids had occurred. cDNA was then synthesized from RNA using second strand cDNA synthesis, amplified by PCR, digested, and ligated into a vector to generate a liver-specific capsid library. A complete second round of capsid nucleic acid extraction, AAV production and liver infection was performed to show enrichment of capsid as a small, directed evolution experiment in which infectivity into the liver was the selection pressure for the library. Sequencing performed on the original shuffled library as well as after each round of infection was used to determine the enrichment of individual capsids.

Following sequence analysis, five capsids were shown to be enriched at each stage of viral production and liver infection (Table 1).

TABLE 1
Capsids and Sequences

| Capsid | Nucleic Acid Sequence | Amino Acid Sequence |
|---|---|---|
| CL01 | SEQ ID NO: 1 | SEQ ID NO: 2 |
| CL03 | SEQ ID NO: 61 | SEQ ID NO: 62 |
| CL11 | SEQ ID NO: 3 | SEQ ID NO: 4 |
| CL13 | SEQ ID NO: 5 | SEQ ID NO: 6 |
| CL27 | SEQ ID NO: 53 | SEQ ID NO: 54 |

### Example 2: Protein Expression from Liver-Enriched Capsids In Vivo

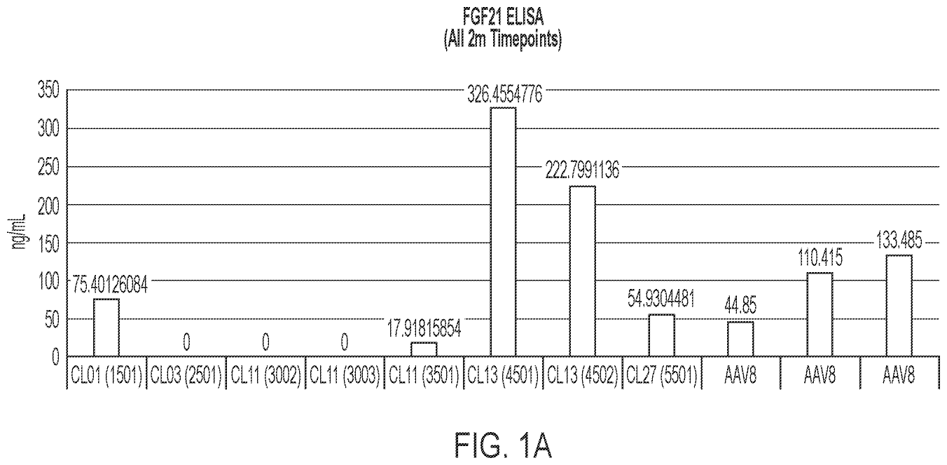

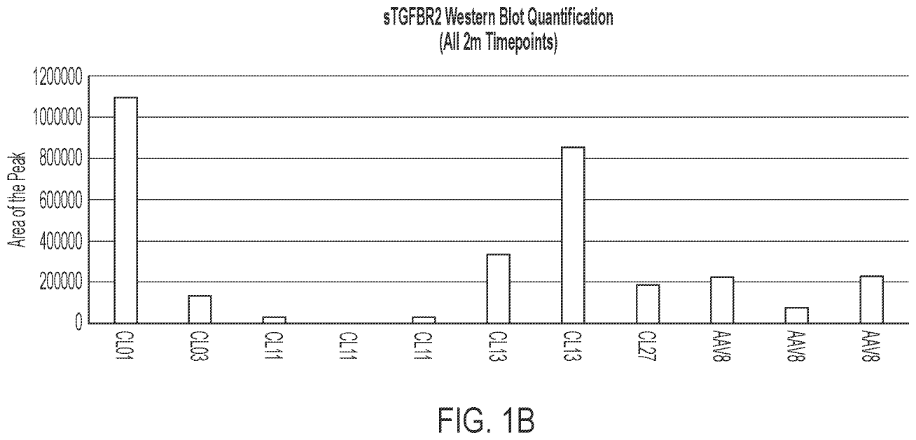

To investigate the properties of the liver-enriched capsids identified in Example 1, an rAAV vector comprising a first coding sequence encoding canine FGF21 and second coding sequence encoding a soluble form of canine transforming growth factor-beta receptor 2 (sTGFbR2; see, e.g., Davidsohn et al. (2019) Proc. Natl. Acad. Sci. USA 116 (47): 23505-23511) was packaged into CL01, CL03, CL11, CL13, CL27, or AAV8 capsids. A total of 30 young adult (1- to 3-year old) naïve male and female Beagle dogs were intravenously administered the CL01, CL03, CL11. CL13, and CL27 packaged viruses at doses of 5×10¹² vg/kg and 1×10¹³ vg/kg (3 dogs at each dose per capsid). Two months post-administration, plasma samples were isolated from blood obtained from the dogs and expression of FGF21 and sTGFbR2 was measured by ELISA (FIG. 1A) or Western blot (FIG. 1B), respectively.

The invention is not to be limited in scope by the specific embodiments described herein. Indeed, various modifications of the invention in addition to those described will become apparent to those skilled in the art from the foregoing description and accompanying FIGURES. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

We claim:

1. A recombinant adeno-associated virus (AAV) capsid protein comprising:
(i) an amino acid sequence having at least 94% sequence identity to amino acids 1-734 of SEQ ID NO: 2, an amino acid sequence having at least 95% sequence identity to amino acids 137-734 of SEQ ID NO: 2, and/or an amino acid sequence having at least 96% sequence identity to amino acids 202-734 of SEQ ID NO: 2;
(ii) an amino acid sequence having at least 96% sequence identity to amino acids 1-736 of SEQ ID NO: 4, an amino acid sequence having at least 95% sequence identity to amino acids 138-736 of SEQ ID NO: 4, and/or an amino acid sequence having at least 96% sequence identity to amino acids 204-736 of SEQ ID NO: 4;
(iii) an amino acid sequence having at least 94% sequence identity to amino acids 1-734 of SEQ ID NO: 6, an amino acid sequence having at least 96% sequence identity to amino acids 137-734 of SEQ ID NO: 6, and/or an amino acid sequence having at least 97% sequence identity to amino acids 202-734 of SEQ ID NO: 6;
(iv) an amino acid sequence having at least 91% sequence identity to amino acids 1-731 of SEQ ID NO: 8, an amino acid sequence having at least 91% sequence identity to amino acids 134-731 of SEQ ID NO: 8, and/or an amino acid sequence having at least 95% sequence identity to amino acids 199-731 of SEQ ID NO: 8;
(v) an amino acid sequence having at least 98% sequence identity to amino acids 1-736 of SEQ ID NO: 10, an amino acid sequence having at least 99% sequence identity to amino acids 138-736 of SEQ ID NO: 10, and/or an amino acid sequence having at least 99% sequence identity to amino acids 203-736 of SEQ ID NO: 10;
(vi) an amino acid sequence having at least 96% sequence identity to amino acids 1-736 of SEQ ID NO: 12, an amino acid sequence having at least 96% sequence identity to amino acids 137-736 of SEQ ID NO: 12, and/or an amino acid sequence having at least 97% sequence identity to amino acids 203-736 of SEQ ID NO: 12;
(vii) an amino acid sequence having at least 96% sequence identity to amino acids 1-736 of SEQ ID NO: 14, an amino acid sequence having at least 95% sequence identity to amino acids 138-736 of SEQ ID NO: 14, and/or an amino acid sequence having at least 96% sequence identity to amino acids 204-736 of SEQ ID NO: 14;
(viii) an amino acid sequence having at least 93% sequence identity to amino acids 1-734 of SEQ ID NO: 16, an amino acid sequence having at least 95% sequence identity to amino acids 137-734 of SEQ ID NO: 16, and/or an amino acid sequence having at least 97% sequence identity to amino acids 202-734 of SEQ ID NO: 16;
(ix) an amino acid sequence having at least 95% sequence identity to amino acids 1-735 of SEQ ID NO: 18, an amino acid sequence having at least 95% sequence identity to amino acids 138-735 of SEQ ID NO: 18, and/or an amino acid sequence having at least 96% sequence identity to amino acids 203-735 of SEQ ID NO: 18;
(x) an amino acid sequence having at least 93% sequence identity to amino acids 1-736 of SEQ ID NO: 20, an amino acid sequence having at least 95% sequence identity to amino acids 137-736 of SEQ ID NO: 20, and/or an amino acid sequence having at least 96% sequence identity to amino acids 202-736 of SEQ ID NO: 20;
(xi) an amino acid sequence having at least 96% sequence identity to amino acids 1-736 of SEQ ID NO: 22, an amino acid sequence having at least 97% sequence identity to amino acids 137-736 of SEQ ID NO: 22, and/or an amino acid sequence having at least 99% sequence identity to amino acids 203-736 of SEQ ID NO: 22;
(xii) an amino acid sequence having at least 97% sequence identity to amino acids 1-736 of SEQ ID NO: 24, an amino acid sequence having at least 96% sequence identity to amino acids 138-736 of SEQ ID NO: 24, and/or an amino acid sequence having at least 97% sequence identity to amino acids 204-736 of SEQ ID NO: 24;
(xiii) an amino acid sequence having at least 99% sequence identity to amino acids 1-735 of SEQ ID NO: 26, an amino acid sequence having at least 99% sequence identity to amino acids 137-735 of SEQ ID NO: 26, and/or an amino acid sequence having at least 99% sequence identity to amino acids 202-735 of SEQ ID NO: 26;
(xiv) an amino acid sequence having at least 92% sequence identity to amino acids 1-734 of SEQ ID NO: 28, an amino acid sequence having at least 94% sequence identity to amino acids 137-734 of SEQ ID NO: 28, and/or an amino acid sequence having at least 96% sequence identity to amino acids 202-734 of SEQ ID NO: 28;
(xv) an amino acid sequence having at least 92% sequence identity to amino acids 1-734 of SEQ ID NO: 30, an amino acid sequence having at least 94% sequence identity to amino acids 137-734 of SEQ ID NO: 30, and/or an amino acid sequence having at least 96% sequence identity to amino acids 202-734 of SEQ ID NO: 30;
(xvi) an amino acid sequence having at least 95% sequence identity to amino acids 1-735 of SEQ ID NO: 32, an amino acid sequence having at least 95% sequence identity to amino acids 137-735 of SEQ ID NO: 32, and/or an amino acid sequence having at least 96% sequence identity to amino acids 202-735 of SEQ ID NO: 32;
(xvii) an amino acid sequence having at least 96% sequence identity to amino acids 1-735 of SEQ ID NO: 34, an amino acid sequence having at least 98% sequence identity to amino acids 137-735 of SEQ ID NO: 34, and/or an amino acid sequence having at least 98% sequence identity to amino acids 202-735 of SEQ ID NO: 34;
(xviii) an amino acid sequence having at least 94% sequence identity to amino acids 1-735 of SEQ ID NO: 36, an amino acid sequence having at least 96% sequence identity to amino acids 138-735 of SEQ ID NO: 36, and/or an amino acid sequence having at least 97% sequence identity to amino acids 203-735 of SEQ ID NO: 36;
(xix) an amino acid sequence having at least 94% sequence identity to amino acids 1-731 of SEQ ID NO: 38, an amino acid sequence having at least 95% sequence identity to amino acids 134-731 of SEQ ID NO: 38, and/or an amino acid sequence having at least 96% sequence identity to amino acids 199-731 of SEQ ID NO: 38;
(xx) an amino acid sequence having at least 94% sequence identity to amino acids 1-735 of SEQ ID NO: 40, an amino acid sequence having at least 97% sequence identity to amino acids 137-735 of SEQ ID NO: 40, and/or an amino acid sequence having at least 97% sequence identity to amino acids 203-735 of SEQ ID NO: 40;
(xxi) an amino acid sequence having at least 96% sequence identity to amino acids 1-736 of SEQ ID NO: 42, an amino acid sequence having at least 96% sequence identity to amino acids 138-736 of SEQ ID NO: 42, and/or an amino acid sequence having at least 96% sequence identity to amino acids 204-736 of SEQ ID NO: 42;
(xxii) an amino acid sequence having at least 94% sequence identity to amino acids 1-736 of SEQ ID NO: 44, an amino acid sequence having at least 96% sequence identity to amino acids 137-736 of SEQ ID NO: 44, and/or an amino acid sequence having at least 97% sequence identity to amino acids 202-736 of SEQ ID NO: 44;
(xxiii) an amino acid sequence having at least 94% sequence identity to amino acids 1-736 of SEQ ID NO: 46, an amino acid sequence having at least 96% sequence identity to amino acids 137-736 of SEQ ID NO: 46, and/or an amino acid sequence having at least 97% sequence identity to amino acids 203-736 of SEQ ID NO: 46;
(xxiv) an amino acid sequence having at least 94% sequence identity to amino acids 1-734 of SEQ ID NO: 48, an amino acid sequence having at least 95% sequence identity to amino acids 137-734 of SEQ ID NO: 48, and/or an amino acid sequence having at least 97% sequence identity to amino acids 202-734 of SEQ ID NO: 48;
(xxv) an amino acid sequence having at least 94% sequence identity to amino acids 1-734 of SEQ ID NO: 50, an amino acid sequence having at least 95% sequence identity to amino acids 137-734 of SEQ ID NO: 50, and/or an amino acid sequence having at least 95% sequence identity to amino acids 202-734 of SEQ ID NO: 50;
(xxvi) an amino acid sequence having at least 93% sequence identity to amino acids 1-734 of SEQ ID NO: 52, an amino acid sequence having at least 94% sequence identity to amino acids 137-734 of SEQ ID NO: 52, and/or an amino acid sequence having at least 96% sequence identity to amino acids 202-734 of SEQ ID NO: 52;
(xxvii) an amino acid sequence having at least 96% sequence identity to amino acids 1-735 of SEQ ID NO: 54, an amino acid sequence having at least 95% sequence identity to amino acids 138-735 of SEQ ID NO: 54, and/or an amino acid sequence having at least 96% sequence identity to amino acids 203-735 of SEQ ID NO: 54;
(xxviii) an amino acid sequence having at least 94% sequence identity to amino acids 1-735 of SEQ ID NO: 56, an amino acid sequence having at least 95% sequence identity to amino acids 137-735 of SEQ ID NO: 56, and/or an amino acid sequence having at least 96% sequence identity to amino acids 202-735 of SEQ ID NO: 56;
(xxix) an amino acid sequence having at least 84% sequence identity to amino acids 1-735 of SEQ ID NO: 58, an amino acid sequence having at least 85% sequence identity to amino acids 137-735 of SEQ ID NO: 58, and/or an amino acid sequence having at least 85% sequence identity to amino acids 202-735 of SEQ ID NO: 58;
(xxx) an amino acid sequence having at least 90% sequence identity to amino acids 1-735 of SEQ ID NO: 60, an amino acid sequence having at least 92% sequence identity to amino acids 137-735 of SEQ ID NO: 60, and/or an amino acid sequence having at least 93% sequence identity to amino acids 202-735 of SEQ ID NO: 60; or
(xxxi) an amino acid sequence having at least 94% sequence identity to amino acids 1-735 of SEQ ID NO: 62, an amino acid sequence having at least 96% sequence identity to amino acids 137-735 of SEQ ID NO: 62, and/or an amino acid sequence having at least 97% sequence identity to amino acids 202-735 of SEQ ID NO: 62.

2. The recombinant AAV capsid protein of claim 1, comprising:
(i) an amino acid sequence having at least 99% sequence identity to amino acids 1-734 of SEQ ID NO: 2, an amino acid sequence having at least 99% sequence identity to amino acids 137-734 of SEQ ID NO: 2, and/or an amino acid sequence having at least 99% sequence identity to amino acids 202-734 of SEQ ID NO: 2;
(ii) an amino acid sequence having at least 99% sequence identity to amino acids 1-736 of SEQ ID NO: 4, an amino acid sequence having at least 99% sequence identity to amino acids 138-736 of SEQ ID NO: 4, and/or an amino acid sequence having at least 99% sequence identity to amino acids 204-736 of SEQ ID NO: 4;
(iii) an amino acid sequence having at least 99% sequence identity to amino acids 1-734 of SEQ ID NO: 6, an amino acid sequence having at least 99% sequence identity to amino acids 137-734 of SEQ ID NO: 6, and/or an amino acid sequence having at least 99% sequence identity to amino acids 202-734 of SEQ ID NO: 6;
(iv) an amino acid sequence having at least 99% sequence identity to amino acids 1-731 of SEQ ID NO: 8, an amino acid sequence having at least 99% sequence identity to amino acids 134-731 of SEQ ID NO: 8, and/or an amino acid sequence having at least 99% sequence identity to amino acids 199-731 of SEQ ID NO: 8;
(v) an amino acid sequence having at least 99% sequence identity to amino acids 1-736 of SEQ ID NO: 10, an amino acid sequence having at least 99% sequence identity to amino acids 138-736 of SEQ ID NO: 10, and/or an amino acid sequence having at least 99% sequence identity to amino acids 203-736 of SEQ ID NO: 10;
(vi) an amino acid sequence having at least 99% sequence identity to amino acids 1-736 of SEQ ID NO: 12, an amino acid sequence having at least 99% sequence identity to amino acids 137-736 of SEQ ID NO: 12, and/or an amino acid sequence having at least 99% sequence identity to amino acids 203-736 of SEQ ID NO: 12;
(vii) an amino acid sequence having at least 99% sequence identity to amino acids 1-736 of SEQ ID NO: 14, an amino acid sequence having at least 99% sequence identity to amino acids 138-736 of SEQ ID NO: 14, and/or an amino acid sequence having at least 99% sequence identity to amino acids 204-736 of SEQ ID NO: 14;
(viii) an amino acid sequence having at least 99% sequence identity to amino acids 1-734 of SEQ ID NO: 16, an amino acid sequence having at least 99% sequence identity to amino acids 137-734 of SEQ ID NO: 16, and/or an amino acid sequence having at least 99% sequence identity to amino acids 202-734 of SEQ ID NO: 16;
(ix) an amino acid sequence having at least 99% sequence identity to amino acids 1-735 of SEQ ID NO: 18, an amino acid sequence having at least 99% sequence identity to amino acids 138-735 of SEQ ID NO: 18, and/or an amino acid sequence having at least 99% sequence identity to amino acids 203-735 of SEQ ID NO: 18;
(x) an amino acid sequence having at least 99% sequence identity to amino acids 1-736 of SEQ ID NO: 20, an amino acid sequence having at least 99% sequence identity to amino acids 137-736 of SEQ ID NO: 20, and/or an amino acid sequence having at least 99% sequence identity to amino acids 202-736 of SEQ ID NO: 20;
(xi) an amino acid sequence having at least 99% sequence identity to amino acids 1-736 of SEQ ID NO: 22, an amino acid sequence having at least 99% sequence identity to amino acids 137-736 of SEQ ID NO: 22, and/or an amino acid sequence having at least 99% sequence identity to amino acids 203-736 of SEQ ID NO: 22;
(xii) an amino acid sequence having at least 99% sequence identity to amino acids 1-736 of SEQ ID NO: 24, an amino acid sequence having at least 99% sequence identity to amino acids 138-736 of SEQ ID NO: 24, and/or an amino acid sequence having at least 99% sequence identity to amino acids 204-736 of SEQ ID NO: 24;
(xiii) an amino acid sequence having at least 99% sequence identity to amino acids 1-735 of SEQ ID NO: 26, an amino acid sequence having at least 99% sequence identity to amino acids 137-735 of SEQ ID NO: 26, and/or an amino acid sequence having at least 99% sequence identity to amino acids 202-735 of SEQ ID NO: 26;
(xiv) an amino acid sequence having at least 99% sequence identity to amino acids 1-734 of SEQ ID NO: 28, an amino acid sequence having at least 99% sequence identity to amino acids 137-734 of SEQ ID NO: 28, and/or an amino acid sequence having at least 99% sequence identity to amino acids 202-734 of SEQ ID NO: 28;
(xv) an amino acid sequence having at least 99% sequence identity to amino acids 1-734 of SEQ ID NO: 30, an amino acid sequence having at least 99% sequence identity to amino acids 137-734 of SEQ ID NO: 30, and/or an amino acid sequence having at least 99% sequence identity to amino acids 202-734 of SEQ ID NO: 30;
(xvi) an amino acid sequence having at least 99% sequence identity to amino acids 1-735 of SEQ ID NO: 32, an amino acid sequence having at least 99% sequence identity to amino acids 137-735 of SEQ ID NO: 32, and/or an amino acid sequence having at least 99% sequence identity to amino acids 202-735 of SEQ ID NO: 32;
(xvii) an amino acid sequence having at least 99% sequence identity to amino acids 1-735 of SEQ ID NO: 34, an amino acid sequence having at least 99% sequence identity to amino acids 137-735 of SEQ ID NO: 34, and/or an amino acid sequence having at least 99% sequence identity to amino acids 202-735 of SEQ ID NO: 34;
(xviii) an amino acid sequence having at least 99% sequence identity to amino acids 1-735 of SEQ ID NO: 36, an amino acid sequence having at least 99% sequence identity to amino acids 138-735 of SEQ ID NO: 36, and/or an amino acid sequence having at least 99% sequence identity to amino acids 203-735 of SEQ ID NO: 36;
(xix) an amino acid sequence having at least 99% sequence identity to amino acids 1-731 of SEQ ID NO: 38, an amino acid sequence having at least 99% sequence identity to amino acids 134-731 of SEQ ID NO: 38, and/or an amino acid sequence having at least 99% sequence identity to amino acids 199-731 of SEQ ID NO: 38;
(xx) an amino acid sequence having at least 99% sequence identity to amino acids 1-735 of SEQ ID NO: 40, an amino acid sequence having at least 99% sequence identity to amino acids 137-735 of SEQ ID NO: 40, and/or an amino acid sequence having at least 99% sequence identity to amino acids 203-735 of SEQ ID NO: 40;
(xxi) an amino acid sequence having at least 99% sequence identity to amino acids 1-736 of SEQ ID NO: 42, an amino acid sequence having at least 99% sequence identity to amino acids 138-736 of SEQ ID NO: 42, and/or an amino acid sequence having at least 99% sequence identity to amino acids 204-736 of SEQ ID NO: 42;
(xxii) an amino acid sequence having at least 99% sequence identity to amino acids 1-736 of SEQ ID NO: 44, an amino acid sequence having at least 99% sequence identity to amino acids 137-736 of SEQ ID NO: 44, and/or an amino acid sequence having at least 99% sequence identity to amino acids 202-736 of SEQ ID NO: 44;
(xxiii) an amino acid sequence having at least 99% sequence identity to amino acids 1-736 of SEQ ID NO: 46, an amino acid sequence having at least 99% sequence identity to amino acids 137-736 of SEQ ID NO: 46, and/or an amino acid sequence having at least 99% sequence identity to amino acids 203-736 of SEQ ID NO: 46;
(xxiv) an amino acid sequence having at least 99% sequence identity to amino acids 1-734 of SEQ ID NO: 48, an amino acid sequence having at least 99% sequence identity to amino acids 137-734 of SEQ ID NO: 48, and/or an amino acid sequence having at least 99% sequence identity to amino acids 202-734 of SEQ ID NO: 48;
(xxv) an amino acid sequence having at least 99% sequence identity to amino acids 1-734 of SEQ ID NO: 50, an amino acid sequence having at least 99% sequence identity to amino acids 137-734 of SEQ ID NO: 50, and/or an amino acid sequence having at least 99% sequence identity to amino acids 202-734 of SEQ ID NO: 50;
(xxvi) an amino acid sequence having at least 99% sequence identity to amino acids 1-734 of SEQ ID NO: 52, an amino acid sequence having at least 99% sequence identity to amino acids 137-734 of SEQ ID NO: 52, and/or an amino acid sequence having at least 99% sequence identity to amino acids 202-734 of SEQ ID NO: 52;
(xxvii) an amino acid sequence having at least 99% sequence identity to amino acids 1-735 of SEQ ID NO: 54, an amino acid sequence having at least 99% sequence identity to amino acids 138-735 of SEQ ID NO: 54, and/or an amino acid sequence having at least 99% sequence identity to amino acids 203-735 of SEQ ID NO: 54;
(xxviii) an amino acid sequence having at least 99% sequence identity to amino acids 1-735 of SEQ ID NO: 56, an amino acid sequence having at least 99% sequence identity to amino acids 137-735 of SEQ ID NO: 56, and/or an amino acid sequence having at least 99% sequence identity to amino acids 202-735 of SEQ ID NO: 56;
(xxix) an amino acid sequence having at least 99% sequence identity to amino acids 1-735 of SEQ ID NO: 58, an amino acid sequence having at least 99% sequence identity to amino acids 137-735 of SEQ ID NO: 58, and/or an amino acid sequence having at least 99% sequence identity to amino acids 202-735 of SEQ ID NO: 58;
(xxx) an amino acid sequence having at least 99% sequence identity to amino acids 1-735 of SEQ ID NO: 60, an amino acid sequence having at least 99% sequence identity to amino acids 137-735 of SEQ ID NO: 60, and/or an amino acid sequence having at least 99% sequence identity to amino acids 202-735 of SEQ ID NO: 60; or
(xxxi) an amino acid sequence having at least 99% sequence identity to amino acids 1-735 of SEQ ID NO: 62, an amino acid sequence having at least 99% sequence identity to amino acids 137-735 of SEQ ID NO: 62, and/or an amino acid sequence having at least 99% sequence identity to amino acids 202-735 of SEQ ID NO: 62.

3. The recombinant AAV capsid protein of claim 1 or 2, comprising amino acids 1-734 of SEQ ID NO: 2, amino acids 137-734 of SEQ ID NO: 2, and/or amino acids 202-734 of SEQ ID NO: 2.

4. The recombinant AAV capsid protein of claim 3, comprising SEQ ID NO: 2.

5. The recombinant AAV capsid protein of claim 1 or 2, comprising amino acids 1-736 of SEQ ID NO: 4, amino acids 138-736 of SEQ ID NO: 4, and/or amino acids 204-736 of SEQ ID NO: 4.

6. The recombinant AAV capsid protein of claim 5, comprising SEQ ID NO: 4.

7. The recombinant AAV capsid protein of claim 1 or 2, comprising amino acids 1-734 of SEQ ID NO: 6, amino acids 137-734 of SEQ ID NO: 6, and/or amino acids 202-734 of SEQ ID NO: 6.

8. The recombinant AAV capsid protein of claim 7, comprising SEQ ID NO: 6.

9. The recombinant AAV capsid protein of claim 1 or 2, comprising amino acids 1-731 of SEQ ID NO: 8, amino acids 134-731 of SEQ ID NO: 8, and/or amino acids 199-731 of SEQ ID NO: 8.

10. The recombinant AAV capsid protein of claim 9, comprising SEQ ID NO: 8.

11. The recombinant AAV capsid protein of claim 1 or 2, comprising amino acids 1-736 of SEQ ID NO: 10, amino acids 138-736 of SEQ ID NO: 10, and/or amino acids 203-736 of SEQ ID NO: 10.

12. The recombinant AAV capsid protein of claim 11, comprising SEQ ID NO: 10.

13. The recombinant AAV capsid protein of claim 1 or 2, comprising amino acids 1-736 of SEQ ID NO: 12, amino acids 137-736 of SEQ ID NO: 12, and/or amino acids 203-736 of SEQ ID NO: 12.

14. The recombinant AAV capsid protein of claim 13, comprising SEQ ID NO: 12.

15. The recombinant AAV capsid protein of claim 1 or 2, comprising amino acids 1-736 of SEQ ID NO: 14, amino acids 138-736 of SEQ ID NO: 14, and/or amino acids 204-736 of SEQ ID NO: 14.

16. The recombinant AAV capsid protein of claim 15, comprising SEQ ID NO: 14.

17. The recombinant AAV capsid protein of claim 1 or 2, comprising amino acids 1-734 of SEQ ID NO: 16, amino acids 137-734 of SEQ ID NO: 16, and/or amino acids 202-734 of SEQ ID NO: 16.

18. The recombinant AAV capsid protein of claim 17, comprising SEQ ID NO: 16.

19. The recombinant AAV capsid protein of claim 1 or 2, comprising amino acids 1-735 of SEQ ID NO: 18, amino acids 138-735 of SEQ ID NO: 18, and/or amino acids 203-735 of SEQ ID NO: 18.

20. The recombinant AAV capsid protein of claim 19, comprising SEQ ID NO: 18.

21. The recombinant AAV capsid protein of claim 1 or 2, comprising amino acids 1-736 of SEQ ID NO: 20, amino acids 137-736 of SEQ ID NO: 20, and/or amino acids 202-736 of SEQ ID NO: 20.

22. The recombinant AAV capsid protein of claim 21, comprising SEQ ID NO: 20.

23. The recombinant AAV capsid protein of claim 1 or 2, comprising amino acids 1-736 of SEQ ID NO: 22, amino acids 137-736 of SEQ ID NO: 22, and/or amino acids 203-736 of SEQ ID NO: 22.

24. The recombinant AAV capsid protein of claim 23, comprising SEQ ID NO: 22.

25. The recombinant AAV capsid protein of claim 1 or 2, comprising amino acids 1-736 of SEQ ID NO: 24, amino acids 138-736 of SEQ ID NO: 24, and/or amino acids 204-736 of SEQ ID NO: 24.

26. The recombinant AAV capsid protein of claim 25, comprising SEQ ID NO: 24.

27. The recombinant AAV capsid protein of claim 1 or 2, comprising amino acids 1-735 of SEQ ID NO: 26, amino acids 137-735 of SEQ ID NO: 26, and/or amino acids 202-735 of SEQ ID NO: 26.

28. The recombinant AAV capsid protein of claim 27, comprising SEQ ID NO: 26.

29. The recombinant AAV capsid protein of claim 1 or 2, comprising amino acids 1-734 of SEQ ID NO: 28, amino acids 137-734 of SEQ ID NO: 28, and/or amino acids 202-734 of SEQ ID NO: 28.

30. The recombinant AAV capsid protein of claim 29, comprising SEQ ID NO: 28.

31. The recombinant AAV capsid protein of claim 1 or 2, comprising amino acids 1-734 of SEQ ID NO: 30, amino acids 137-734 of SEQ ID NO: 30, and/or amino acids 202-734 of SEQ ID NO: 30.

32. The recombinant AAV capsid protein of claim 31, comprising SEQ ID NO: 30.

33. The recombinant AAV capsid protein of claim 1 or 2, comprising amino acids 1-735 of SEQ ID NO: 32, amino acids 137-735 of SEQ ID NO: 32, and/or amino acids 202-735 of SEQ ID NO: 32.

34. The recombinant AAV capsid protein of claim 33, comprising SEQ ID NO: 32.

35. The recombinant AAV capsid protein of claim 1 or 2, comprising amino acids 1-735 of SEQ ID NO: 34, amino acids 137-735 of SEQ ID NO: 34, and/or amino acids 202-735 of SEQ ID NO: 34.

36. The recombinant AAV capsid protein of claim 35, comprising SEQ ID NO: 34.

37. The recombinant AAV capsid protein of claim 1 or 2, comprising amino acids 1-735 of SEQ ID NO: 36, amino acids 138-735 of SEQ ID NO: 36, and/or amino acids 203-735 of SEQ ID NO: 36.

38. The recombinant AAV capsid protein of claim 37, comprising SEQ ID NO: 36.

39. The recombinant AAV capsid protein of claim 1 or 2, comprising amino acids 1-731 of SEQ ID NO: 38, amino acids 134-731 of SEQ ID NO: 38, and/or amino acids 199-731 of SEQ ID NO: 38.

40. The recombinant AAV capsid protein of claim 39, comprising SEQ ID NO: 38.

41. The recombinant AAV capsid protein of claim 1 or 2, comprising amino acids 1-735 of SEQ ID NO: 40, amino acids 137-735 of SEQ ID NO: 40, and/or amino acids 203-735 of SEQ ID NO: 40.

42. The recombinant AAV capsid protein of claim 41, comprising SEQ ID NO: 40.

43. The recombinant AAV capsid protein of claim 1 or 2, comprising amino acids 1-736 of SEQ ID NO: 42, amino acids 138-736 of SEQ ID NO: 42, and/or amino acids 204-736 of SEQ ID NO: 42.

44. The recombinant AAV capsid protein of claim 43, comprising SEQ ID NO: 42.

45. The recombinant AAV capsid protein of claim 1 or 2, comprising amino acids 1-736 of SEQ ID NO: 44, amino acids 137-736 of SEQ ID NO: 44, and/or amino acids 202-736 of SEQ ID NO: 44.

46. The recombinant AAV capsid protein of claim 45, comprising SEQ ID NO: 44.

47. The recombinant AAV capsid protein of claim 1 or 2, comprising amino acids 1-736 of SEQ ID NO: 46, amino acids 137-736 of SEQ ID NO: 46, and/or amino acids 203-736 of SEQ ID NO: 46.

48. The recombinant AAV capsid protein of claim 47, comprising SEQ ID NO: 46.

49. The recombinant AAV capsid protein of claim 1 or 2, comprising amino acids 1-734 of SEQ ID NO: 48, amino acids 137-734 of SEQ ID NO: 48, and/or amino acids 202-734 of SEQ ID NO: 48.

50. The recombinant AAV capsid protein of claim 49, comprising SEQ ID NO: 48.

51. The recombinant AAV capsid protein of claim 1 or 2, comprising amino acids 1-734 of SEQ ID NO: 50, amino acids 137-734 of SEQ ID NO: 50, and/or amino acids 202-734 of SEQ ID NO: 50.

52. The recombinant AAV capsid protein of claim 51, comprising SEQ ID NO: 50.

53. The recombinant AAV capsid protein of claim 1 or 2, comprising amino acids 1-734 of SEQ ID NO: 52, amino acids 137-734 of SEQ ID NO: 52, and/or amino acids 202-734 of SEQ ID NO: 52.

54. The recombinant AAV capsid protein of claim 53, comprising SEQ ID NO: 52.

55. The recombinant AAV capsid protein of claim 1 or 2, comprising amino acids 1-735 of SEQ ID NO: 54, amino acids 138-735 of SEQ ID NO: 54, and/or amino acids 203-735 of SEQ ID NO: 54.

56. The recombinant AAV capsid protein of claim 55, comprising SEQ ID NO: 54.

57. The recombinant AAV capsid protein of claim 1 or 2, comprising amino acids 1-735 of SEQ ID NO: 56, amino acids 137-735 of SEQ ID NO: 56, and/or amino acids 202-735 of SEQ ID NO: 56.

58. The recombinant AAV capsid protein of claim 57, comprising SEQ ID NO: 56.

59. The recombinant AAV capsid protein of claim 1 or 2, comprising amino acids 1-735 of SEQ ID NO: 58, amino acids 137-735 of SEQ ID NO: 58, and/or amino acids 202-735 of SEQ ID NO: 58.

60. The recombinant AAV capsid protein of claim 59, comprising SEQ ID NO: 58.

61. The recombinant AAV capsid protein of claim 1 or 2, comprising amino acids 1-735 of SEQ ID NO: 60, amino acids 137-735 of SEQ ID NO: 60, and/or amino acids 202-735 of SEQ ID NO: 60.

62. The recombinant AAV capsid protein of claim 61, comprising SEQ ID NO: 60.

63. The recombinant AAV capsid protein of claim 1 or 2, comprising amino acids 1-735 of SEQ ID NO: 62, amino acids 137-735 of SEQ ID NO: 62, and/or amino acids 202-735 of SEQ ID NO: 62.

64. The recombinant AAV capsid protein of claim 63, comprising SEQ ID NO: 62.

65. The recombinant AAV capsid protein of any one of the preceding claims, wherein the recombinant AAV capsid protein is a variant of a clade A, clade B, clade C, clade D, clade E, clade F, clade C, clade H, clade I, AAVgo.1, AAV3, AAV4, AAV10, AAV11, AAV12, rh.32, rh32.33, rh.33, rh.34, BAAV, or AAV5 capsid protein.

66. An isolated polynucleotide encoding the recombinant AAV capsid protein of any one of claims 1-65.

67. An isolated polynucleotide having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the nucleic acid sequence set forth in any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, and 61.

68. The isolated polynucleotide of claim 30, comprising the nucleic acid sequence set forth in any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, and 61.

69. A vector comprising the polynucleotide of any one of claims 66-68.

70. The vector of claim 69, which is a plasmid or a viral vector.

71. The vector of claim 70, wherein the viral vector is a retrovirus vector, a herpes virus vector, a baculovirus vector, or an adenovirus vector.

72. The vector of any one of claims 69-71, which is an expression vector.

73. A recombinant cell comprising the polynucleotide of any one of claims 66-68 or the vector of any one of claims 69-72.

74. A method of producing a recombinant AAV capsid protein, the method comprising culturing the recombinant cell of claim 73 under conditions where the polynucleotide is expressed, and the capsid protein is produced.

75. A recombinant adeno-associated virus (rAAV) comprising:
(a) a capsid comprising one or more of the recombinant AAV capsid proteins of any one of claims 1-65; and
(b) an rAAV genome.

76. The rAAV of claim 75, wherein the rAAV genome comprises a transgene.

77. The rAAV of claim 76, wherein the transgene encodes a polypeptide.

78. The rAAV of claim 76, wherein the transgene encodes a miRNA, shRNA, siRNA, antisense RNA, gRNA, antagomir, miRNA sponge, RNA aptazyme, RNA aptamer, lncRNA, ribozyme or mRNA.

79. The rAAV of any one of claims 75-78, wherein the transgene is operably linked to a transcriptional regulatory element.

80. A method for transducing a cell, the method comprising contacting the cell with the rAAV of any one of claims 75-79 under conditions whereby the cell is transduced.

81. A method for delivering a transgene in a cell, the method comprising contacting the cell with the rAAV of any one of claims 75-79 under conditions whereby the cell is transduced and the transgene is expressed.

82. The method of claim 80 or 81, wherein the cell is in a subject and the rAAV is administered to the subject, optionally wherein the cell is a muscle cell and/or a liver cell.

83. The method of any one of claims 80-82, wherein the rAAV is administered to the subject intravenously, intraperitoneally, subcutaneously, intramuscularly, intrathecally, or intradermally.

84. The method of claim 82 or 83, wherein the subject is selected from the group consisting of a human subject, a canine subject, a feline subject, and an equine subject.

85. The rAAV of any one 75-79, for use in medicine, for use as therapy, or for use as a medicament.

86. A packaging system for preparation of an rAAV, wherein the packaging system comprises:
(a) a first nucleotide sequence encoding one or more AAV Rep proteins;
(b) a second nucleotide sequence encoding the recombinant AAV capsid protein of any one of claims 1-65; and
(c) a third nucleotide sequence comprising an rAAV genome sequence.

87. The packaging system of claim 86, wherein the packaging system comprises a first vector comprising the first nucleotide sequence and the second nucleotide sequence, and a second vector comprising the third nucleotide sequence.

88. The packaging system of claim 87, further comprising a fourth nucleotide sequence comprising one or more helper virus genes, optionally wherein the fourth nucleotide sequence is comprised within a third vector.

89. The packaging system of claim 88, wherein the fourth nucleotide sequence comprises one or more genes from a virus selected from the group consisting of adenovirus, herpesvirus, vaccinia virus, and cytomegalovirus (CMV).

90. The packaging system of any one of claims 87-89, wherein the first vector, second vector, and/or the third vector is a plasmid.

91. A method for recombinant preparation of an rAAV, the method comprising introducing the packaging system of any one of claims 86-90 into a cell under conditions whereby the rAAV is produced.
